(19) United States
(12) Patent Application Publication
(10) Pub. No.: US 2012/0190577 A1
(43) Pub. Date: Jul. 26, 2012

(54) PROCESSES AND METHODS FOR DIAGNOSIS OF ALZHEIMER'S DISEASE

(75) Inventors: Rich Einstein, Gaithersburg, MD (US); Weiyin Zhou, Derwood, MD (US); Pascale Beurdeley, Fonteay Sous Bois (FR)

(73) Assignee: Exonhit SA, Paris (FR)

(21) Appl. No.: 13/387,148

(22) PCT Filed: Jul. 30, 2010
(86) PCT No.: PCT/EP2010/061087

§ 371 Date: Apr. 17, 2012

Related U.S. Application Data
(60) Provisional application No. 61/257,934, filed on Nov. 4, 2009.

(30) Foreign Application Priority Data
Jul. 31, 2009 (EP) 09305721.4

(51) Int. Cl. C40B 30/04; C12Q 1/68; C40B 40/06

(52) U.S. Cl. 506/9; 506/16; 435/6.12

(57) ABSTRACT

The present application relates to methods and compositions that can be used to diagnose Alzheimer's disease in mammals, most notably humans. Ti describes most notably peripheral blood biomarkers Alzheimer's disease and uses said biomarkers in diagnostic methods. It also relates to tools and/or kits that can be used to implement said methods (reagents, probes, primers, antibodies, arrays or chips, cells, etc.), as well as the preparation and use thereof. The invention can further be used to detect the presence or the advance of Alzheimer's disease in mammals, including during the disease's early phase, as well as to predict the effectiveness of an Alzheimer's disease treatment.

The present application relates to methods and compositions of use in detecting Alzheimer's disease in mammals, in particular in humans. It describes most notably serum markers for Alzheimer's disease and the use thereof in diagnostic methods. It also relates to tools and/or kits of use in implementing said methods (reagents, probes, primers, antibodies, chips or arrays, cells, etc.), and the preparation and use thereof. The invention can be used to detect the presence or progression of Alzheimer's disease in mammals, including in the disease's early phase (including at pre-symptomatic or pre-AD stage).

Alzheimer's disease is the principal cause of dementia and the most common neurodegenerative disease. This disease, which develops progressively, is characterised by memory loss and by a degradation of aptitudes for language, orientation and judgement. The nature of the symptoms, often confused with physiological troubles related to old age, their severity and the age at which they appear vary from individual to individual. This contributes to the difficulty in diagnosing the early stages of the disease.

Examination of the brains of patients suffering from this disease reveals a loss of neurones in the hippocampus, an important region for memory, and in the cerebral cortex, which is involved in reasoning, language and memory. Cholinergic neurones are particularly affected by this depletion.

Another major anomaly observed in the brains of Alzheimer's patients is the accumulation of intracellular and extracellular protein aggregates. Intracellular neurofibrillary aggregates of tau protein appear strongly correlated with dementia severity. Senile plaques formed by the intracellular and extracellular aggregation of beta-amyloid peptide are characteristic of regions in which neurons and glial cells are affected.

It is, however, remarkable to note that these regions of aggregation do not correspond to the sites of synapse depletion characteristic of cognitive function decline.

Genetic studies undertaken on familial forms showed that four genes are associated with development of the disease: APP (amyloid precursor protein; precursor of beta-amyloid peptide), presenilins 1 and 2 (PS1 and PS2) and apolipoprotein E (ApoE). Although mutations or polymorphisms in each of these genes can lead to increased production of beta-amyloid peptide, the mechanisms that govern synaptic and neuronal losses remain poorly understood. In this respect, several hypotheses and mechanisms appear to coexist, thus implying various phenomena:

1 Purely cerebral phenomena involving neurones and glial cells:
oxidative stress, which can be induced most notably by beta-amyloid peptide and modulated by cholesterol metabolism;
changes in calcium flow and excitotoxicity.

2 Inflammatory and immune reaction phenomena;

3 Changes in sex hormones;

4 Hypothyroidism and defects in the regulation of insulin signalling.

Consequently, Alzheimer's disease is characterised by a change in various systems integrating homeostasis control, the attack on certain neurones leading both to an inflammatory reaction involving the immune system and to changes in endocrine regulation. In return, the latter have an impact on the activity and viability of other neurones and on immune functions, these cascading reactions underlining the role not only of neurodegeneration but also of hormone regulation and immune response in the advance of Alzheimer's disease.

Currently there is no robust and specific signature of Alzheimer's disease, most notably from a blood sample, for establishing a diagnosis of this pathology, most notably of the various stages of the disease. Providing an effective diagnostic test, in particular of the early stages of the disease, would enable patients to be treated from the onset of the disease and thus to benefit from a more effective and more tailored treatment, in particular by acetylcholinesterase inhibitors such as galantamine, donepezil and rivastigmine, under optimal conditions.

The present invention provides a response to this need. The invention describes in particular the identification of serum markers for Alzheimer's disease, enabling the development of effective and predictive diagnoses of the presence, severity or advance, or of the risk of developing this disease. The invention thus describes the identification of molecular signatures specifically or preferentially expressed in the blood of patients suffering from Alzheimer's disease, resulting most notably from the complex expression of certain genes undergoing alternative splicing. The invention describes most notably the identification of sequences SEQ ID NOS.: 1-1754, which are present in genes or RNA of blood cells from human subjects, and which are characteristic, alone or in combination, of Alzheimer's disease. The invention can thus provide tools and methods for diagnosing, predicting and/or monitoring the progression of Alzheimer's disease, based on measuring, in the blood of subjects, the expression of one or more genes. The presence of deregulation in the expression of such genes is used to establish (or to confirm) the presence of Alzheimer's disease in a subject.

An object of the invention thus resides in a method to detect or to confirm (in vitro or ex vivo) the presence of Alzheimer's disease in a mammal, comprising the determination of the presence, in a biological sample from the mammal, preferably in a sample (derived) of blood, of a change in the expression of one or more genes or RNAs comprising a sequence selected from SEQ ID NOS.: 1-1754, the presence of such a change being indicative of the presence or risk of developing Alzheimer's disease in this mammal.

Another object of the invention relates to a method to evaluate or monitor the response to a treatment for Alzheimer's disease, comprising a step of measuring the expression of one or, preferably, several genes or RNAs comprising a sequence selected from SEQ ID NOS.: 1 to 1754 before and/or during treatment, and a comparison of the expression thus measured with that measured before treatment or at a earlier stage of treatment, a change in said expression being indicative of a response to the treatment.

Another object of the invention relates to an improvement in the methods of treating Alzheimer's disease, the improvement consisting in measuring the expression of one or, preferably, several genes or RNAs comprising a sequence selected from SEQ ID NOS.: 1 to 1754, in a subject, before and/or during treatment. Measuring expression makes it possible to adapt the treatment as a function of the evolution of the pathology. The treatment is typically a treatment with acetylcholinesterase inhibitors, such as galantamine, donepezil and rivastigmine.

Another object of the invention relates to the use of an acetylcholinesterase inhibitor, such as galantamine, donepezil and rivastigmine, to prepare a drug to treat Alzheimer's disease in a patient exhibiting deregulation of the expression of at least one gene as previously defined.

A change in a gene or RNA designates, in the context of the invention, (i) any change in the expression, namely deregulation of expression levels (e.g., of transcription or translation), deregulation of splicing, leading for example to the appearance of particular spliced forms or a change in the (relative) quantity or the ratio between the various splicing forms; as well as (ii) any change in the structure of the protein produced (appearance or disappearance of truncated, extended or mutated forms, etc.).

As will be described in the text below, the present application describes the identification of changes in splicing among certain genes in the blood of patients suffering from Alzheimer's disease. Any molecule or technique used to measure the expression of these genes in the blood can be implemented within the scope of this invention, such as nucleotide primers, nucleotide probes or specific antibodies, which can be in suspension or in immobilised form, as will be described in detail in the text below.

Thus, another object of the present application relates to a product comprising a support on which are immobilised nucleic acids comprising a sequence complementary to and/or specific of one or, preferably, several genes or RNAs such as previously defined. Preferably, the product includes distinct nucleic acids comprising a complementary and/or specific sequence of at least 5, 10, 20, 30, 40, 50, 60, 100, 150, 200 or more genes or RNAs such as previously defined.

Another object of the present application relates to a product comprising a support on which at least one ligand of a polypeptide coded by a gene or RNA such as defined above is immobilised. Preferably, the product comprises at least 5, 10, 20, 30, 40, 50, 60 or more ligands of various polypeptides selected from the polypeptides mentioned above.

Another object of the present application relates to a kit comprising a compartment, or a box, or container comprising at least one nucleic acid, preferably several, comprising a complementary and/or specific sequence of one or more genes or RNAs such as previously defined and/or one ligand, preferably several, of one or more polypeptides such as previously defined. Preferably, the product comprises at least 5, 10, 20, 30, 40, 50, 60, 100, 150, 200 or more sequences of various nucleic acids and/or ligands selected from the nucleic acids and ligands mentioned above. The kit can further include reagents for a hybridisation or immunological reaction, as well as, if need be, controls and/or instructions.

Another object of the invention relates to the use of a product or kit such as defined above for detecting Alzheimer's disease in a mammalian subject, preferably a human subject.

Another object of the invention relates to the use of a product or kit such as defined above for determining the response to a treatment for Alzheimer's disease or for selecting subjects likely to respond well to a treatment.

Another object of the invention resides in an isolated nucleic acid comprising a sequence selected from SEQ ID NOS.: 1 to 1754 or a fragment thereof having at least 15, 16, 17, 18, 19 or 20 consecutive bases, or a sequence complementary to said bases. Preferably, the inventive nucleic acid does not comprise the complete sequence of a natural gene or RNA. Preferably, it comprises no more than 500 bases, preferably no more than 400 or 300 bases. The inventive nucleic acid is typically synthetic, that is to say, is produced by a non-natural technique (recombinant, in vitro, chemical synthesis, etc.). Examples of nucleic acids of this invention are probes or primers having a length of between 10 and 30 bases, typically.

Another object of the invention resides in a polypeptide coded by a nucleic acid such as defined above.

Markers for Alzheimer's disease

The present invention rests on revealing and characterising biological events characteristic of Alzheimer's disease in human patients, more specifically from peripheral blood cells. These events constitute biomarkers that, when detected in a patient, preferably in combination, make it possible to determine, even at an early stage, the presence of one such disease, or the developmental stage of this disease. Moreover, the inventive markers also can be used to measure the response to a treatment, and/or to select drug candidates.

The identified biological events typically relate to changes in the regulation of gene expression. It can be an issue of partial or total inhibition of the expression of genes or RNAs, or certain forms of genes or RNAs; an increase in the expression of genes or of certain forms of genes or RNAs; or the appearance or disappearance of forms of gene splicing, etc.

The invention thus rests on detecting, in a sample, one or more target molecules advantageously selected among:
a) nucleic acids comprising a sequence selected from SEQ ID NOS.: 1 to 1754, or a distinctive fragment thereof having at least 15, preferably at least 16, 17, 18, 19, 20, 25 or 30 consecutive bases,
b) nucleic acids having a sequence complementary to a sequence according to a),
c) functional analogues of nucleic acids according to a) or b), or
d) polypeptides coded by the nucleic acids according to a) to c).

Nucleic acids of sequences SEQ ID NOS.: 1-1754 were identified by the inventors from samples of blood cells from healthy human subjects or from subjects with Alzheimer's disease, by transcriptome analysis techniques. These sequences are characteristic, in combinations, of Alzheimer's disease. These sequences are designated in the present application as “target” sequences. They are presented in the biological sense of the gene. Their sequence is given in the sequence listing, and they are described in table 1.

The term “functional analogue” preferably indicates a polymorphic variant of sequences SEQ ID NOS: 1 to 1754 present among the human population. In most cases, these polymorphisms are represented by specific variations on the base level, even if other polymorphic configurations also exist. These analogues can be identified by any technique known to those persons skilled in the art, most notably in consideration of the sequences provided in the application and the names of the corresponding genes.

In a particular embodiment, the method comprises the determination of the presence (or the absence or a variation of expression level) of at least one nucleic acid according to a) to c).

In a very specific embodiment, the method is used to detect Alzheimer's disease in a human subject and comprises the determination of the presence (or the absence or a variation of expression level) of at least one nucleic acid according to a) to c).

In a particular alternate implementation, the method comprises the combined determination of the presence or absence or (relative) quantity of at least 5, 10, 15, 20, 30, 40, 50, 60, 70 or more of the target molecules such as defined above. “Combined” determination indicates the fact that an expression (or hybridisation) profile (or signature) involving several markers is established. Combined determination is typically performed simultaneously, that is to say, via a comprehensive expression profile. Nevertheless, the combined determination can also be performed by parallel or sequential measurements of several markers, leading to the identification of a profile. Indeed, the invention makes it possible to establish and to determine an expression profile (or a signature) on a set of markers, in order to evaluate the presence or the risk of developing Alzheimer's disease in a mammal. The expression profile is typically performed using a combination of several markers selected from the targets indicated above, for example containing all of these targets.

In a particular embodiment, the inventive method comprises the determination of the presence (or absence or (relative) quantity), in a biological sample from a mammal, of at least 5 distinct target molecules selected from those defined above, preferably at least 10.

In this respect, the present application describes specific subsets (panels) of target molecules selected from those defined above, which are particularly adapted to the detection of the presence of Alzheimer's disease in patients from a sample of whole blood.

Thus, in a specific embodiment, the inventive method comprises the combined determination of the presence or absence or relative quantity, in a biological sample from a mammal, of nucleic acids of all of a panel's targets comprising markers as defined in items a) to d) above, preferably all of the nucleic acids of one of the panels 1 to 16 defined in the present application (see table 2).

Thus, in a particular embodiment, the inventive method comprises the combined determination of the presence (or absence or (relative) quantity), in a biological sample from a mammal, of all of the nucleic acids of one of panels 1 to 14, or a distinctive fragment thereof having at least 15, preferably at least 16, 17, 18, 19, 20, 25 or 30 consecutive bases, or having a sequence complementary thereto and/or functional analogues thereof, and/or polypeptides coded by said nucleic acids. The examples provided in the present application indeed show that these panels of markers predictively detect the presence or the stage of advance of Alzheimer's disease. In a particular embodiment, the method further comprises the detection of one or more other target molecules such as previously defined.

In a specific embodiment, the inventive method comprises the determination of the presence (or absence or (relative) quantity), in a biological sample from a mammal, of nucleic acids comprising respectively the sequences represented in SEQ ID NOS.: 1 to 1754 or a distinctive fragment thereof having at least 15, preferably at least 16, 17, 18, 19, 20, 25 or 30 consecutive bases, or nucleic acids having a sequence complementary thereto.

A particular object of the invention resides in a method to detect the presence of Alzheimer's disease in a mammal, comprising contacting, under conditions that allow hybridisation between complementary sequences, nucleic acids from a blood sample from a mammal and a set of probes, the set of probes comprising at least one probe comprising all or part of each of the following target nucleic acid sequences, or of their complementary strand: SEQ ID NOS.: 34, 230, 341, 454, 664, 811, 951, 1127, 1136 and 1752, to obtain an expression profile, the expression profile being characteristic of the presence of Alzheimer's disease in the mammal. In other words, the set of probes comprises:
at least one probe comprising all or part of SEQ ID NO: 34 or of the complementary strand thereof;
at least one probe comprising all or part of SEQ ID NO: 230 or of the complementary strand thereof;
at least one probe comprising all or part of SEQ ID NO: 341 or of the complementary strand thereof;
at least one probe comprising all or part of SEQ ID NO: 454 or of the complementary strand thereof;
at least one probe comprising all or part of SEQ ID NO: 664 or of the complementary strand thereof;
at least one probe comprising all or part of SEQ ID NO: 811 or of the complementary strand thereof;
at least one probe comprising all or part of SEQ ID NO: 951 or of the complementary strand thereof;
at least one probe comprising all or part of SEQ ID NO: 1127 or of the complementary strand thereof;
at least one probe comprising all or part of SEQ ID NO: 1136 or of the complementary strand thereof; and
at least one probe comprising all or part of SEQ ID NO: 1752 or of the complementary strand thereof.

In a preferred embodiment of the method, the set of probes comprises, in addition to the probes indicated above, at least one probe comprising all or part of each of the following additional target nucleic acid sequences, or of their complementary strand: SEQ ID NOS.: 35, 316, 593, 666, 855, 1330 and 1498.

Thus, a preferred panel of target nucleic acids comprises at least the nucleic acids comprising sequences SEQ ID NOS.: 34, 35, 230, 316, 341, 454, 593, 664, 666, 811, 855, 951, 1127, 1136, 1330, 1498 and 1752, or their complementary sequence. As illustrated in the examples, various panels of markers were revealed and validated clinically by the inventors, allowing a determination of the presence of Alzheimer's disease in a subject. Panels 15 and 16 defined above comprise a set of markers common to all the significant panels 1-14 described and tested clinically in the present application, and thus constitute two subsets that are particularly informative and representative for detecting Alzheimer's disease.

In a particularly preferred manner, the set of probes comprises at least one specific probe for each nucleic acid of one of the panels 1 to 14 defined in table 2, or for a distinctive fragment thereof having at least 15, preferably at least 16, 17, 18, 19, 20, 25 or 30 consecutive bases, and/or for a complementary strand thereto.

In addition, as will be explained in more detail in the text below, advantageous use is made of probe sets comprising, for each nucleic acid target sequence, several specific probes that are partially overlapping or not overlapping, typically from 1 to 3. In addition, the probes are advantageously immobilised on a support. Moreover, the expression profile is generally analysed by computer software.

Another object of the invention resides in a method to detect the presence or the risk of developing Alzheimer's disease in a mammal, the method comprising detection, from a blood sample from a mammal, of a variation in the levels of nucleic acids complementary to a group of probes such as defined previously, variation being characteristic of the presence of Alzheimer's disease in a mammal.

The invention also makes it possible to define additional panels, comprising at least certain markers such as previously defined, which optionally can be combined with other markers. Such panels can be obtained by testing the presence or absence of these markers in patient samples, to define other predictive combinations.

The invention further relates to the use of a set of probes such as defined previously to detect in vitro or ex vivo the presence of Alzheimer's disease in a subject.

The invention further relates to the use of a set of primers comprising at least one primer comprising all or part of one or more nucleic acid sequence targets mentioned previously, for in vitro or ex vivo detection of Alzheimer's disease.

The invention also relates to a method to detect the presence of Alzheimer's disease in a mammal, comprising contacting, under conditions allowing an amplification reaction, nucleic acids from a blood sample from a mammal and a set of primers, the set of primers comprising at least one primer comprising all or part of each of the following nucleic acid sequences, or of the complementary strand thereof: SEQ ID NOS.: 34, 230, 341, 454, 664, 811, 951, 1127, 1136 and 1752, to obtain a profile or amplification product, the profile or amplification product being characteristic of the presence of Alzheimer's disease in a mammal.

Methods for Detecting a Change in Gene Expression

As indicated previously, a change in a gene or RNA indicates in the context of the invention (i) any change in expression, namely deregulation of expression levels (e.g., of transcription or translation), deregulation of splicing, leading for example to the appearance of particular spliced forms or a change in the (relative) quantity of or relationship between various splicing forms, and (ii) any change in the structure of the protein produced (appearance or disappearance of truncated, extended or mutated forms, etc.).

Various techniques for detecting a species of nucleic acid in a sample can be used in the present invention, such as for example northern blot, selective hybridisation, the use of supports covered with oligonucleotide probes, nucleic acid amplification such as for example RT-PCR, quantitative PCR or ligation-PCR, etc. These methods can include the use of a nucleic probe (for example an oligonucleotide) capable of detecting selectively or specifically the nucleic acid targets in the sample. Amplification can be performed according to various methods known to the person skilled in the art, such as PCR, LCR, transcription mediated amplification (TMA), strand displacement amplification (SDA), NASBA, the use of allele specific oligonucleotides (ASO), allele specific amplification. Detection can also be made using, e.g., Southern blot, single-strand conformation analysis (SSCA), in situ hybridisation (e.g., FISH), gel migration, heteroduplex analysis, NextGen sequencing, etc. If necessary, the quantity of nucleic acid detected can be compared with a reference value, for example a median or mean value observed among patients who do not have Alzheimer's disease, or with a value measured in parallel in a control sample. Thus, it is possible to demonstrate variation in expression levels.

According to a preferred embodiment, the method comprises detection of the presence or absence or (relative) quantity of a nucleic acid according to a) to c) by selective hybridisation or selective amplification.

Selective hybridisation is typically performed using nucleic probes, preferably immobilised on a support, such as a solid or semi-solid support having at least one surface, flat or not, for immobilising nucleic probes. Such supports are, for example, a slide, bead, membrane, filter, column, plate, etc. They can be made out of any compatible material, such as in particular glass, silica, plastic, fiber, metal, polymer, etc. The nucleic probes can be any acid nucleic (DNA, RNA, PNA, etc.), preferably single-strand, comprising a specific sequence of a target molecule such as defined in a) to c) above. The probes typically comprise from 5 to 400 bases, preferably from 8 to 200, more preferentially less than 100, and even more preferentially less than 75, 60, 50, 40 or even 30 bases. The probes can be synthetic oligonucleotides, produced on the basis of inventive sequences SEQ ID NO: 1 to 1754 (target sequences), according to standard synthesis techniques. Such oligonucleotides typically comprise from 10 to 50 bases, preferably from 20 to 40, for example approximately 25 bases. In a particularly advantageous embodiment, so as to improve the detected signal, several different oligonucleotides (or probes) defined from the same target sequence are used to detect the same target molecule (transcribed from the amplification of the patient's RNA to produce either cRNA or cDNA) during hybridisation. This may include specific oligonucleotides of various regions of the same target sequence, or centred differently on a given region. Advantageously, use is made of probe sets comprising 1-3 probes, which can be overlapping or not, wholly or partly, and which are specific for the same target molecule. Use can also be made of probe pairs, in which one member is paired perfectly with the target sequence, and the other presents a mismatch, thus making it possible to estimate background signal. Probes can be designed to hybridise with a region of an exon or an intron, or with an exon-exon, exon-intron or intron-intron junction region. Thus, the probes make it possible to reveal and to distinguish various alternative spliced isoforms.

In a preferred embodiment, use is made of probes whose sequence comprises all or part of a nucleic acid sequence selected from SEQ ID NOS.: 1 to 1754 or of a sequence complementary thereto. In a preferred mode, use is made of nucleic acid probes having a length between 15 and 50 bases, more preferentially between 15 and 40 bases, and whose sequence is identical to a fragment of a sequence selected from SEQ ID NO: 1 to 1754 or of a sequence complementary thereto. The probe can also be designed in the opposite orientation.

In a particularly preferred embodiment, use is made of probe sets, that is to say, sets of 1-3 probes each comprising a section, overlapping or not, of the same nucleic acid sequence selected from SEQ ID NOS.: 1 to 1754 or of the complementary strand thereof.

The probes can be synthesised in advance and then deposited on the support, or synthesised directly in situ, on the support, according to methods known to the person skilled in the art. The probes can also be manufactured by genomic or molecular techniques, for example by amplification, recombination, ligation, etc.

The probes thus defined constitute another object of the present application, as well as the use thereof (primarily in vitro) for detecting Alzheimer's disease in a subject.

Hybridisation can be performed under standard conditions, known to and adjustable by the person skilled in the art (see Sambrook, Fritsch, Maniatis (1989) Molecular Cloning, Cold Spring Harbor Laboratory Press). Most notably, hybridisation can be performed under conditions of high, medium or low stringency, according to the level of sensitivity needed, the quantity of material available, etc. For example, suitable hybridisation conditions include a temperature between 55° C. and 63° C. for 2 hours to 18 hours. Other hybridisation conditions, adapted to high density supports, are for example a hybridisation temperature between 45° C. and 55° C. After hybridisation, various washes can be performed to eliminate the non-hybridised molecules, typically in SSC buffers including SDS, such as a buffer comprising 0.1× to 10×SSC and 0.5% to 0.01% SDS. Other wash buffers containing SSPE, MES, NaCl or EDTA can also be used.

In a typical embodiment, the nucleic acids (or arrays or supports) are pre-hybridised in hybridisation buffer (Rapid Hybrid Buffer, Amersham) typically containing 100 μg/ml of salmon sperm DNA at 65° C. for 30 min. The nucleic acids of the sample are then placed in contact with the probes (typically applied to the support or the array) at 65° C. for 2 hours to 18 hours. Preferably, the nucleic acids of the sample are marked beforehand by any known marker (biotin, radioactive, enzymatic, fluorescent, luminescent, etc.). The supports are then washed in 5×SSC, 0.1% SDS buffer at 65° C. for 30 min., then in a 0.2×SSC, 0.1% SDS buffer. The expression profile is analysed according to standard techniques, such as for example by measuring labelling on the support by means of a suitable instrument (for example InstantImager, Packard Instruments). Hybridisation conditions naturally can be adjusted by the person skilled in the art, for example by modifying hybridisation temperature and/or the salt concentration of the buffer as well as by adding auxiliary substances such as formamide or single-strand DNA.

A particular object of the invention thus resides in a method to detect the presence or the risk of developing Alzheimer's disease in a mammal, or to evaluate the response to a treatment against Alzheimer's disease, comprising contacting, under conditions allowing hybridisation between complementary sequences, nucleic acids from a blood sample from a mammal and a set of probes specific of the target molecules identified previously to obtain an expression profile, the expression profile being characteristic of the presence or the risk of developing Alzheimer's disease in a mammal, or of the effectiveness of the treatment.

A particular object of the invention thus resides in a method to detect the presence of Alzheimer's disease in a mammal, comprising contacting, under conditions allowing hybridisation between complementary sequences, nucleic acids from a blood sample from a mammal and a set of probes specific of the following target molecules at least:
a) nucleic acids comprising the sequences of one of the panels 1 to 14 previously defined, or a distinctive fragment thereof having at least 15, preferably at least 16, 17, 18, 19, 20, 25 or 30 consecutive bases, and/or,
b) nucleic acids having a sequence complementary to sequences according to a), and/or,
c) functional analogues of the nucleic acids according to a) or b),
   to obtain an expression profile, the expression profile being characteristic of the presence of Alzheimer's disease in a mammal.

In the specific embodiments, the inventive processes use in addition other target molecules and/or other probes, most notably the target molecule subsets mentioned in the present application.

Thus, another particular object of the invention resides in a method to detect the presence or the risk of developing Alzheimer's disease in a mammal, comprising contacting, under conditions allowing hybridisation between complementary sequences, nucleic acids from a blood sample from a mammal and a set of probes specific of at least two distinct molecules selected from the following targets:
a) nucleic acids comprising the sequences represented in SEQ ID NOS.: 1 to 1754 or a distinctive fragment thereof having at least 15, preferably at least 16, 17, 18, 19, 20, 25 or 30 consecutive bases, and/or,
b) nucleic acids having a sequence complementary to sequences according to a), and/or,
c) functional analogues of the nucleic acids according to a) or b),
   to obtain an expression profile, the expression profile being characteristic of the presence of Alzheimer's disease in a mammal.

Another particular object of the invention resides in a method to detect the presence of Alzheimer's disease in a mammal, comprising contacting, under conditions allowing hybridisation between complementary sequences, the nucleic acids from a blood sample from a mammal and a set of probes, the set of probes comprising at least one probe comprising all or part of each nucleic acid sequence of one of panels 1-16 or of a sequence complementary thereto to obtain an expression profile, the expression profile being characteristic of the presence of Alzheimer's disease in a mammal.

As explained in further detail in the experimental section, a preferred set according to the invention comprises at least the following probes:
from 1 to 3 probes, overlapping or not, each comprising all or part of sequence SEQ ID NO: 34 or of a sequence complementary thereto;
from 1 to 3 probes, overlapping or not, each comprising all or part of sequence SEQ ID NO: 230 or of a sequence complementary thereto;
from 1 to 3 probes, overlapping or not, each comprising all or part of sequence SEQ ID NO: 341 or of a sequence complementary thereto;
from 1 to 3 probes, overlapping or not, each comprising all or part of sequence SEQ ID NO: 454 or of a sequence complementary thereto;
from 1 to 3 probes, overlapping or not, each comprising all or part of sequence SEQ ID NO: 664 or of a sequence complementary thereto;
from 1 to 3 probes, overlapping or not, each comprising all or part of sequence SEQ ID NO: 811 or of a sequence complementary thereto;
from 1 to 3 probes, overlapping or not, each comprising all or part of sequence SEQ ID NO: 951 or of a sequence complementary thereto;
from 1 to 3 probes, overlapping or not, each comprising all or part of sequence SEQ ID NO: 1127 or of a sequence complementary thereto;
from 1 to 3 probes, overlapping or not, each comprising all or part of sequence SEQ ID NO: 1136 or of a sequence complementary thereto; and
from 1 to 3 probes, overlapping or not, each comprising all or part of sequence SEQ ID NO: 1752 or of a sequence complementary thereto.

As indicated previously, the term “part” advantageously indicates a region of 15 to 50 consecutive nucleotides.

It is understood that the set can comprise, in addition to the 10 probes or groups of probes (probe sets) cited, other probes or probe sets, comprising for example a sequence selected from SEQ ID NO: 1 to 1754 and/or among other sequences.

Thus, another preferred set according to the invention comprises, in addition to the probes mentioned above, at least the following additional probes:
from 1 to 3 probes, overlapping or not, each comprising all or part of sequence SEQ ID NO: 35 or of a sequence complementary thereto;
from 1 to 3 probes, overlapping or not, each comprising all or part of sequence SEQ ID NO: 316 or of a sequence complementary thereto;
from 1 to 3 probes, overlapping or not, each comprising all or part of sequence SEQ ID NO: 593 or of a sequence complementary thereto;
from 1 to 3 probes, overlapping or not, each comprising all or part of sequence SEQ ID NO: 666 or of a sequence complementary thereto;
from 1 to 3 probes, overlapping or not, each comprising all or part of sequence SEQ ID NO: 855 or of a sequence complementary thereto;
from 1 to 3 probes, overlapping or not, each comprising all or part of sequence SEQ ID NO: 1330 or of a sequence complementary thereto; and
from 1 to 3 probes, overlapping or not, each comprising all or part of sequence SEQ ID NO: 1498 or of a sequence complementary thereto.

Panels 1-14 can be defined like the set above. The basic probes constituting these sets are given in the examples.

Another object of the invention resides in a method to detect the presence or the risk of developing Alzheimer's disease in a mammal, comprising the detection, from a blood sample from a mammal, of a variation in the levels of nucleic acids complementary to a set of probes, the set of probes comprising at least one probe comprising all or part of each nucleic acid sequence of one of the panels defined previously, a variation being characteristic of the presence or the risk of developing Alzheimer's disease in a mammal.

The expression profile can be compared with one or more basic profiles, most notably a basic profile characteristic of healthy subjects and/or subjects with Alzheimer's disease, the comparison making it possible to determine the probability that the patient tested has Alzheimer's disease. Typically, the comparison is performed by means of computer software known to the person skilled in the art.

Selective amplification is preferably performed using a primer or a pair of primers to amplify all or part of one of the target nucleic acids in the sample, when the target nucleic acid is present. The primer can be specific for a target sequence such as previously defined according to SEQ ID NO: 1 to 1754, or of a region flanking the target sequences in a nucleic acid of the sample. The primer typically comprises a single-strand nucleic acid, with a length advantageously between 5 and 50 bases, preferably between 5 and 30 bases. Such a primer constitutes another object of the present application, as well as the use thereof (primarily in vitro) for detecting Alzheimer's disease in a subject. The primers can be designed to hybridise with a region of an exon or an intron, or with an exon-exon, exon-intron or intron-intron junction region. Thus, the primers reveal and distinguish various forms of gene splicing.

In this respect, another object of the invention resides in the use of a nucleotide primer or a set of nucleotide primers to amplify all or part of one or, preferably, several genes or RNAs comprising a target sequence according to SEQ ID NO: 1 to 1754, to detect the presence of Alzheimer's disease in a mammal, or to evaluate the response to a treatment against Alzheimer's disease in a mammal, particularly in a human being.

Another specific object of the invention resides in a method to detect the presence of Alzheimer's disease in a mammal, comprising contacting, under conditions allowing amplification, nucleic acids from a blood sample from a mammal and a set of primers specific of at least two distinct molecules selected from the following targets:
a) nucleic acids comprising the sequences represented in SEQ ID NOS.: 1 to 1754 or a distinctive fragment thereof having at least 15, preferably at least 16, 17, 18, 19, 20, 25 or 30 consecutive bases, and/or,
b) nucleic acids having a sequence complementary to sequences according to a), and/or,
c) functional analogues of the nucleic acids according to a) or b),
   to obtain an amplification profile, the amplification profile being characteristic of the presence of Alzheimer's disease in a mammal.

Detection of a Change in a Polypeptide

In another embodiment, the method comprises the determination of the presence or the (relative) quantity of a polypeptide coded by a gene such as defined previously. Revealing or assaying a polypeptide in a sample can be performed by any known technique, most notably by means of a specific ligand, for example an antibody or an antibody fragment or derivative. Preferably, the ligand is a specific antibody of the polypeptide, or a fragment of such an antibody (for example Fab, Fab', CDR, etc.), or a derivative of such an antibody (for example a single-chain variable-fragment antibody, scFv). The ligand is typically immobilised on a support, such as a slide, bead, column, plate, etc. The presence or quantity of target polypeptide in the sample can be detected by revealing a complex between the target and the ligand, for example by using a labelled ligand or by using a second labelled indicator ligand, etc. Immunological techniques that can be used and are well known are ELISA and RIA techniques, etc. If necessary, the quantity of polypeptide detected can be compared with a reference value, for example a median or mean value observed among patients who do not have Alzheimer's disease, or with a value measured in parallel in a control sample. Thus, it is possible to reveal variation in expression levels.

Specific antibodies of target polypeptides can be produced by conventional techniques, most notably by immunization of a non-human animal with an immunogen comprising the polypeptide (or an immunogenic fragment thereof), and recovery of the antibodies (polyclonal) or producing cells (to produce monoclonal antibodies). Production techniques for polyclonal or monoclonal antibodies, single-chain variable-fragment antibodies and human or humanised antibodies are described for example in Harlow et al., A Laboratory Manual, CSH Press, 1988; Ward et al., Nature 341 (1989) 544; Bird et al., Science 242 (1988) 423; WO 94/02602; U.S. Pat. No. 5,223,409; U.S. Pat. No. 5,877,293 and WO 93/01288. The immunogen can be produced by synthesis, or by expression, in a suitable host, of a nucleic acid target such as defined previously. Such an antibody, monoclonal or polyclonal, as well as derivatives thereof having the same antigenic specificity, also constitutes an object of the present application, as well as the use thereof to detect Alzheimer's disease.

Changes in protein expression and/or structure can also be detected by means of techniques, known to the person skilled in the art, involving mass spectroscopy, more generally grouped under the name proteome analysis, in order to detect specific signatures in the blood of patients suffering from Alzheimer's disease.

Implementation of the Method

The inventive method is applicable to any biological sample from the tested mammal, most notably any sample that includes nucleic acids or polypeptides. Specific examples include a sample of blood, plasma, platelets, saliva, urine, stool, etc., more generally any tissue, organ or, advantageously, biological fluid that includes nucleic acids or polypeptides.

In one preferred and particularly advantageous embodiment, the sample is a sample of blood derivative, for example a sample of blood, serum or plasma. Indeed, the invention follows from the identification of blood markers for Alzheimer's disease, and thus enables detection of this pathology without tissue biopsy, but from blood samples alone.

The sample can be obtained by any known technique, for example by drawing, by non-invasive techniques, or from sample collections or banks, etc. Further, the sample can be pre-treated to facilitate access to the target molecules, for example by lysis (mechanical, chemical, enzymatic, etc.), purification, centrifugation, separation, etc. The sample can also be labelled to facilitate the determination of the presence of target molecules (biotin, fluorescent, radioactive, luminescent, chemical or enzymatic labelling, etc.). The nucleic acids of the sample can in addition be separated, treated, enriched, purified, reverse transcribed, amplified, fragmented, etc. In a particular embodiment, the nucleic acids of the sample are RNAs, most notably mRNA of the sample. In a very specific embodiment, the nucleic acids are the product of amplification of RNA, most notably of mRNA, or cDNA prepared from RNA, most notably mRNA of the sample.

In a preferred embodiment, the biological sample is a sample of whole blood, i.e., not having undergone a separation step, which optionally can be diluted.

The invention is applicable to any mammal, preferably humans. The inventive method is particularly useful for the detection of Alzheimer's disease, most notably of the presence or the degree of severity or advance of Alzheimer's disease in a human being. Thus, the data provided in the examples show that the invention can detect the presence of Alzheimer's disease with sensitivity higher than 70% and specificity higher than 70%. Indeed, it is known that the rate of false positives in the current diagnosis of healthy patients on the basis of clinical examination is about 15% to 30% and the rate of false positives in the current diagnosis of AD patients is 15%, on the basis of multiple clinical examinations. A definitive diagnosis can only be established post-mortem from an autopsy of the brain.

A particular object of the present application relates to a method to detect the presence or the evolution of Alzheimer's disease in a human subject, comprising the combined determination of the presence (or absence or (relative) quantity), in a biological sample of the human subject, target molecules selected from:
a) nucleic acids comprising a sequence selected from SEQ ID NO.: 1 to 1754 or a distinctive fragment thereof having at least 15, preferably at least 16, 17, 18, 19, 20, 25 or 30 consecutive bases,
b) nucleic acids having a sequence complementary to a sequence according to a), and,
c) the polypeptides coded by the nucleic acids according to a) or b).

Preferably, the method comprises the combined determination of the presence, absence or quantity of 5, 10, 20, 30, 40, 50 or 60 or more target molecules such as defined above.

Another particular object of the present application relates to a method to detect the presence or the progress of Alzheimer's disease in a human subject, comprising contacting a biological sample from the subject that contains nucleic acids with a product comprising a support on which are immobilised nucleic acids comprising a sequence complementary and/or specific of one or, preferably, several target molecules selected from (i) the nucleic acids comprising a sequence selected from SEQ ID NO: 1 to 1754 or a fragment thereof having at least 15, preferably at least 16, 17, 18, 19, 20, 25 or 30 consecutive bases and (ii) nucleic acids having a sequence complementary to a sequence according to (i), to obtain an expression profile, the profile indicating the presence, degree of severity/advance or risk of developing Alzheimer's disease by said human subject. Preferably, the product includes distinct nucleic acids comprising a sequence complementary and/or specific of at least 5, 10, 20, 30, 40, 50, 60 or more distinct genes or RNAs as mentioned above.

Another object of the present application relates to a product comprising a support on which are immobilised nucleic acids comprising a sequence complementary and/or specific for one or, preferably, several target molecules selected from (i) nucleic acids comprising a sequence selected from SEQ ID NO: 1 to 1754 or a fragment thereof having at least 15, preferably at least 16, 17, 18, 19, 20, 25 or 30 consecutive bases and (ii) nucleic acids having a sequence complementary to a sequence according to (i). Preferably, the product includes distinct nucleic acids comprising a sequence complementary and/or specific of at least 5, 10, 20, 30, 40, 50, 60 or more genes or RNAs such as previously defined.

Another object of the present application relates to a product comprising a support on which is immobilised at least one, preferably several, nucleic acids comprising a sequence selected from SEQ ID NO: 1 to 1754, or a functional analogue thereof. Preferably, the product comprises at least 5, 10, 20, 30, 40, 50, 60 or more various nucleic acids selected from the nucleic acids mentioned above.

Another particular object of the invention relates to a product comprising a support on which is immobilised a set of probes, the set containing at least one probe comprising all or part of each of the following nucleic acid sequences, or of the complementary strand thereof: SEQ ID NOS.: 34, 230, 341, 454, 664, 811, 951, 1127, 1136 and 1752.

Another particular object of the invention relates to a product comprising a support on which is immobilised a set of probes, the set containing at least one probe comprising all or part of each of the following nucleic acid sequences, or of the complementary strand thereof: SEQ ID NOS.: 34, 35, 230, 316, 341, 454, 593, 664, 666, 811, 855, 951, 1127, 1136, 1330, 1498 and 1752.

A preferred inventive product comprises a support on which is immobilised at least one set of probes of distinct nucleic acids comprising a complementary and/or specific sequence of nucleic acids from the one of the panels 1 to 14 defined in table 1.

Typically, the probes are selected from SEQ ID: 1755-6532.

The invention further relates to a kit comprising a compartment or container comprising at least 5, 10, 20, 30, 40, 50, 60 or more various nucleic acids selected from the nucleic acids defined previously.

The invention also relates to a kit comprising a product such as defined previously and reagents for a hybridisation reaction.

The invention further relates to the use of a product or kit defined above for in vitro or ex vivo detection of the presence of Alzheimer's disease, or of the response to a treatment for Alzheimer's disease in a subject.

Another object of the present application relates to a product comprising a support on which is immobilised at least one ligand of a polypeptide coded by a nucleic acid target such as defined above, i.e., a nucleic acid comprising a sequence selected from SEQ ID NO: 1 to 1754, a distinctive fragment thereof having at least 15, preferably at least 16, 17, 18, 19, 20, 25 or 30 consecutive bases, a nucleic acid having a sequence complementary to same or a functional analogue thereof. Preferably, the product comprises at least 5, 10, 20, 30, 40, 50, 60 or more ligands of various polypeptides selected from polypeptides mentioned above.

The support can be any solid or semi-solid support having at least one surface, flat or not (i.e., in two or three dimensions), allowing the immobilisation of nucleic acids or polypeptides. Such supports are for example a slide, bead, membrane, filter, column, plate, etc. They can be made of any compatible material, such as most notably glass, silica, plastic, fibre, metal, polymer, polystyrene, Teflon, etc. The reagents can be immobilised on the surface of the support by known techniques, or, in the case of nucleic acids, synthesised directly in situ on the support. Immobilisation techniques include passive adsorption (Inouye et al., J. Clin. Microbiol. 28 (1990) 1469) and covalent bonding. Techniques are described for example in WO 90/03382 and WO 99/46403. The reagents immobilised on the support can be placed in a predetermined order, to facilitate detection and identification of the complexes formed, and according to a variable and adaptable density.

In one embodiment, the inventive product comprises a multiplicity of synthetic oligonucleotides, of length between 5 and 100 bases, and specific for one or more genes or RNAs such as previously defined.

The products of the invention typically comprise control molecules, which are used to calibrate and/or standardise the results.

Another object of the present application relates to a product comprising a support on which are immobilised nucleic acids comprising all or part of the sequences selected from SEQ ID NO: 1755-6532. These sequences represent specific probes of SEQ ID NO: 1 to 1754. Such a product will be able to advantageously incorporate nucleic acids chosen for their character of not discriminating the population of patients suffering from Alzheimer's disease, such nucleic acids being used as standardisation controls for the product. These nucleic acids can correspond to all or part of the sequences selected from SEQ ID NO: 1755-6532.

Another object of the present application relates to a kit comprising a compartment or container comprising at least one, preferably several, nucleic acids comprising a complementary and/or specific sequence of one or more genes or RNAs such as previously defined and/or one, preferably several ligands of one or more polypeptides such as previously defined. Preferably, the product comprises at least 5, 10, 20, 30, 40, 50, 60 or more various nucleic acids and/or ligands selected from the nucleic acids and ligands mentioned above. In a particular embodiment, the product comprises each of the nucleic acids of sequence SEQ ID NO: 1 to 1754 or a ligand for each of the polypeptide targets such as defined above. In another particular embodiment, the product comprises each of the nucleic acids of sequence SEQ ID NO: 1755-6532. The kit can further comprise reagents for a hybridisation or immunological reaction, as well as, if need be, controls and/or instructions.

Another object of the invention relates to the use of a product or kit such as defined above for the detection of Alzheimer's disease in a mammalian subject, preferably a human subject.

Another object of the invention relates to a nucleic acid of a sequence selected from SEQ ID NO: 1 to 1754, or a distinctive fragment thereof comprising at least 15 consecutive bases, preferably at least 16, 17, 18, 19, 20, 25 or 30 consecutive bases, or a nucleic acid having a sequence complementary to same, or a functional analogue thereof. The invention also relates to a cloning or expression vector comprising said nucleic acids, as well as any recombinant cell comprising one such vector or nucleic acid.

Another object of the invention relates to the use of a nucleic acid comprising a sequence selected from SEQ ID NO: 1 to 1754, or a distinctive fragment thereof comprising at least 15 consecutive bases, preferably at least 16, 17, 18, 19, 20, 25 or 30 consecutive bases, or a nucleic acid having a sequence complementary to same, or a functional analogue thereof, for the detection (primarily in vitro) of Alzheimer's disease in a mammalian subject.

According to a particular example of an embodiment of the invention, a blood sample is drawn from a mammal to be tested. The blood sample is optionally treated in such a way as to make the nucleic acids more accessible, and said nucleic acids are labelled. The nucleic acids are then applied to a product such as defined previously and the expression profile is determined, making it possible to diagnose the presence or absence of Alzheimer's disease in the subject. The inventive method is simple, performed ex vivo, and allows the early detection of Alzheimer's disease from a blood sample.

It is understood that any equivalent technique can be used within the scope of the present application to determine the presence of a target molecule.

Other aspects and advantages of the present invention will appear upon consideration of the following examples, which must be regarded as illustrative and non-restrictive.

### LEGEND TO THE FIGURE

FIG. 1. Configuration of the probes present on the GWSA microarray. A) The exon probes of type B, F and T and the junction probes of type C, D and E optimally cover the splicing events. B) Probes able to cover any single exon jump are also present with the same configuration. Similarly, exon-exon (X), exon-intron (Z) and intron-exon (Y) junction probes are also present.

### LEGEND TO THE TABLES

Table 1. Description of sequences SEQ ID NO: 1 to 6532.

Table 2. List of probe sets of each panel 1 to 14.

### Example 1

### Identification of Biomarkers for Alzheimer's Disease

1.1. Characteristics of the Biological Samples

The examples presented below were initially performed with 177 or 100 among the 177 blood samples (2.5 ml of whole blood, taken in two PaxGene tubes). These samples cover 90 patients diagnosed with probable Alzheimer's disease according to DSM-IV criteria. These patients had a mean age of 78.08 years (standard deviation: 6.67 years). These patients presented a MMSE (Mini-Mental State Examination) score below 27 (average score of 17.16; standard deviation of 6.04). In addition 87 subjects, of comparable age to the AD patients, declared not demented after a clinical examination were also recruited (mean age 69.71 years, standard deviation 6.53 years; mean MMSE of 29.31 with a standard deviation of 0.97).

1.2. Extraction of Total RNA from the Blood Sample

The blood samples were collected directly in PAXGene™ Blood RNA tubes (PreAnalytix, Hombrechtikon, CH). After the step of drawing the blood sample and in order to obtain complete cell lysis, the tubes were left at room temperature for 4 hours and then stored at −20° C. or −80° C. until extraction of the biological material. More precisely, in this protocol, total RNA was extracted using PAXGene Blood RNA® kits (PreAnalytix) by following the manufacturer's recommendations. Briefly, the tubes were centrifuged (10 min, 3,000 g) in order to obtain a pellet of nucleic acids. This pellet was washed and taken up in a buffer containing proteinase K necessary for protein digestion (10 min at 55° C.). An additional centrifugation (3 min, 14,000 g) was performed to eliminate cellular debris and ethanol was added in order to optimise conditions for binding nucleic acids. Total RNA was specifically bound on PAXgene RNA spin columns and, before they were eluted, contaminating DNA was digested using an RNAse-free DNAse set (Qiagen, Hilden, Germany). The quality and quantity of total RNA extracted were evaluated by performing electrophoregrams using an Agilent 2100 Bioanalyser with an RNA 6000 NanoChip kit (Agilent Technologies, Santa Clara, Calif.). Only samples meeting quality standards were used for further analyses.

1.3. Genome-Wide SpliceArray™ (GWSA)

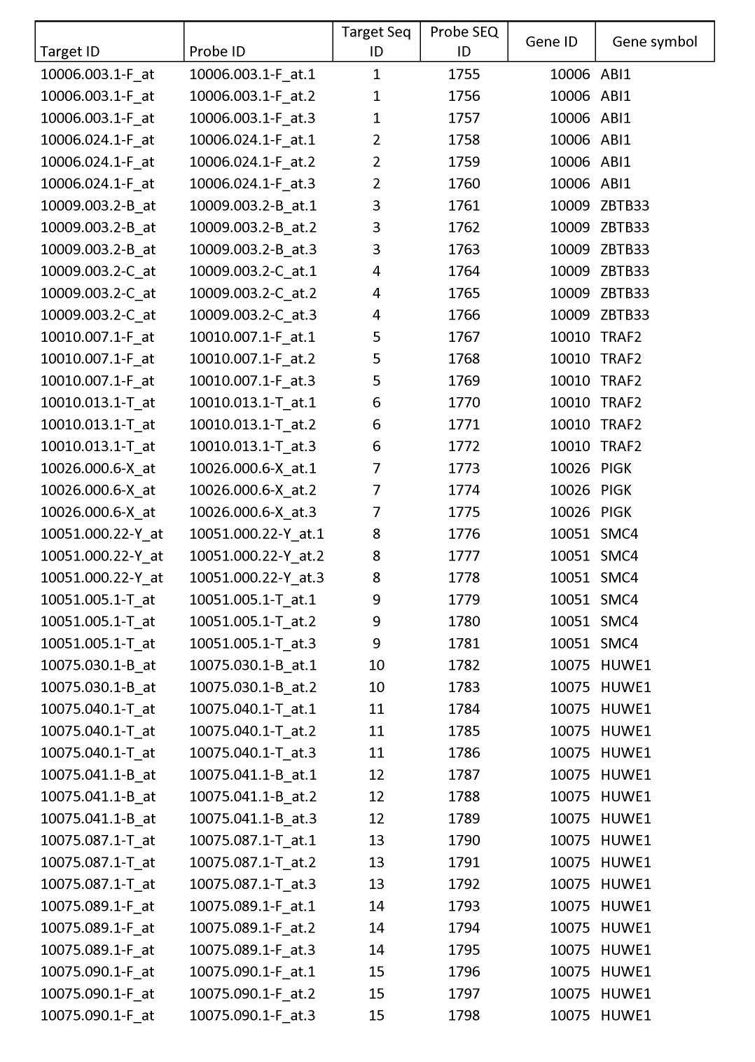

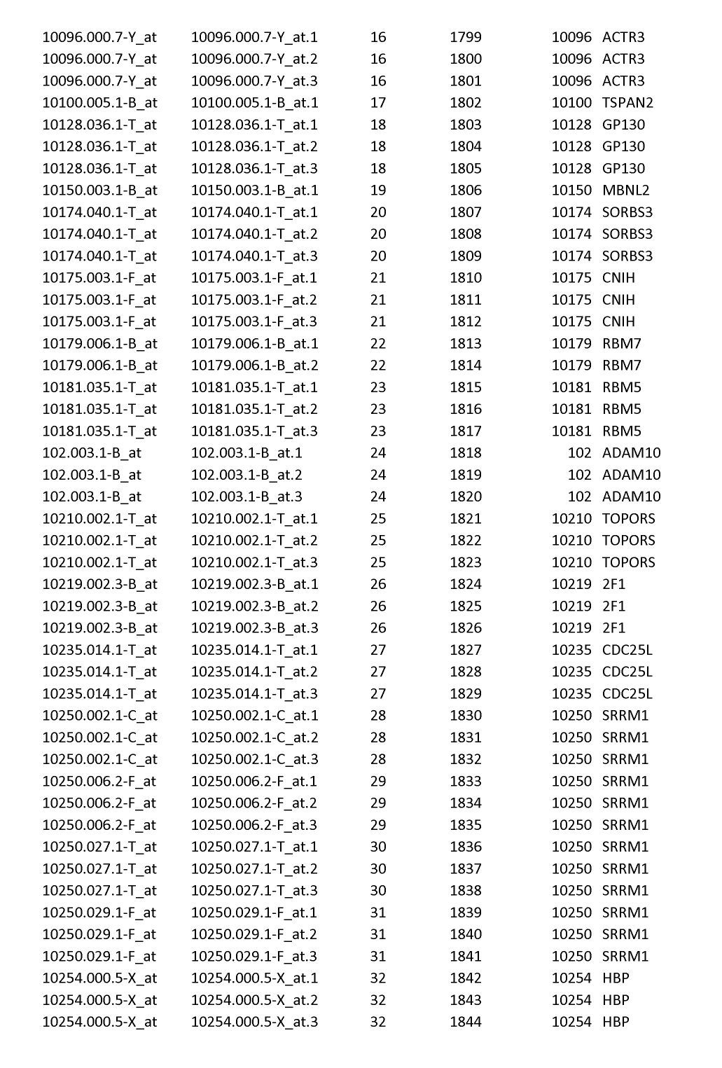

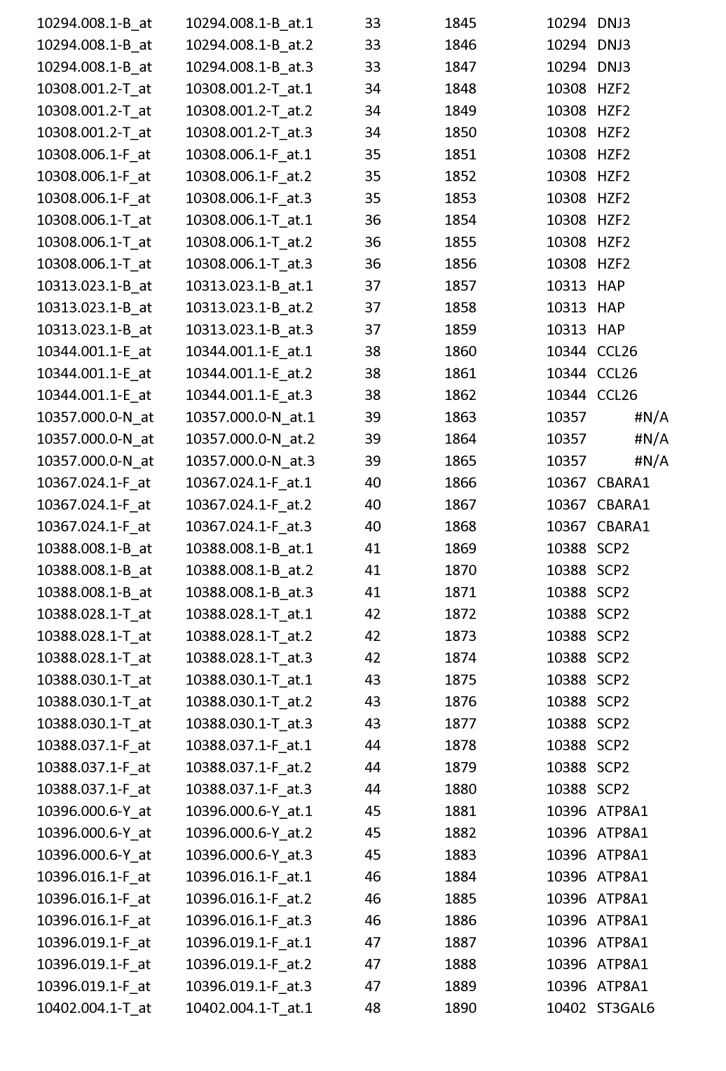

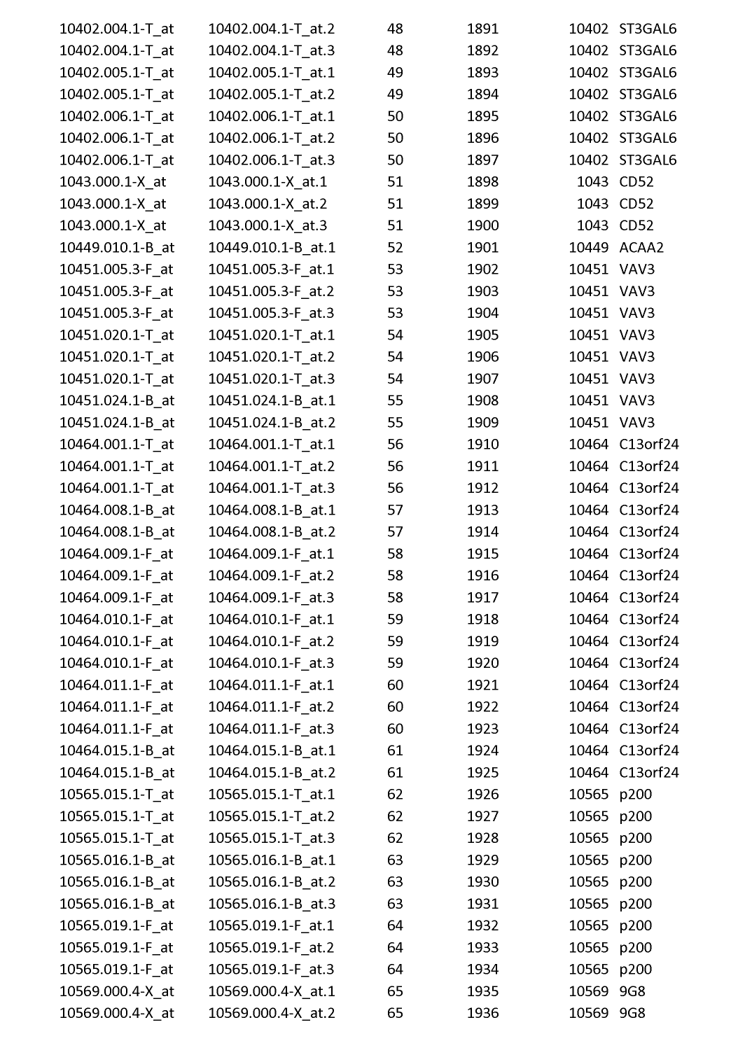

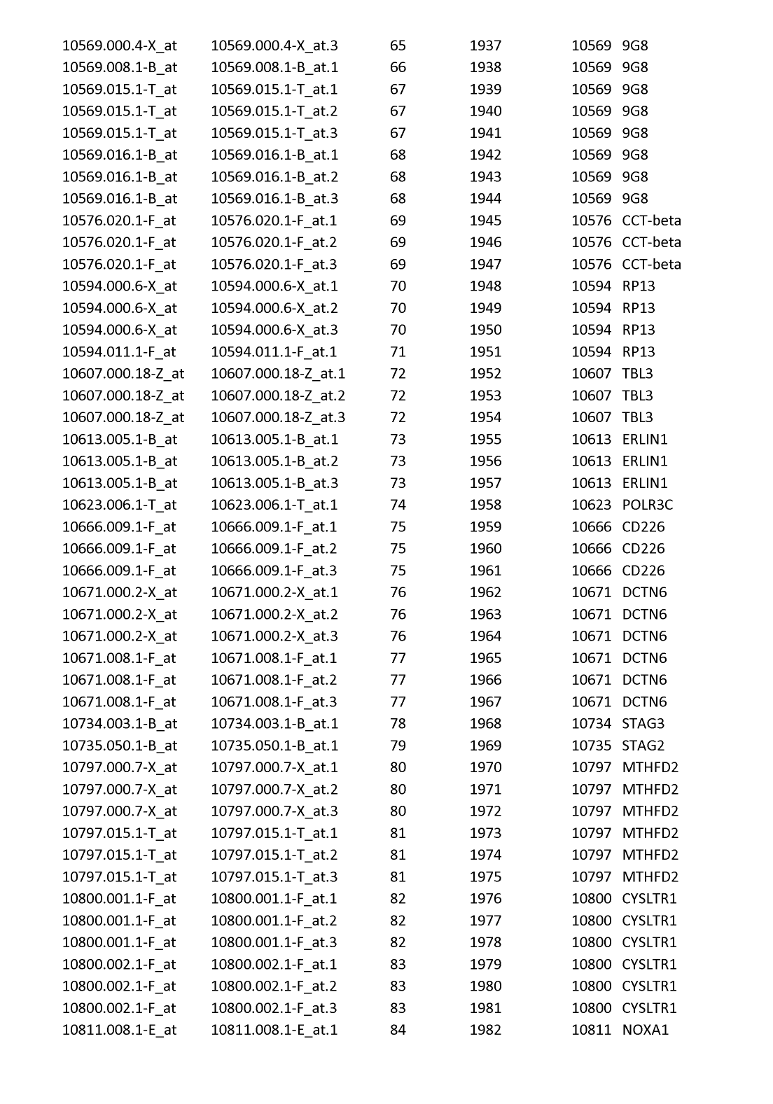

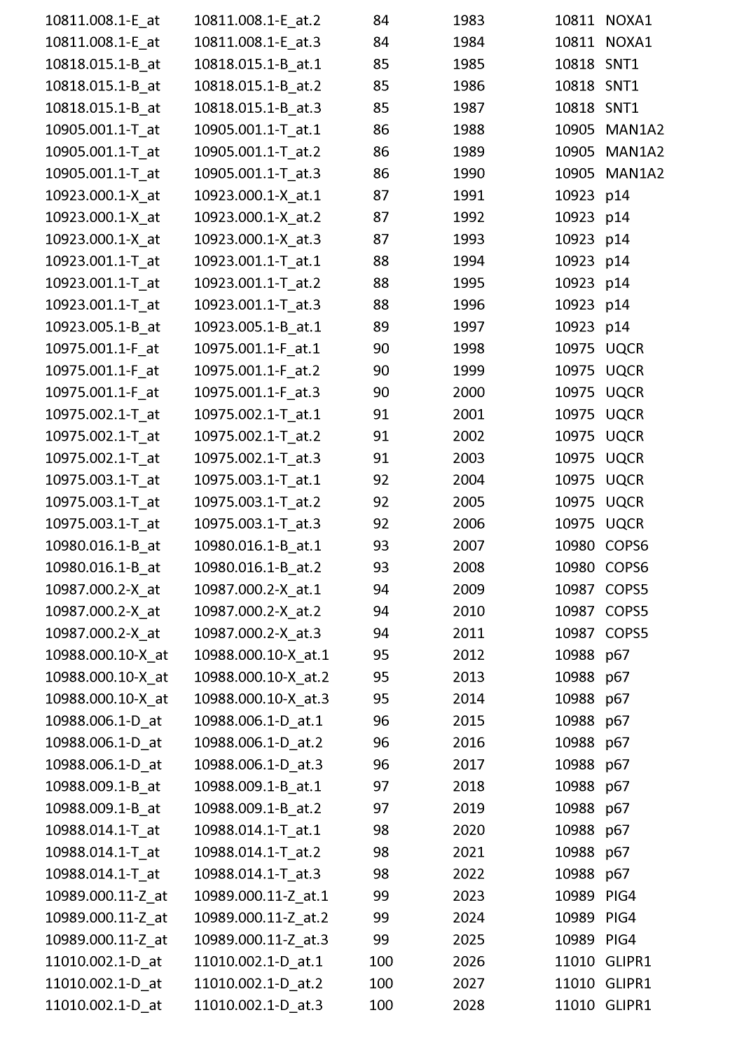

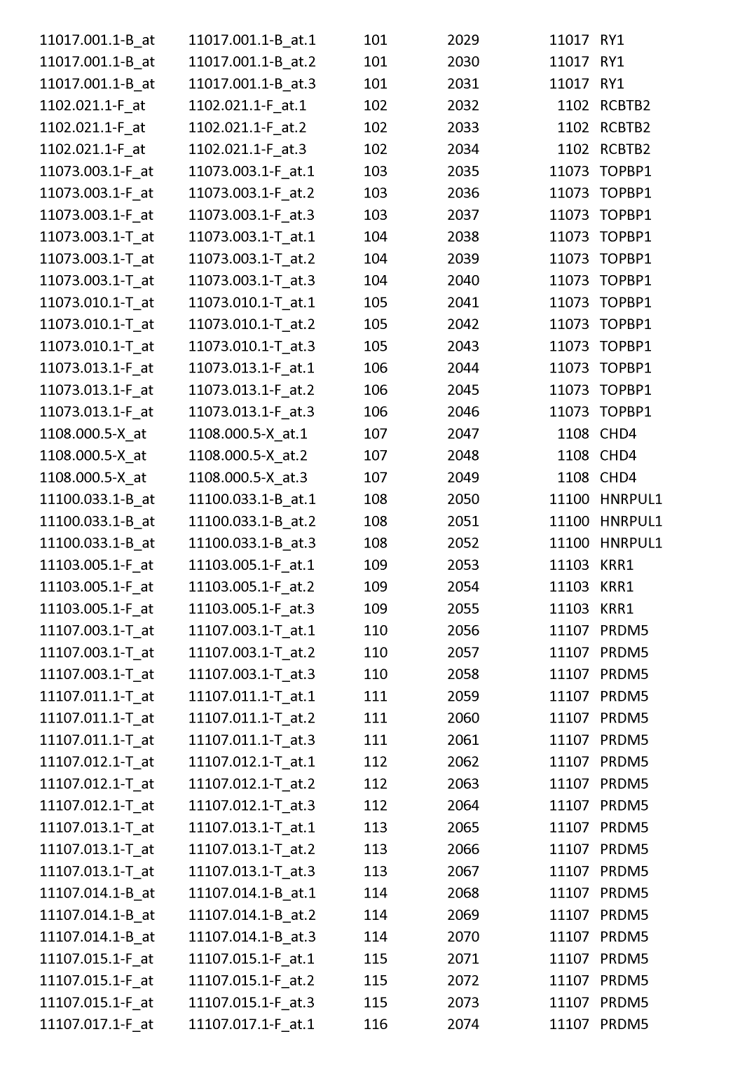

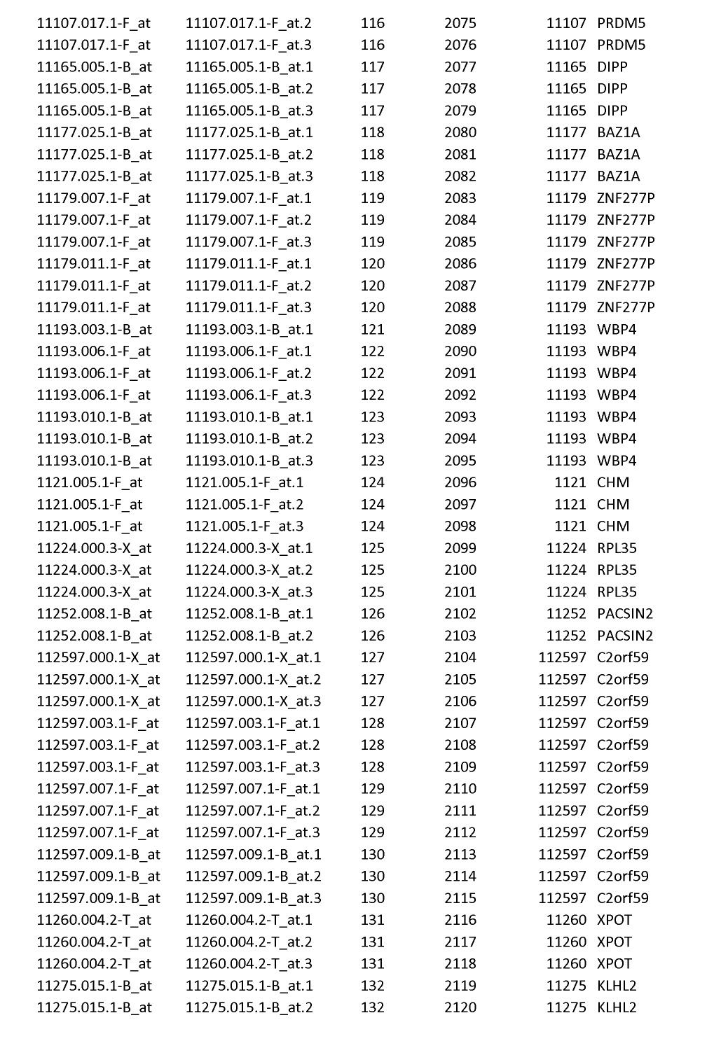

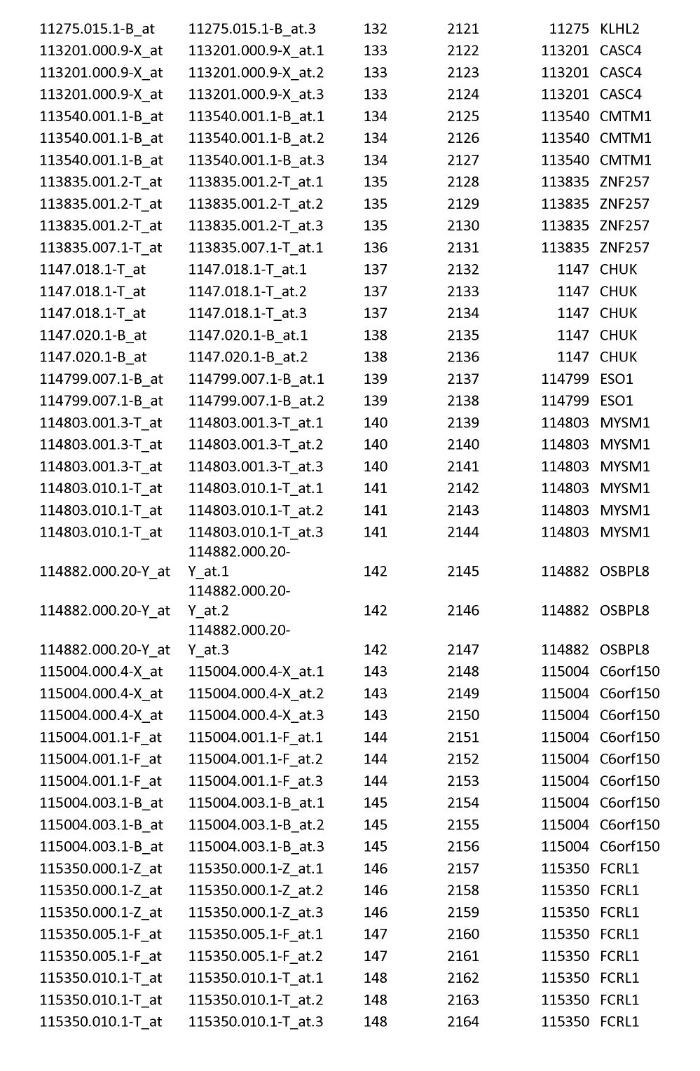

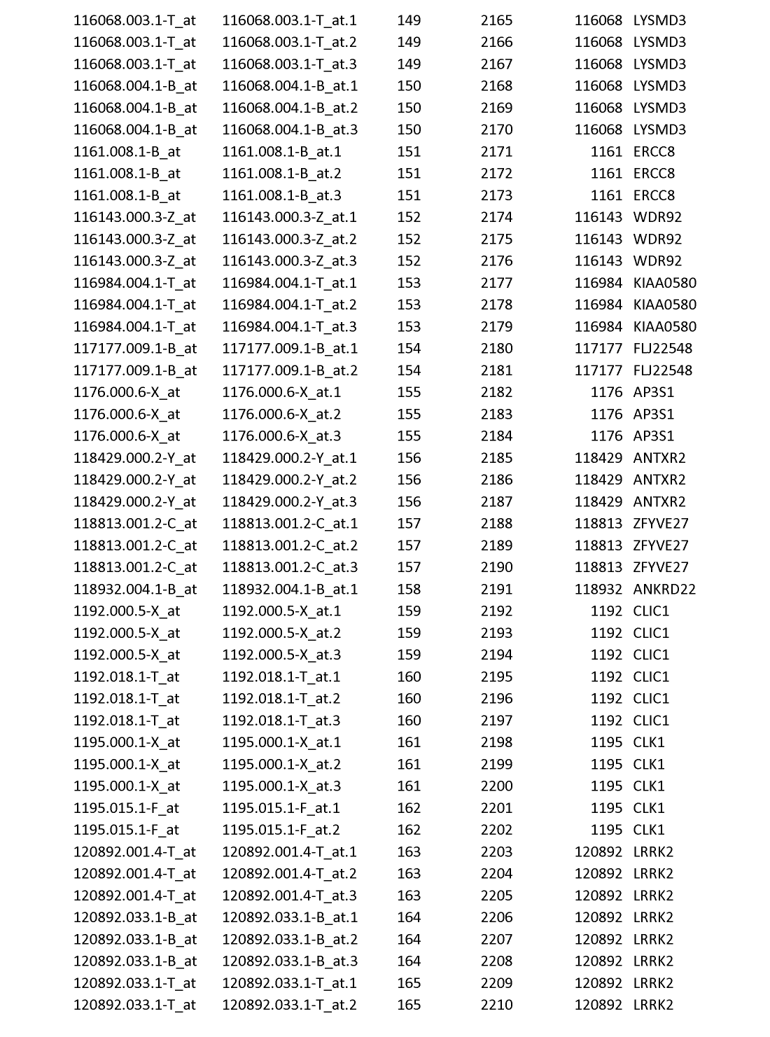

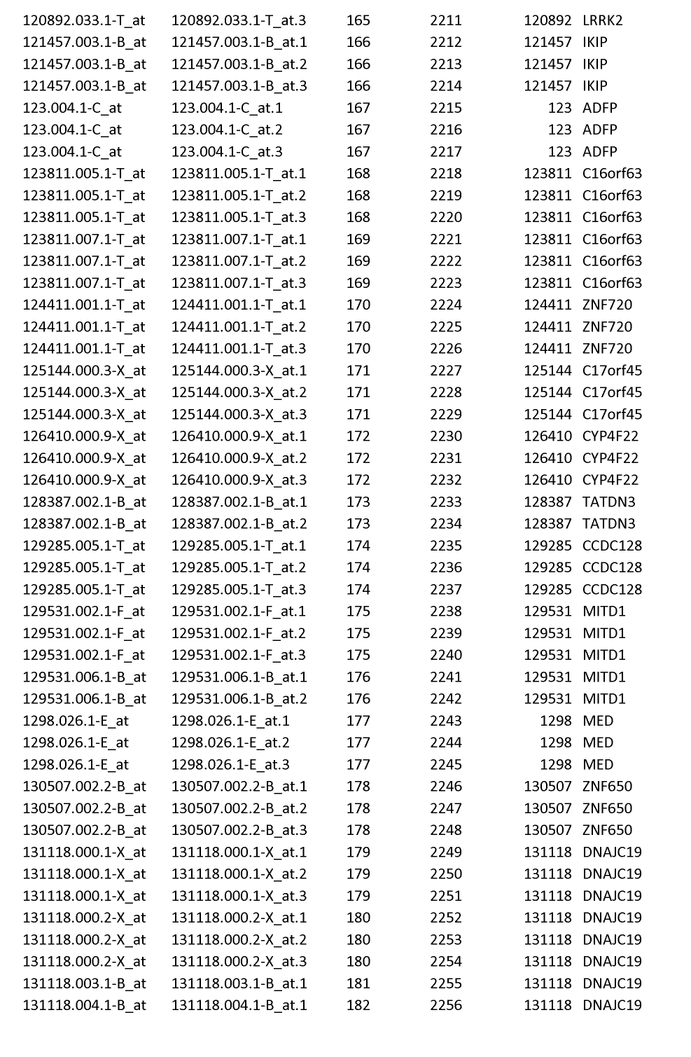

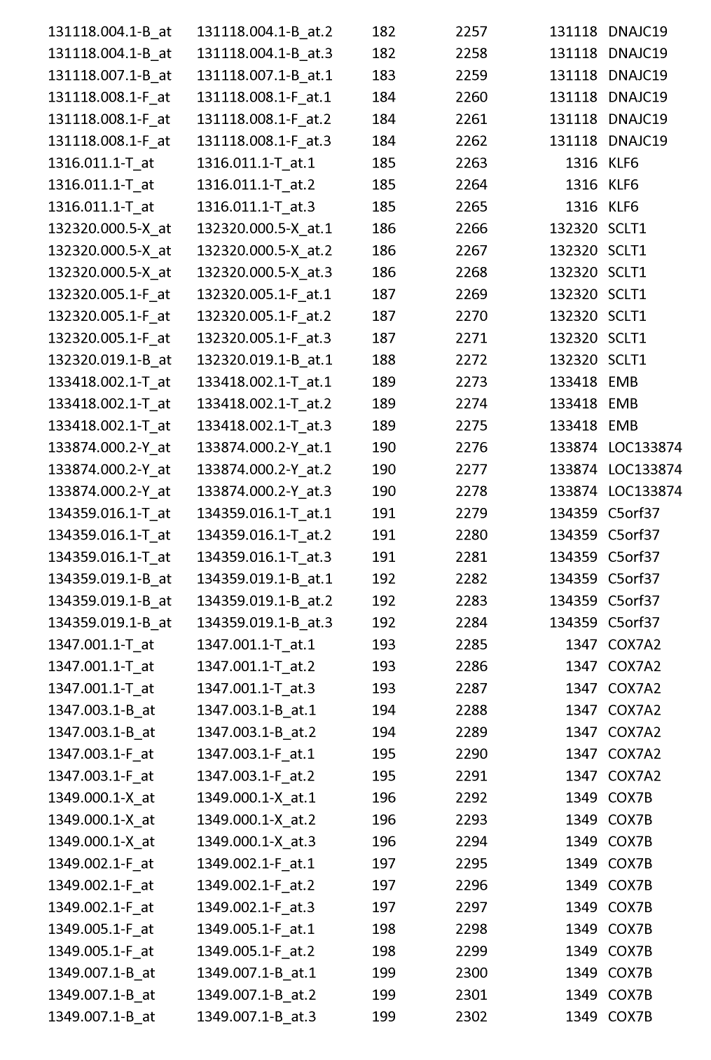

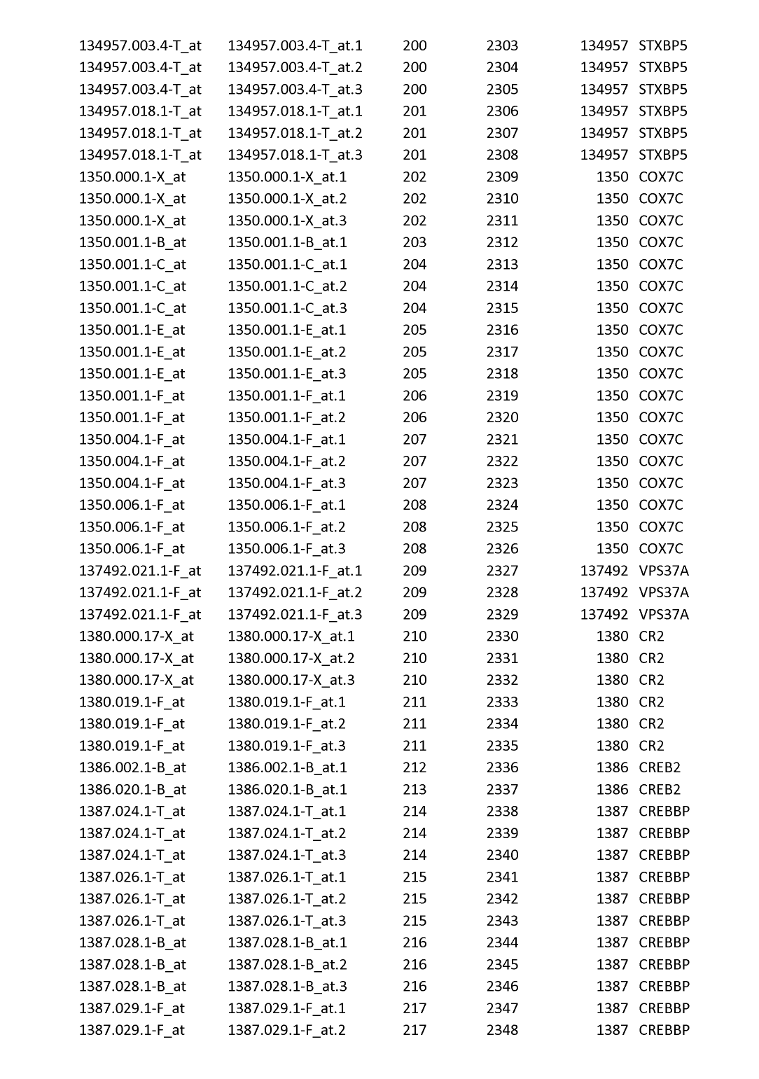

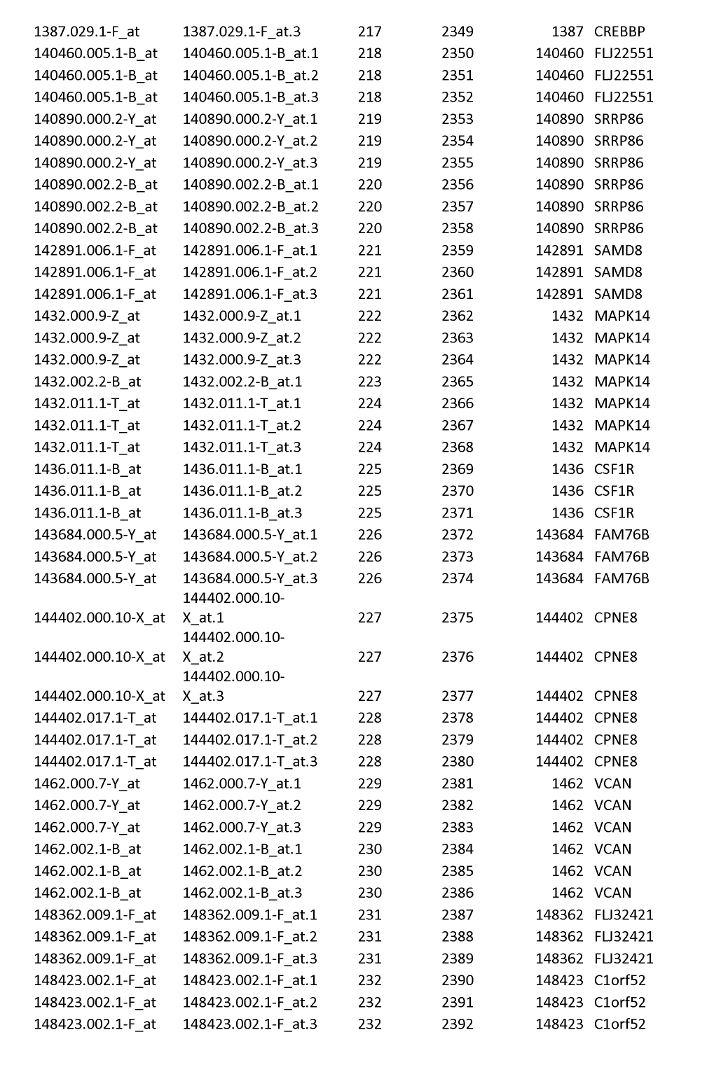

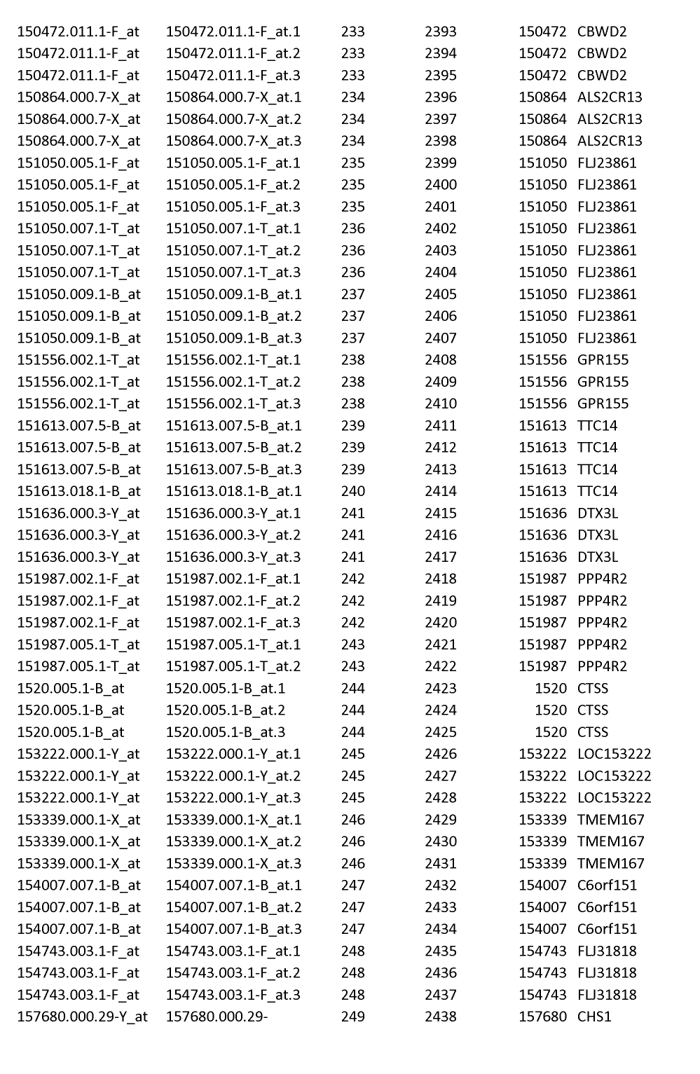

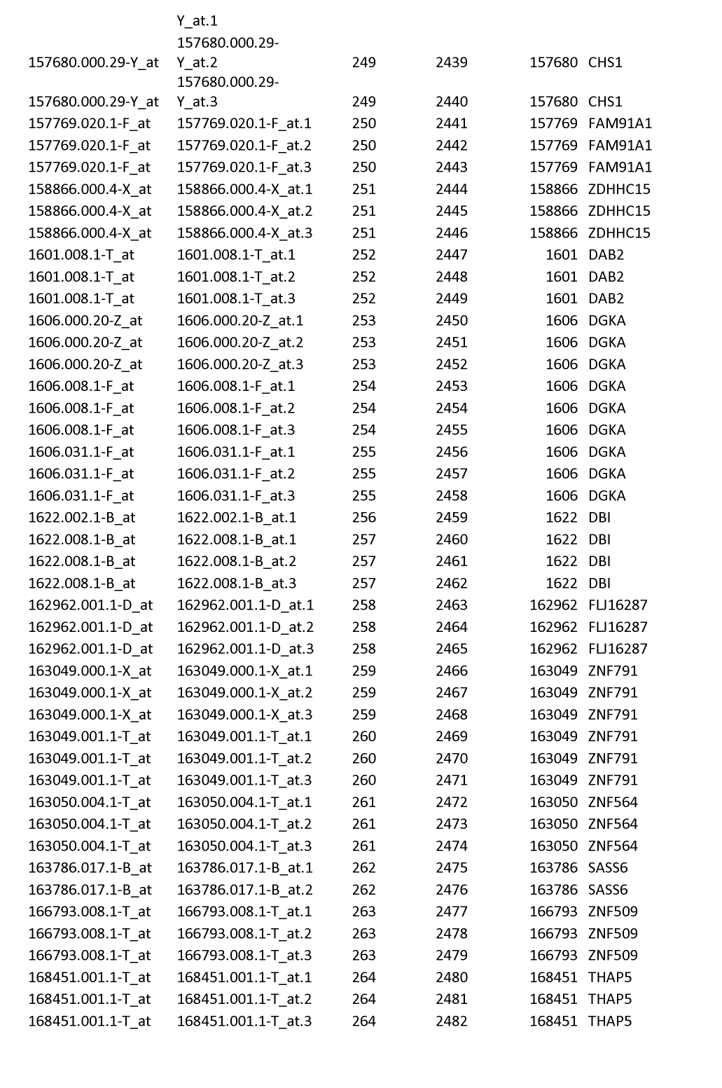

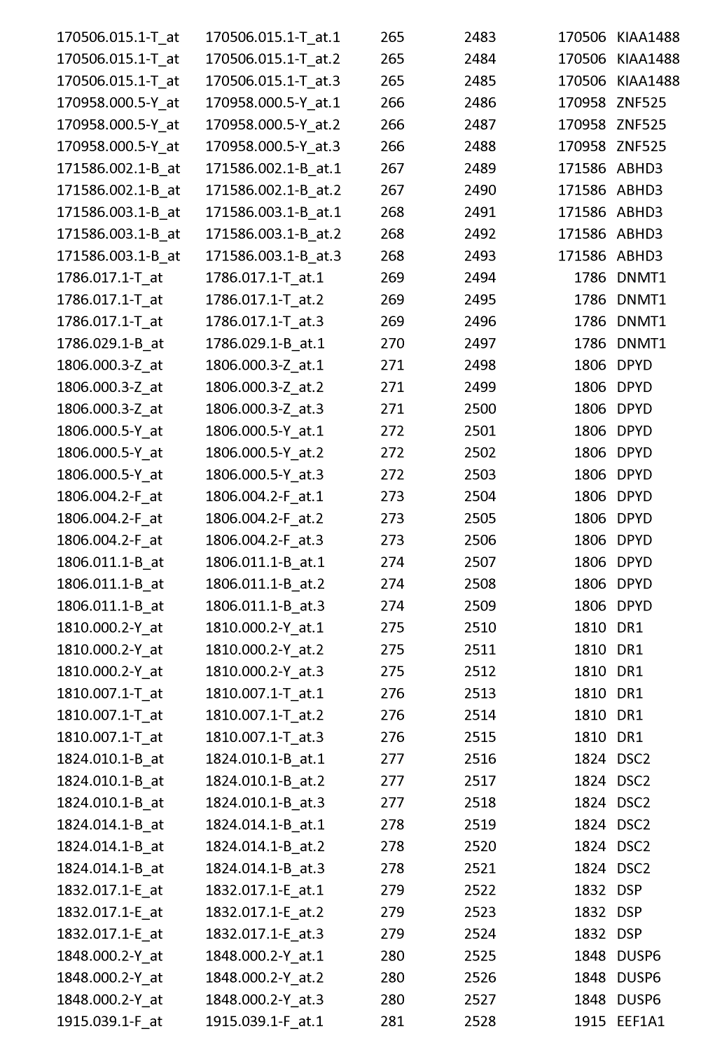

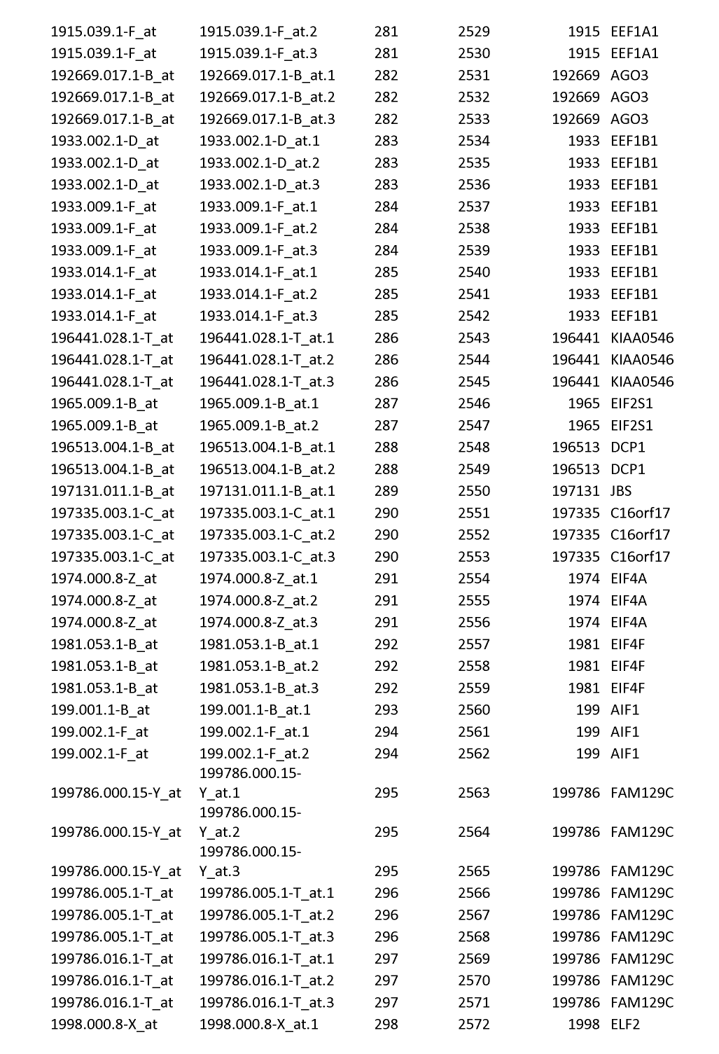

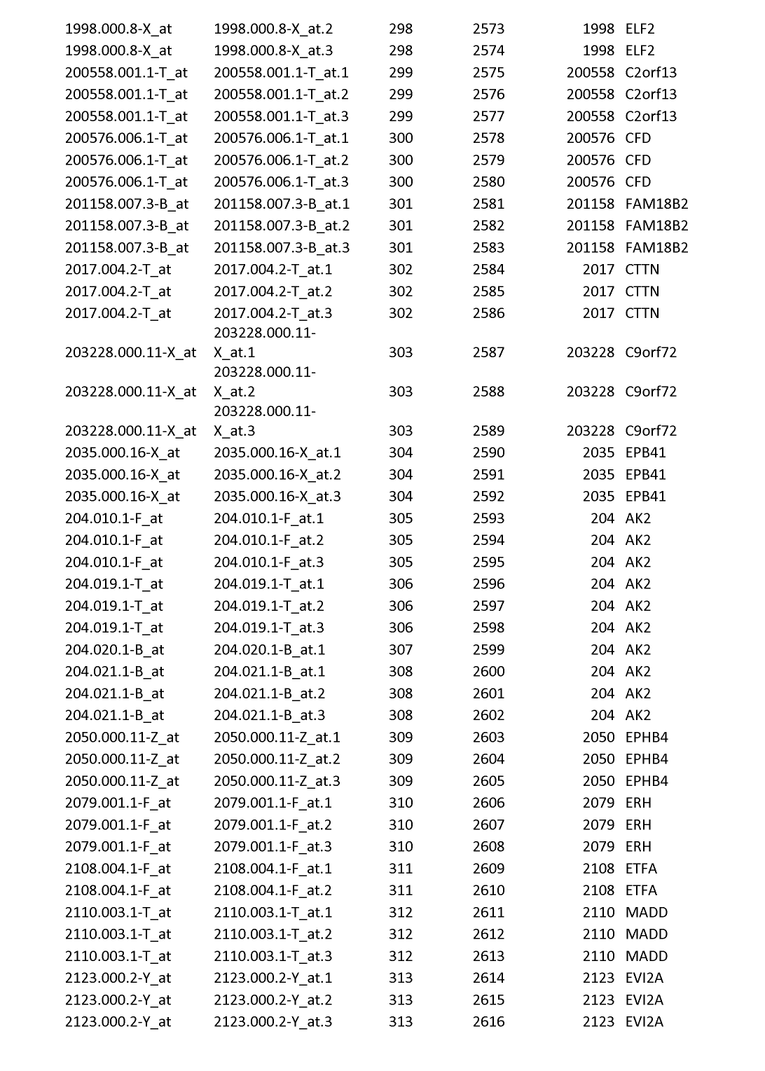

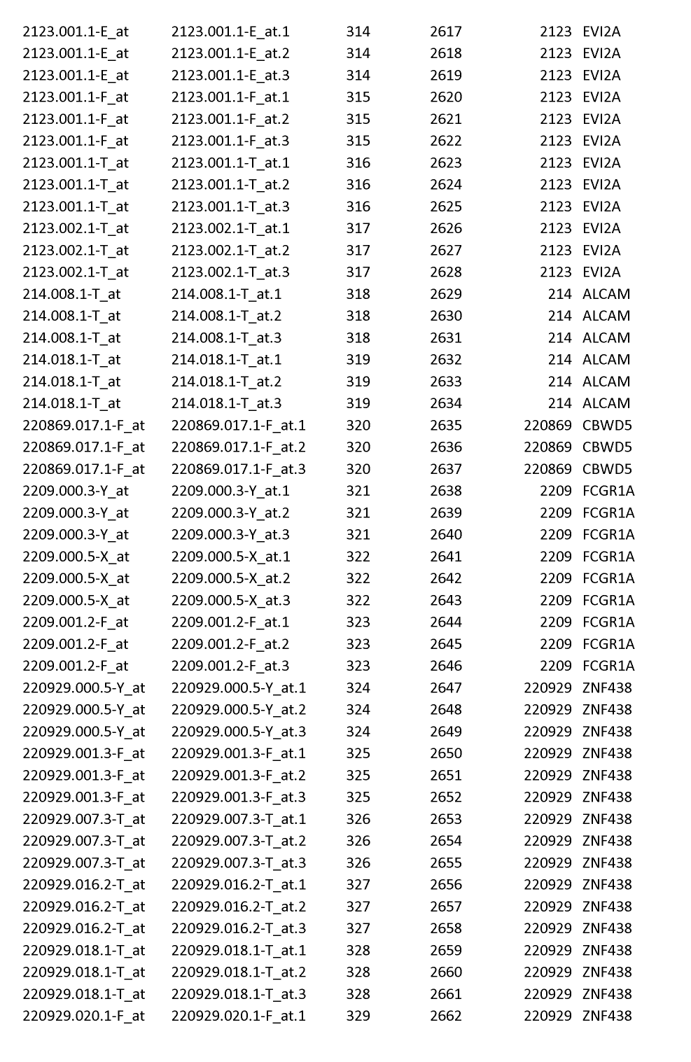

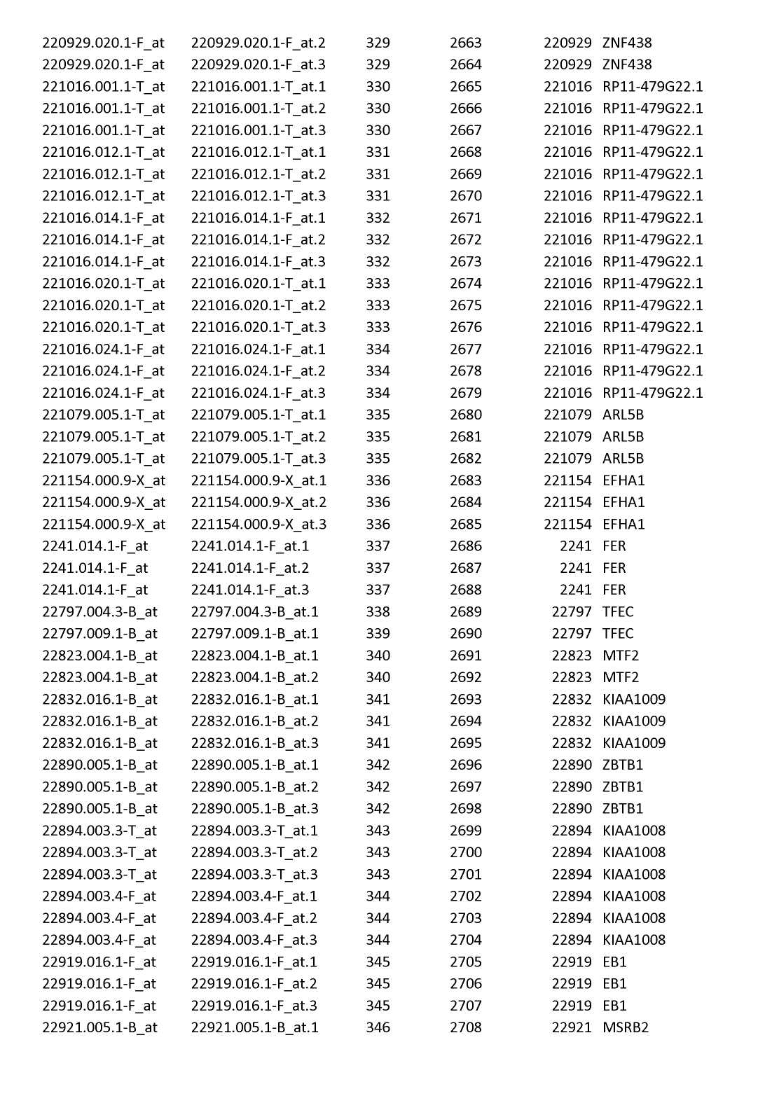

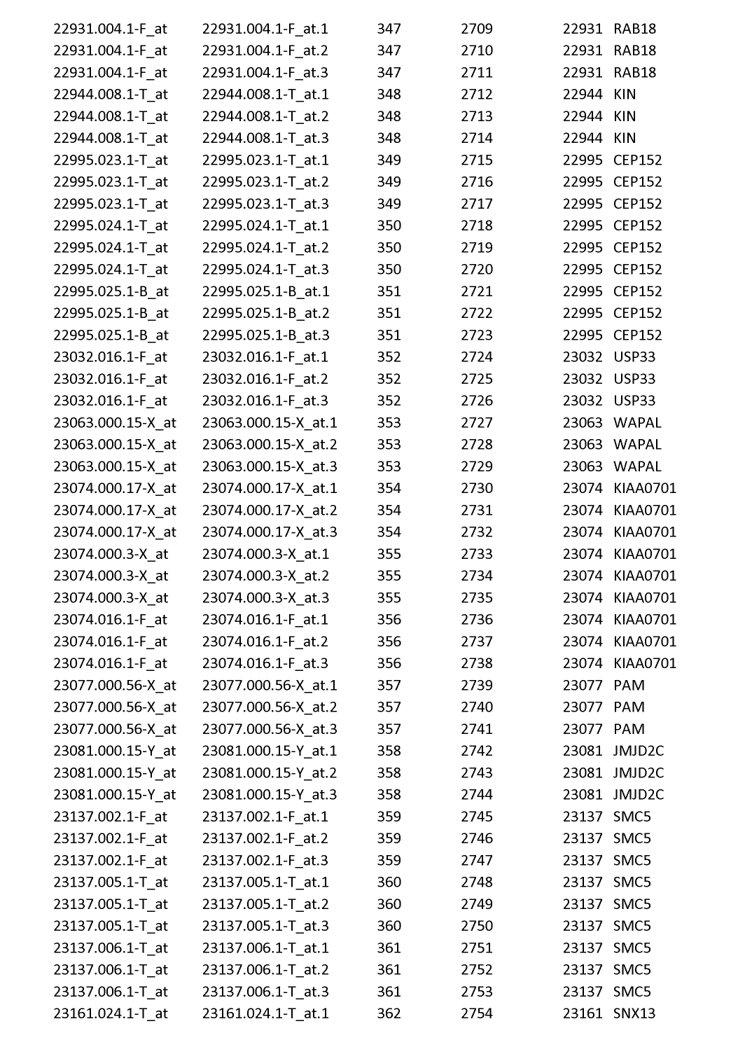

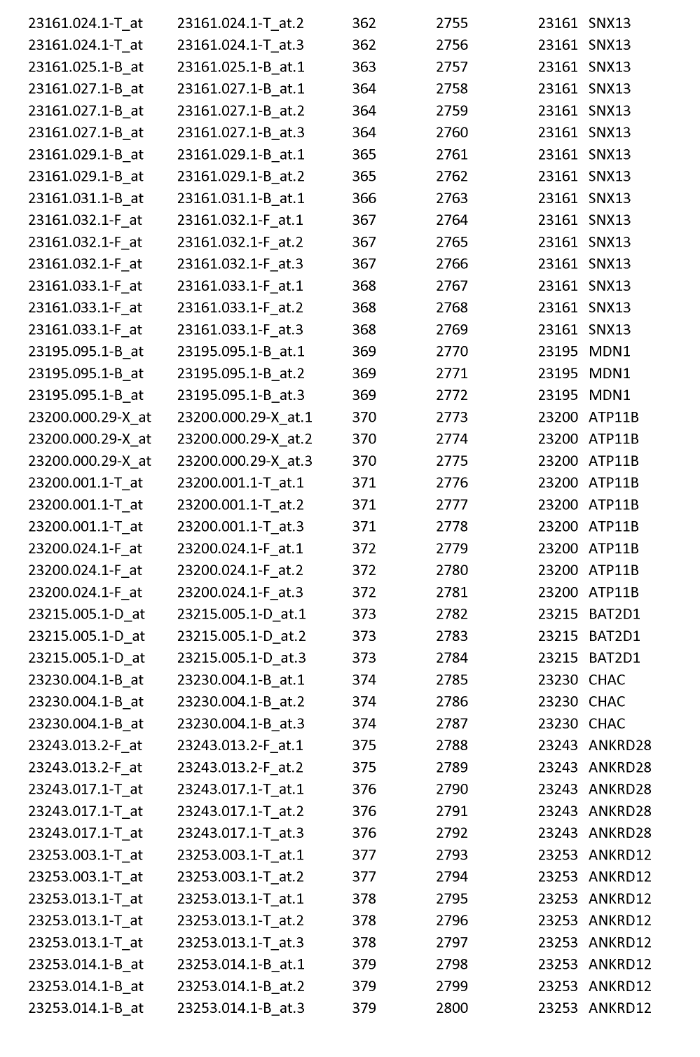

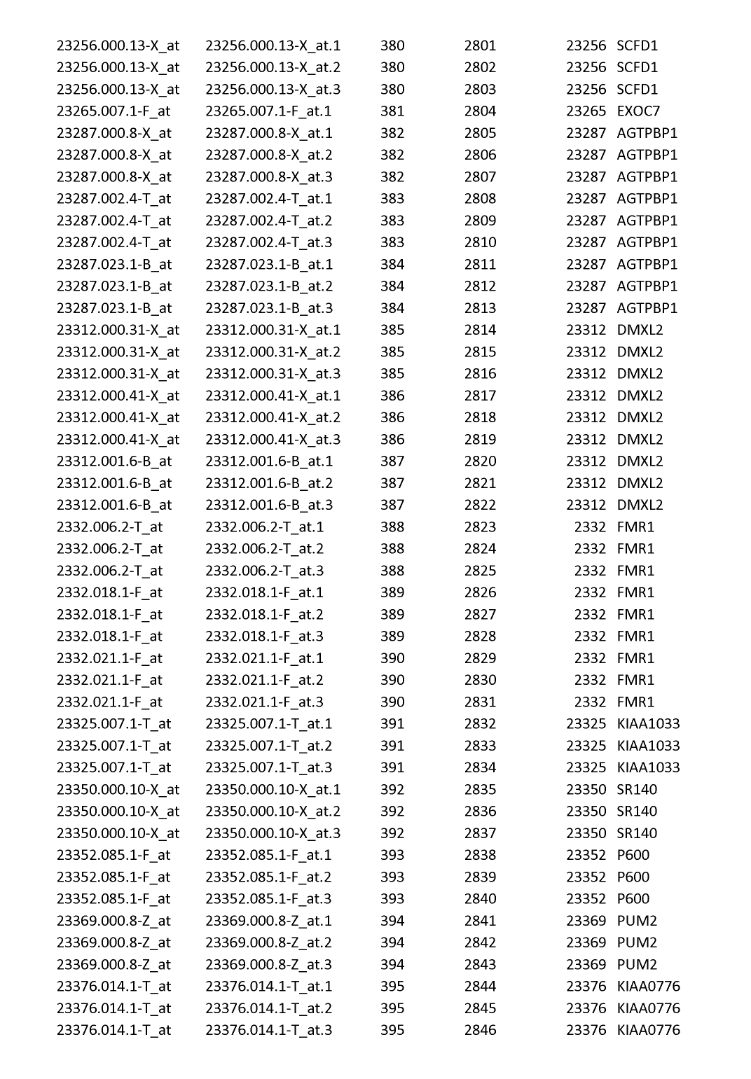

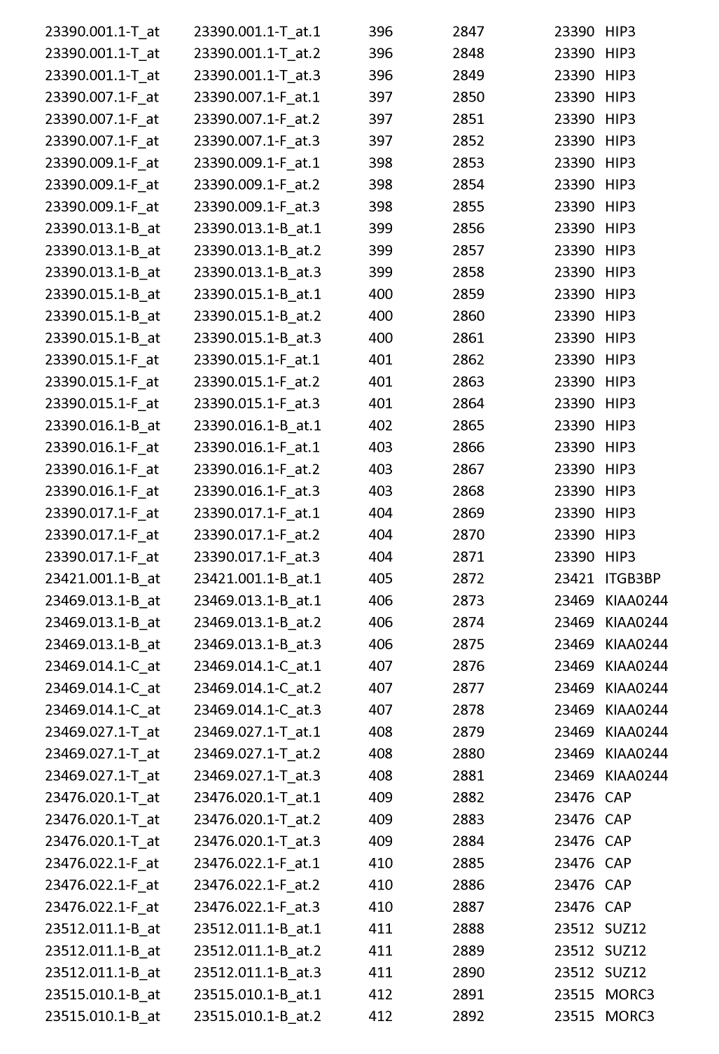

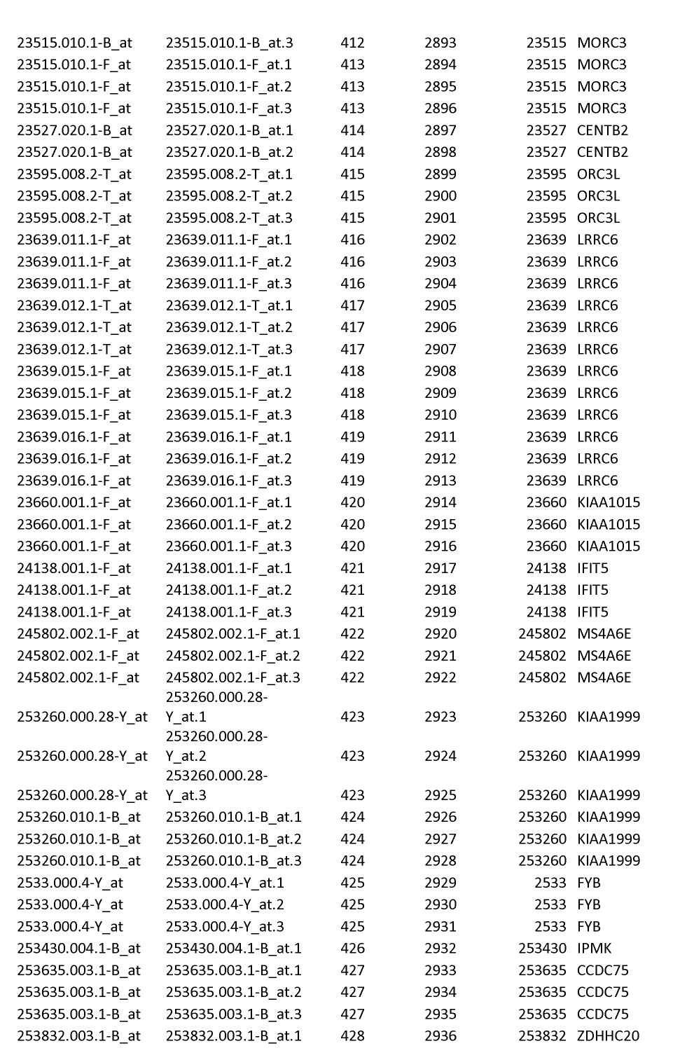

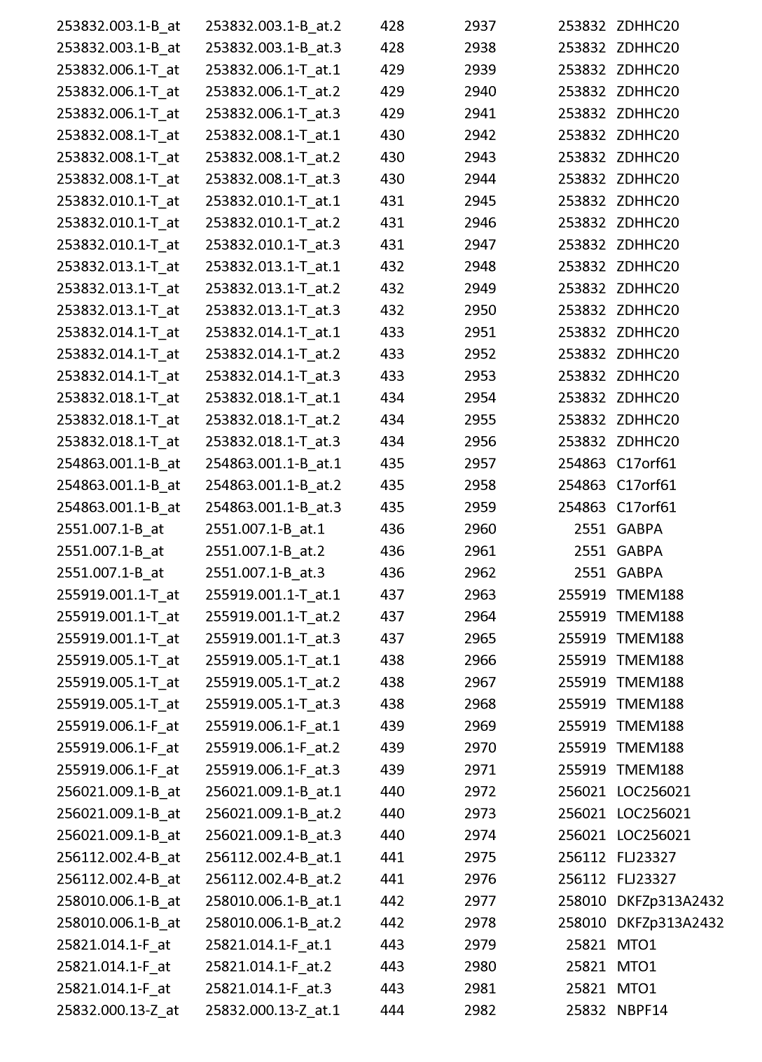

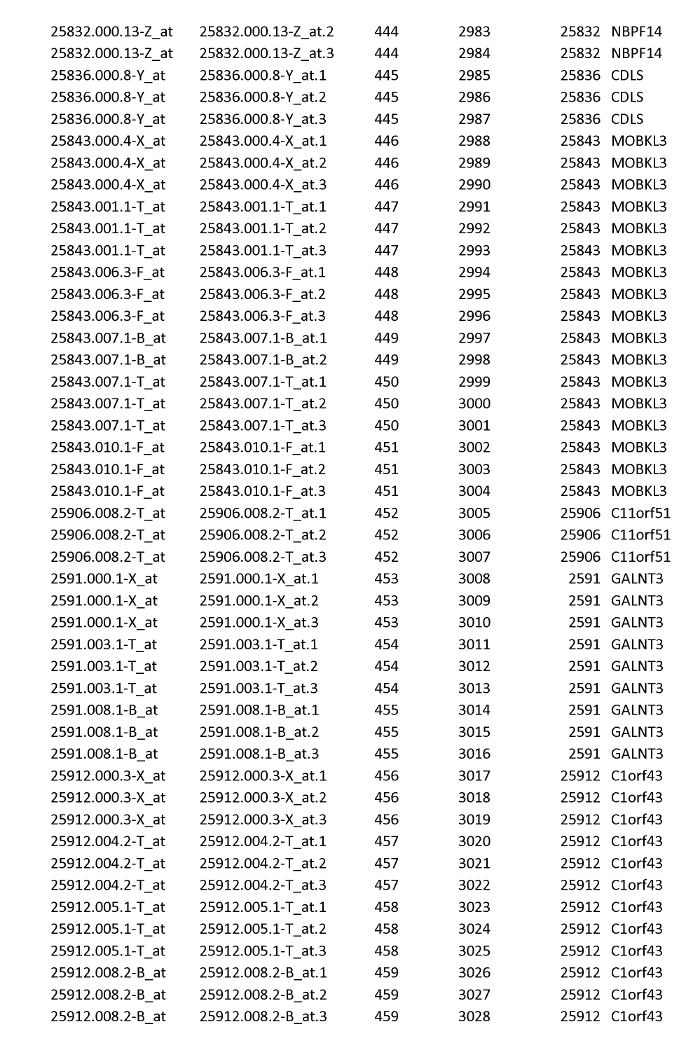

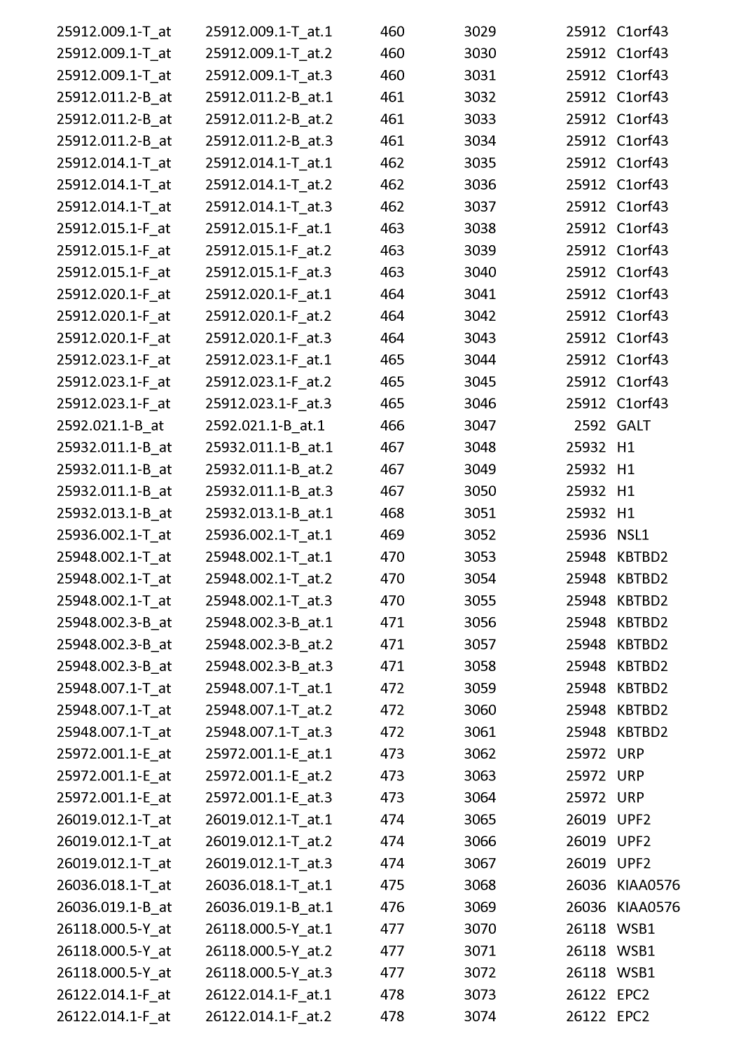

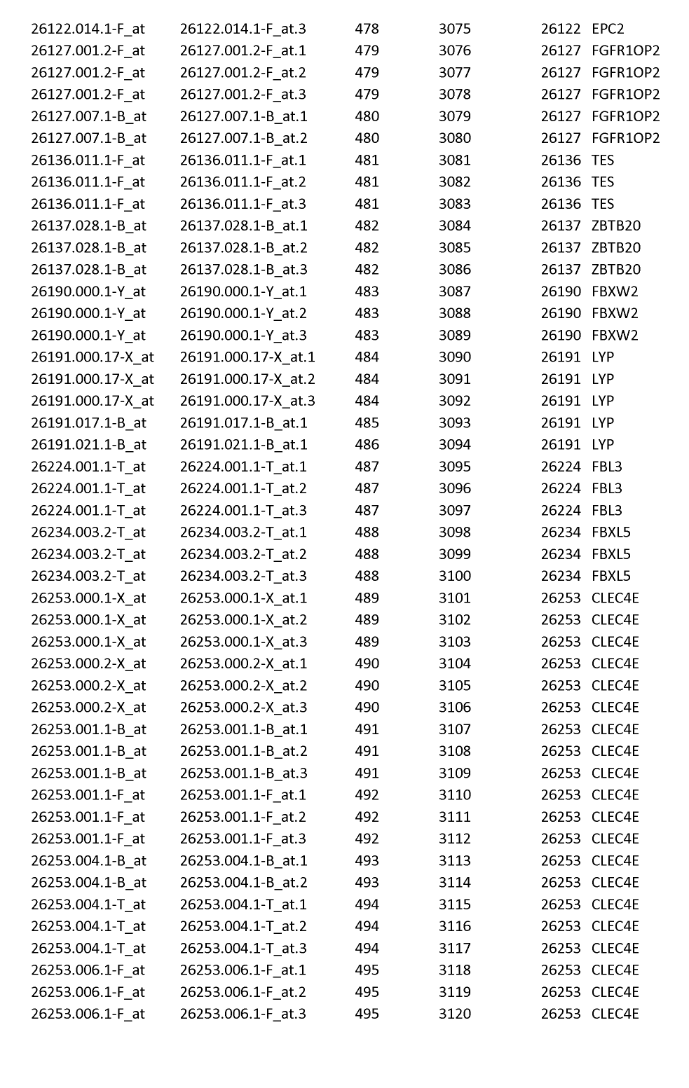

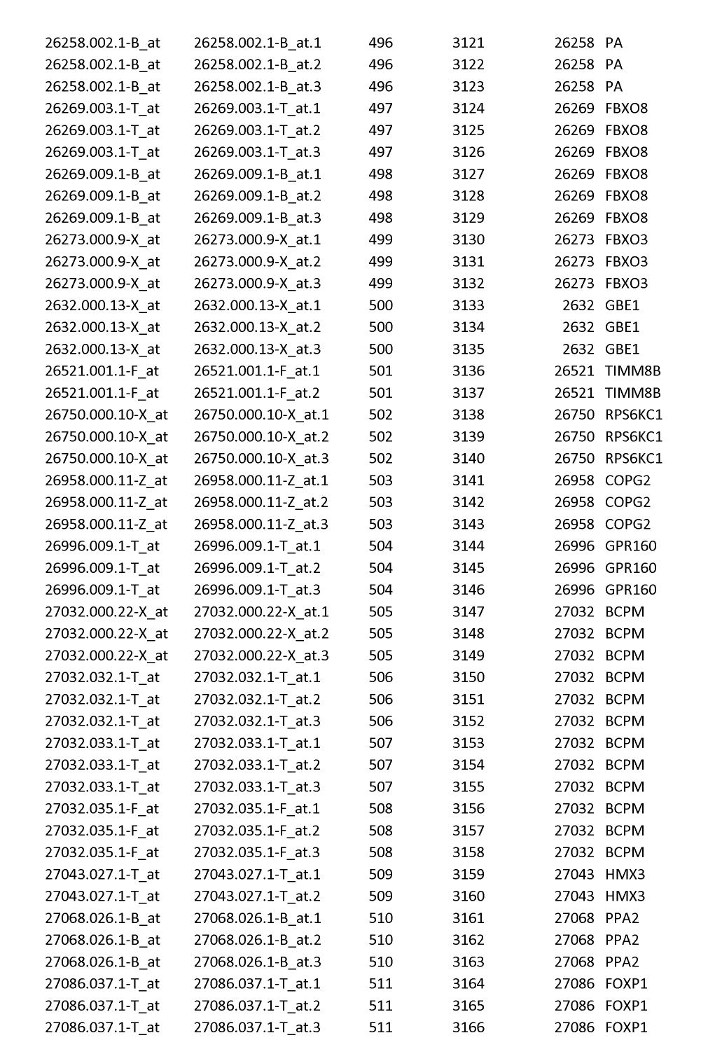

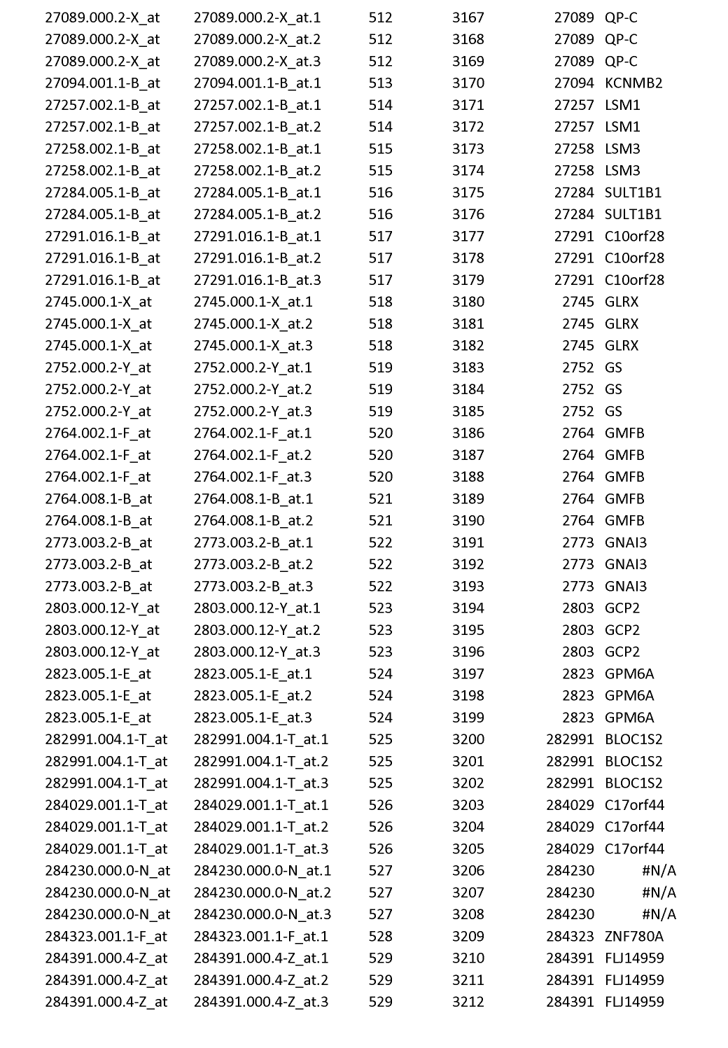

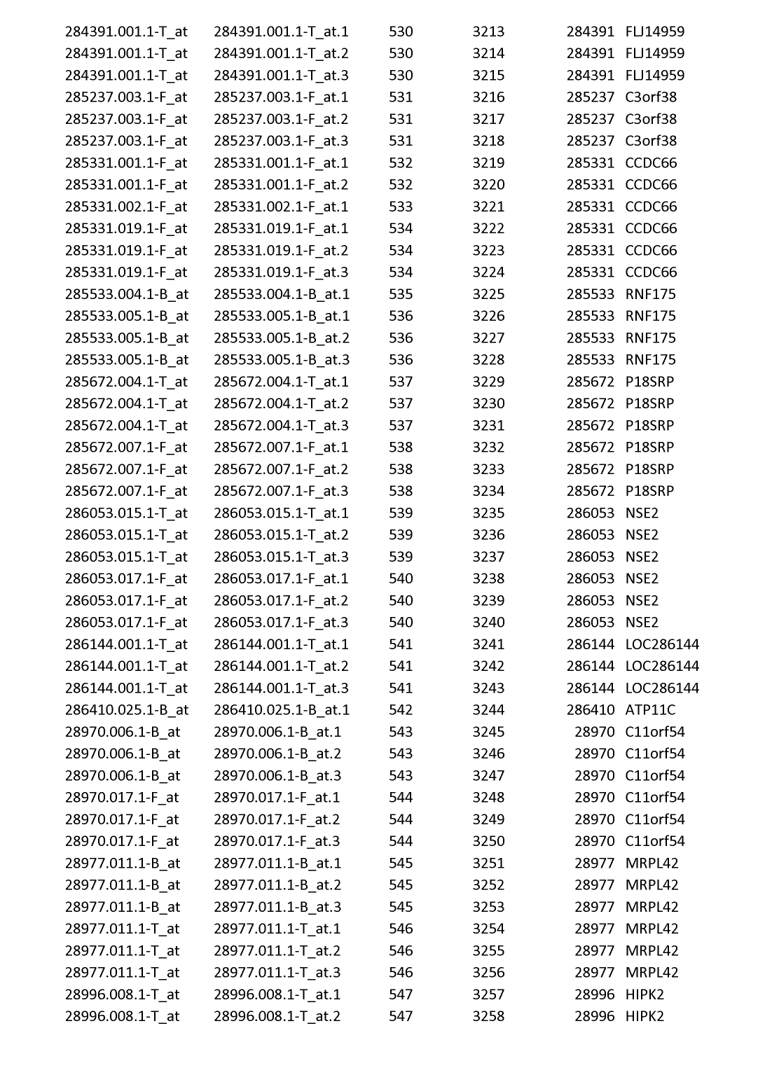

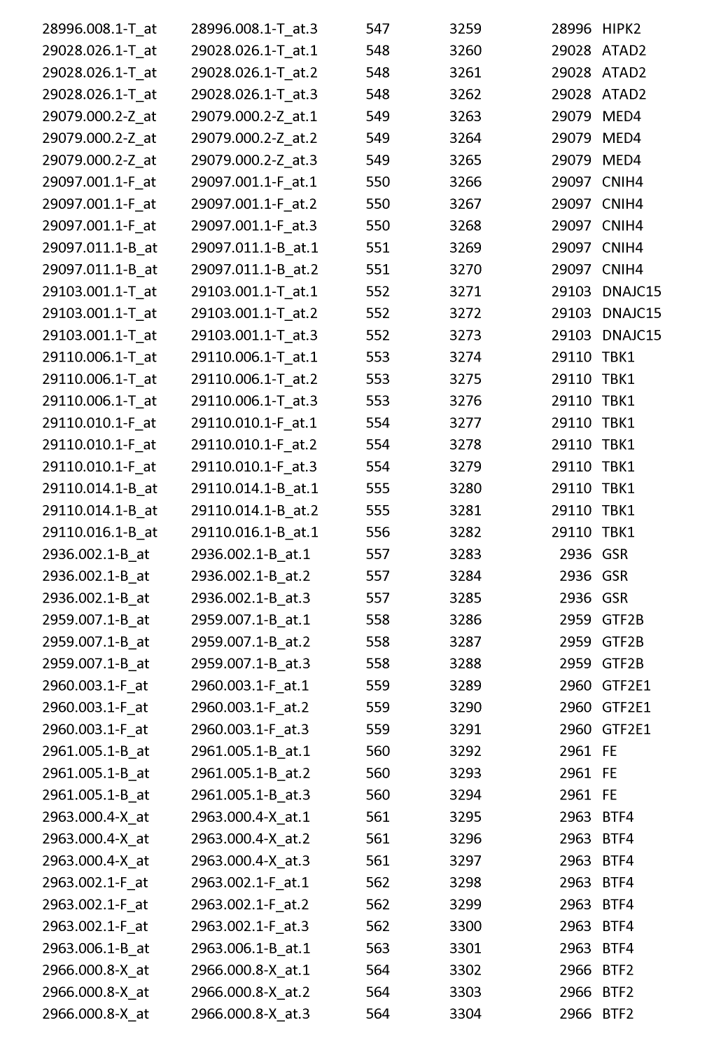

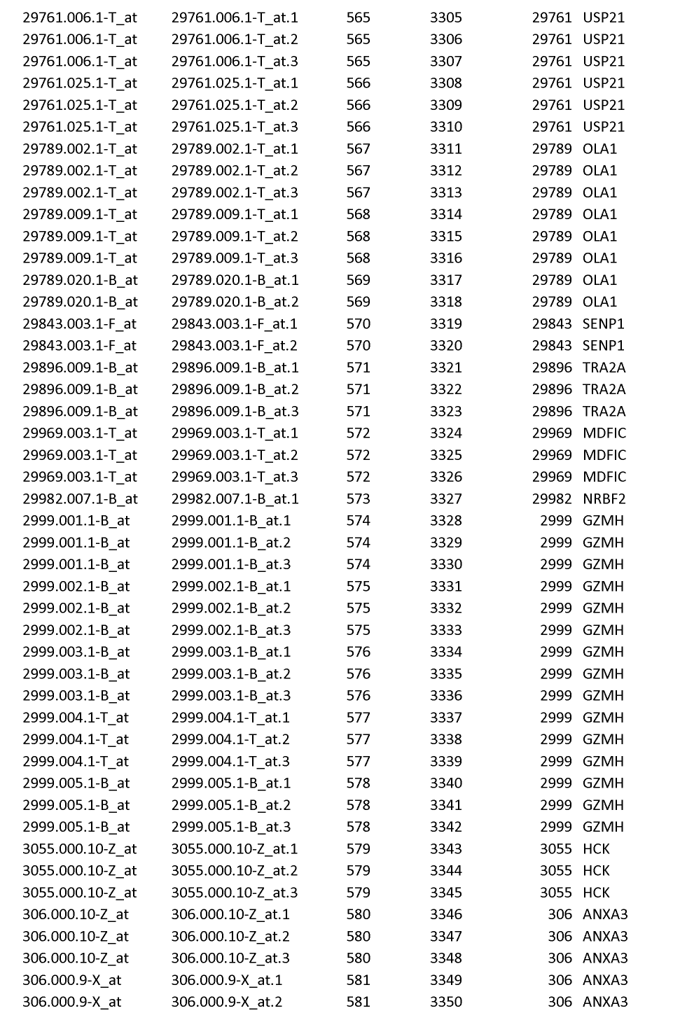

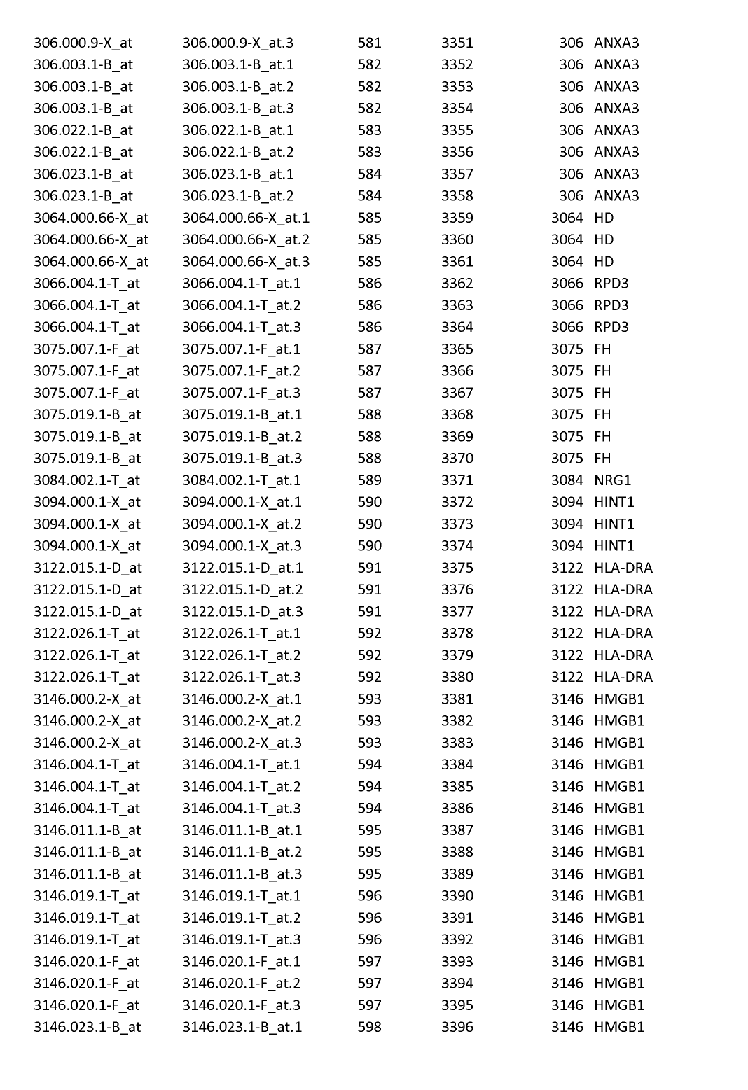

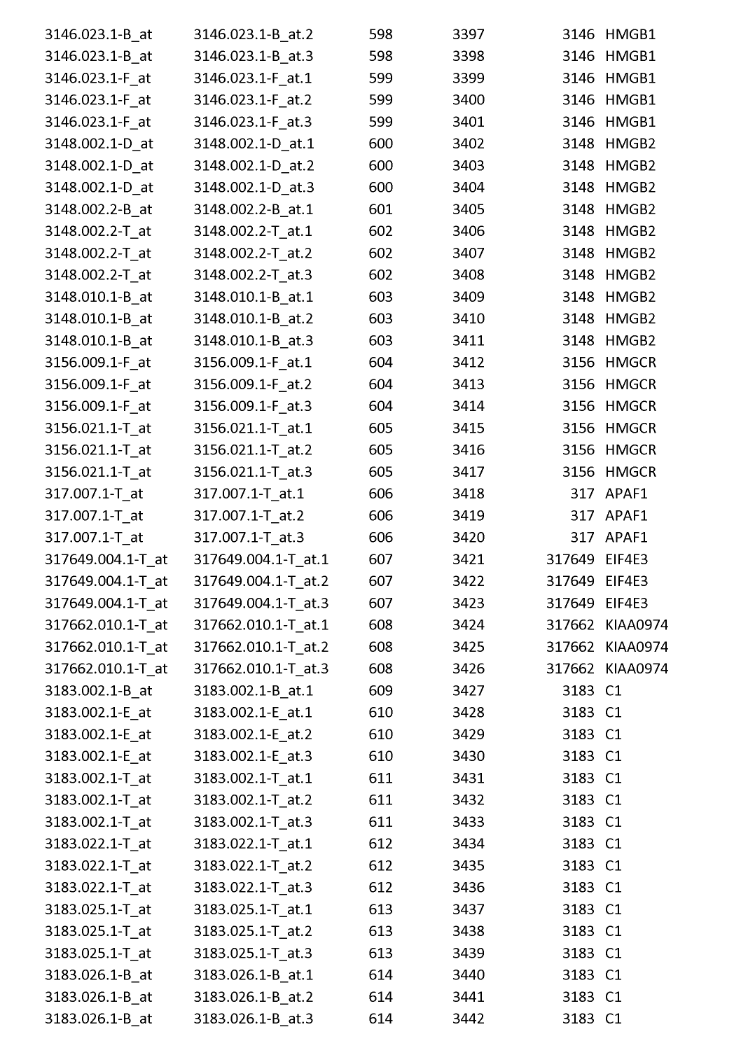

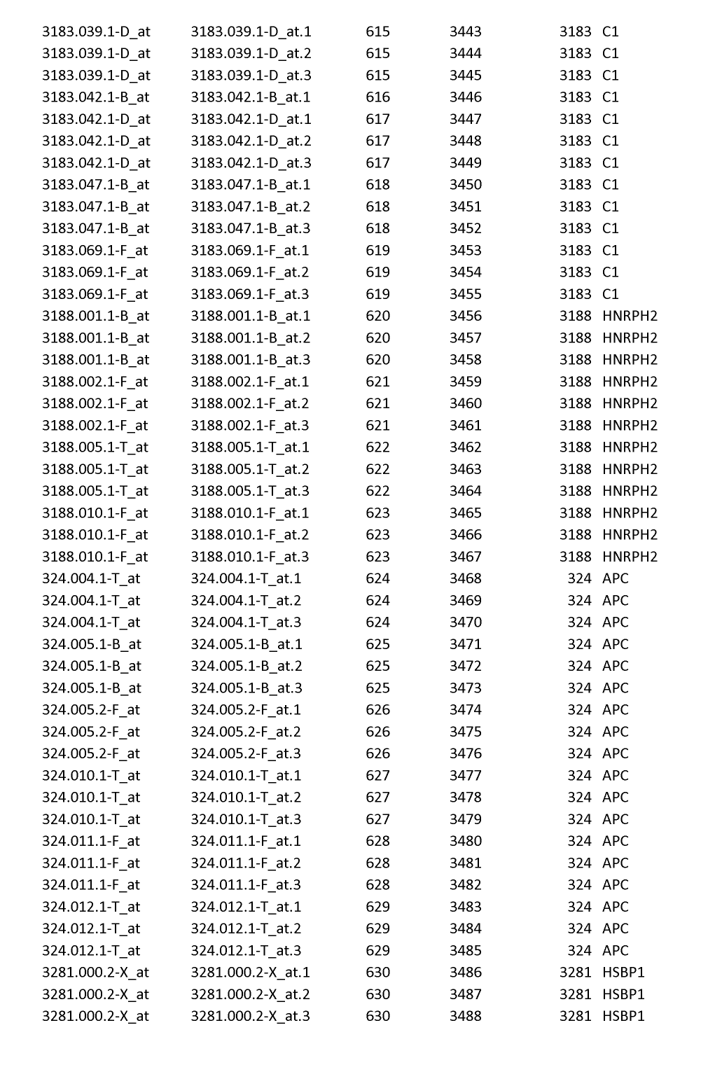

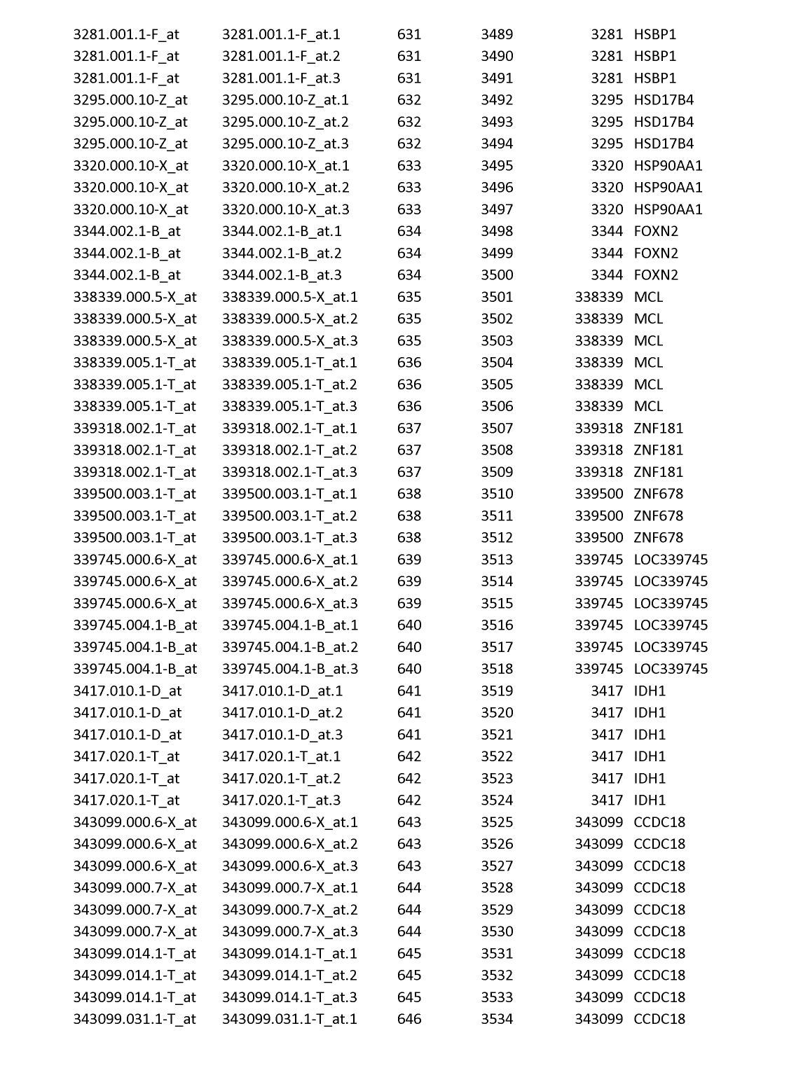

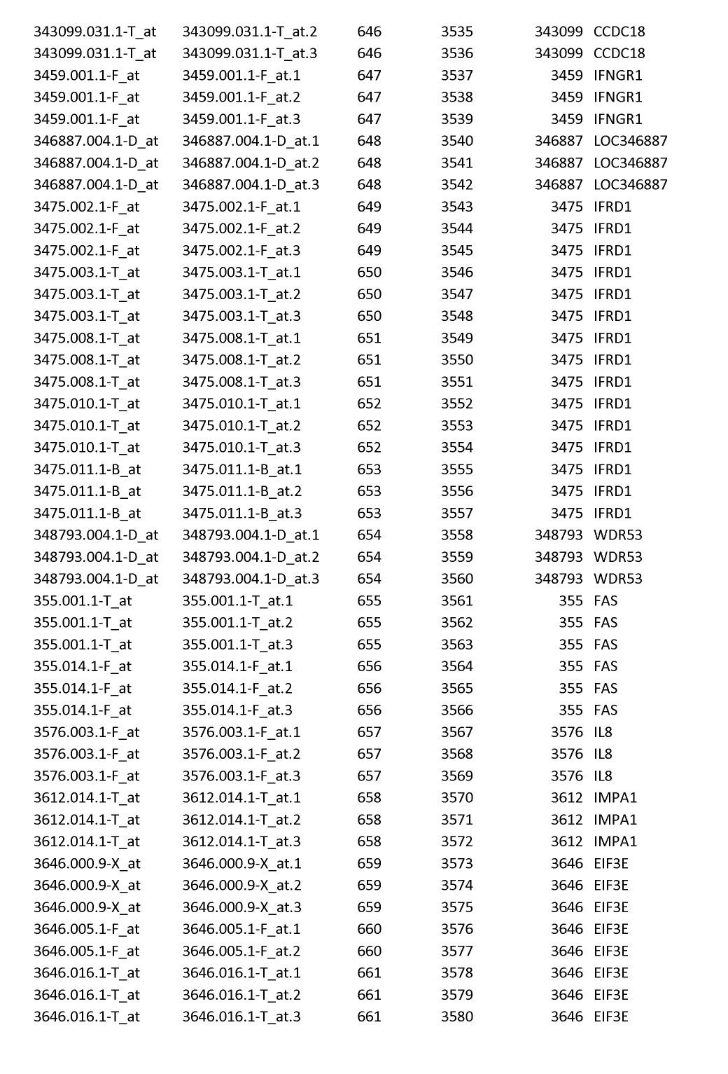

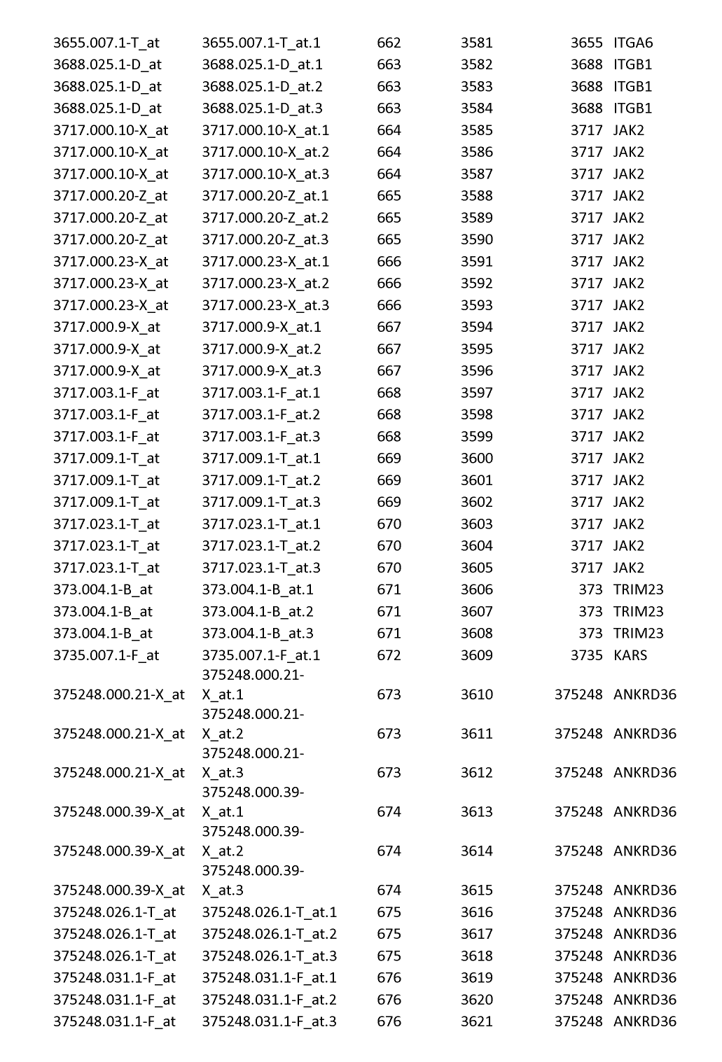

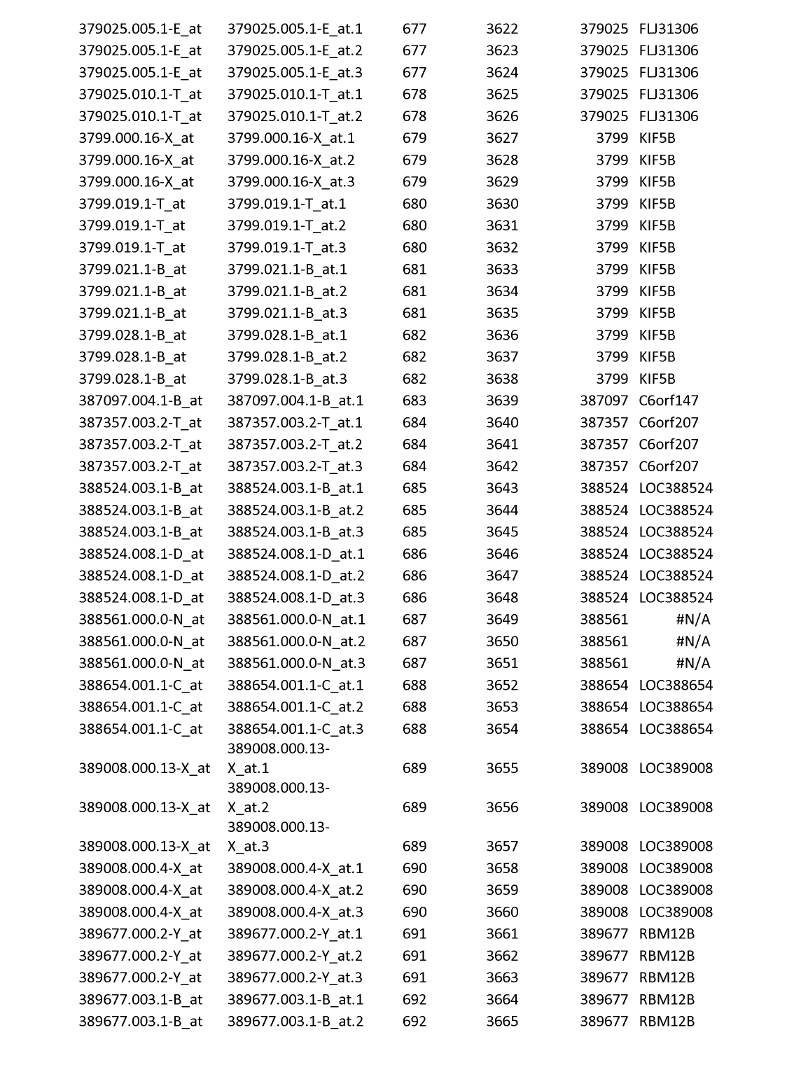

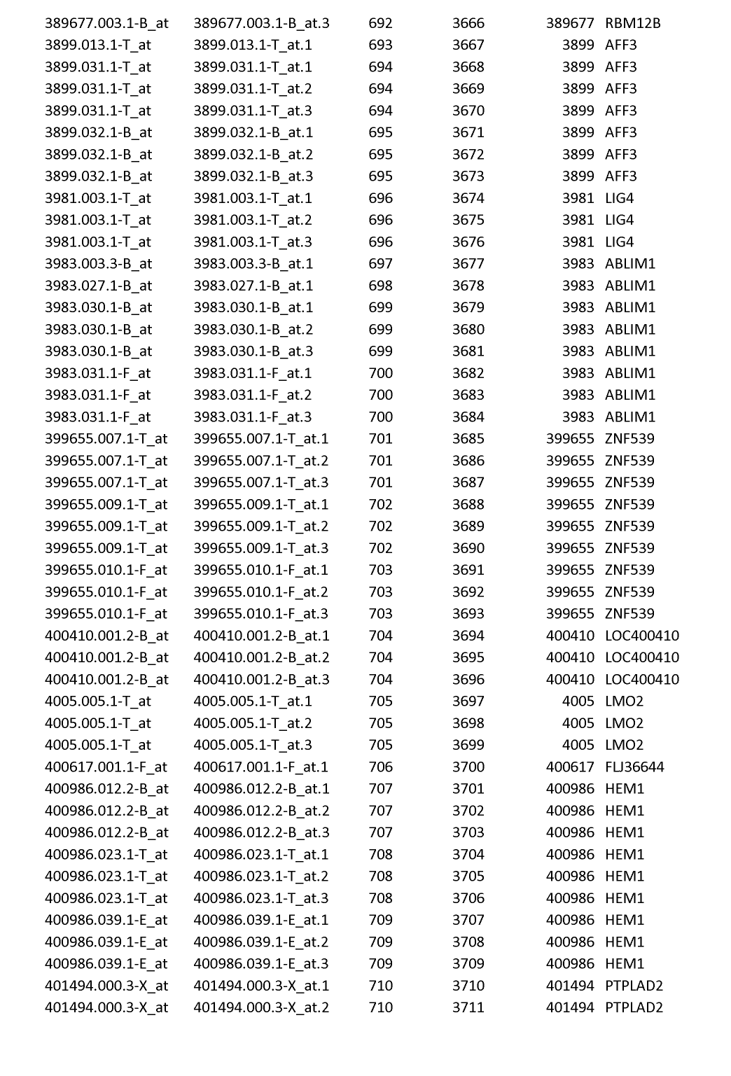

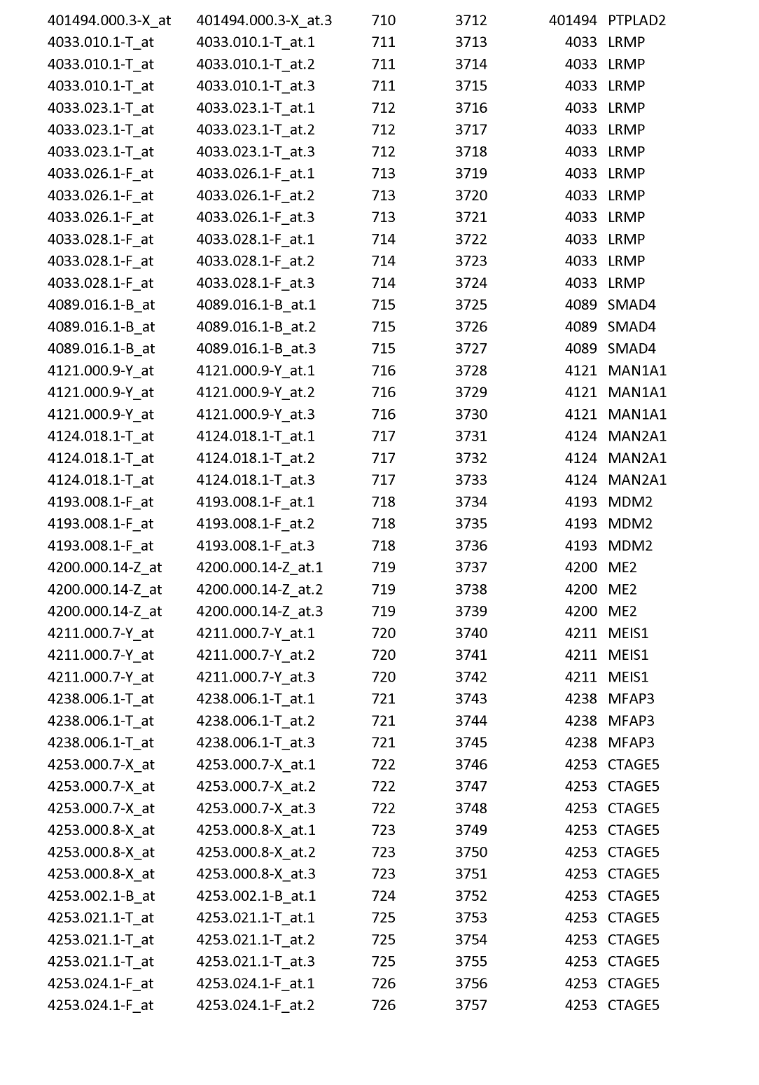

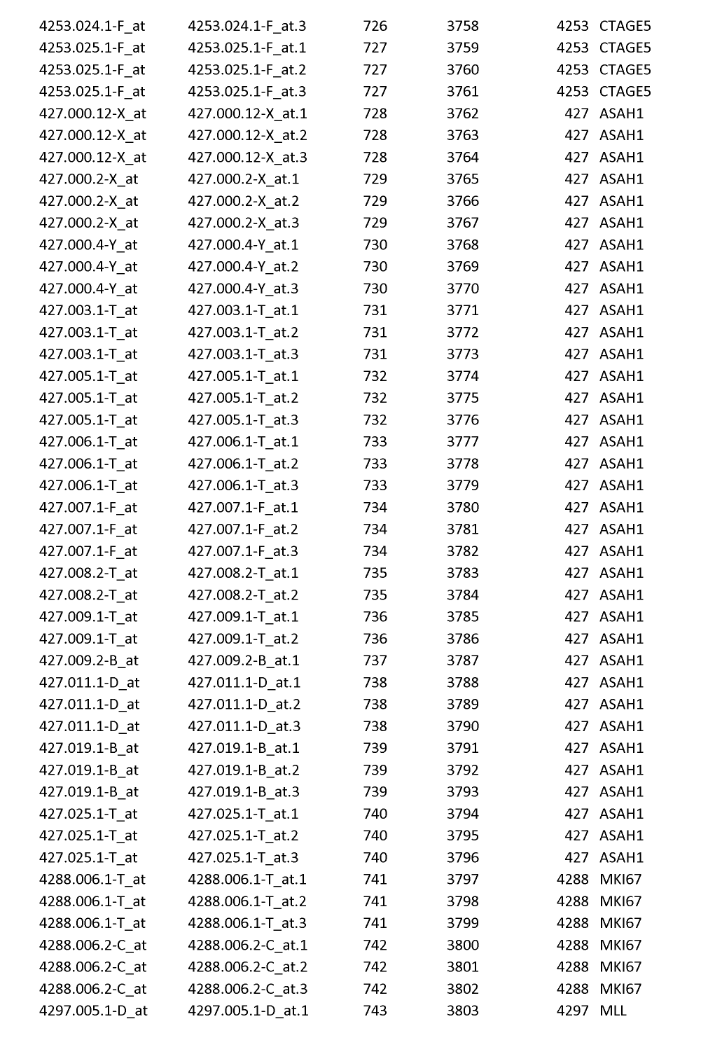

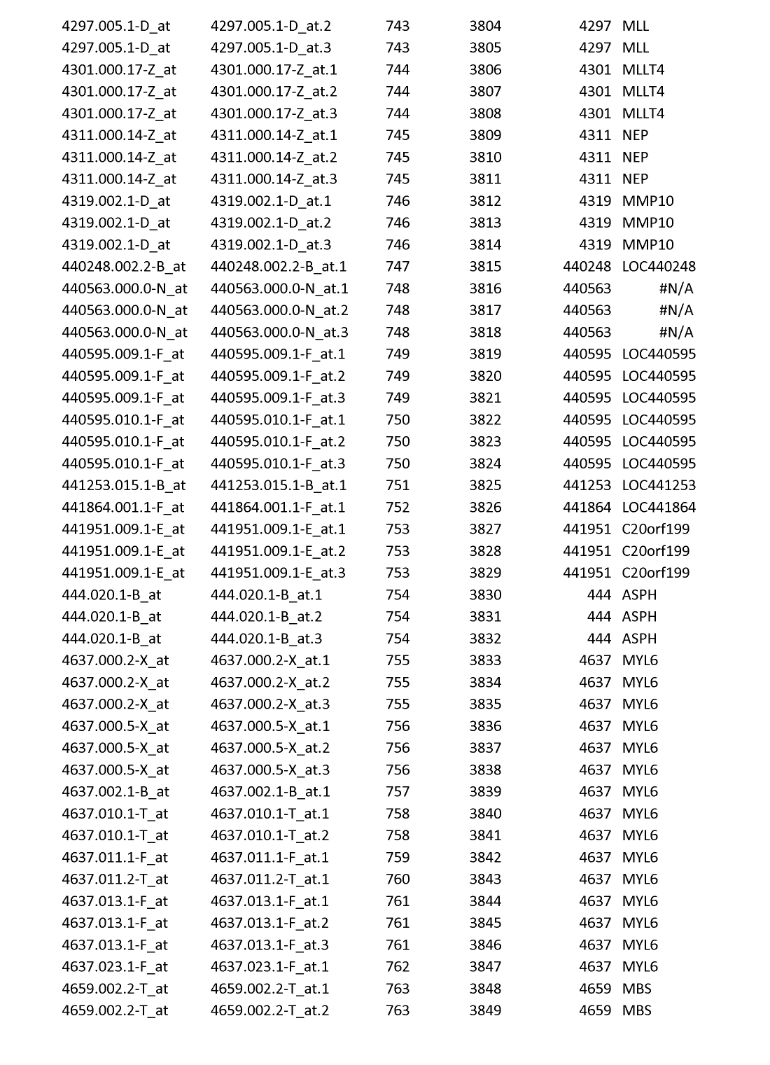

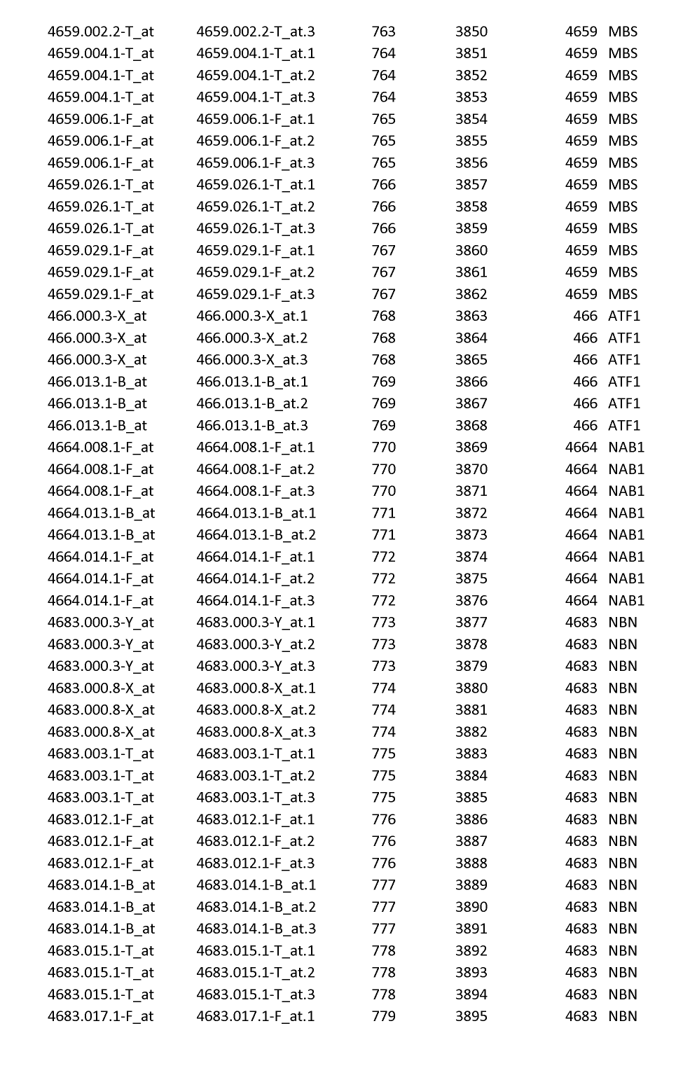

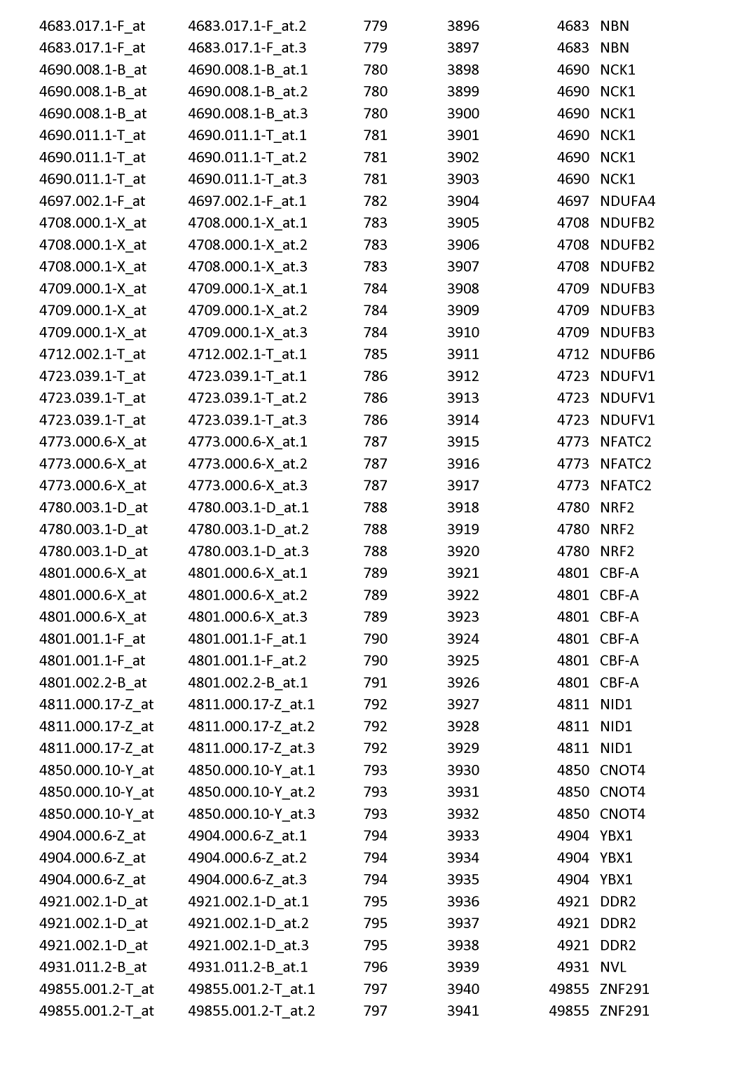

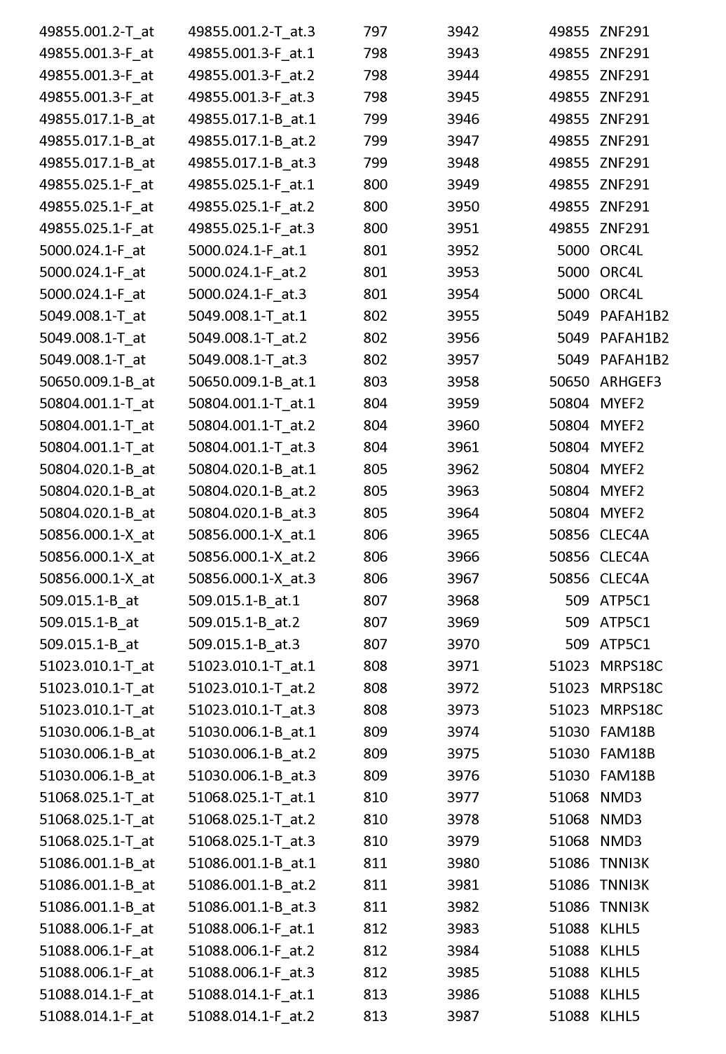

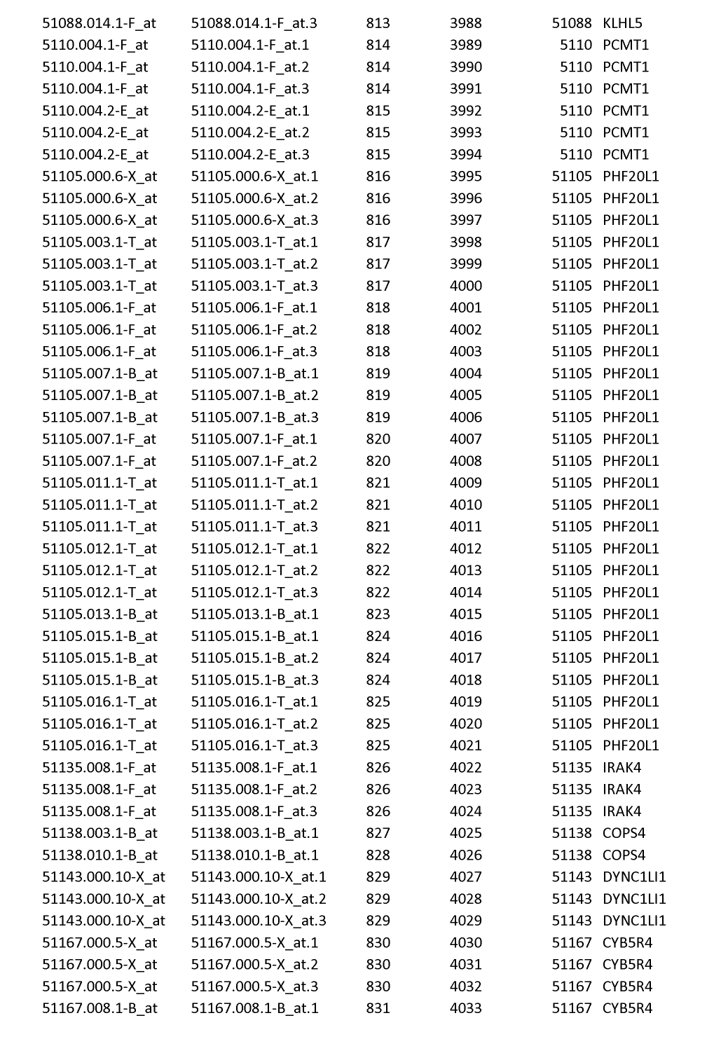

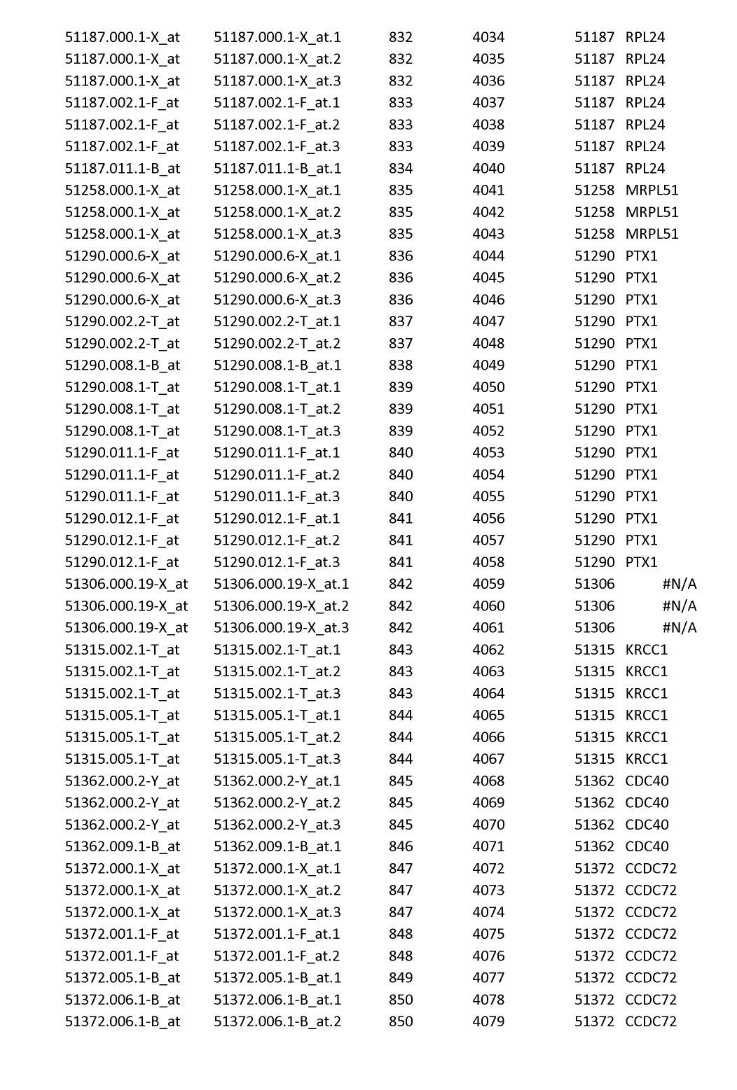

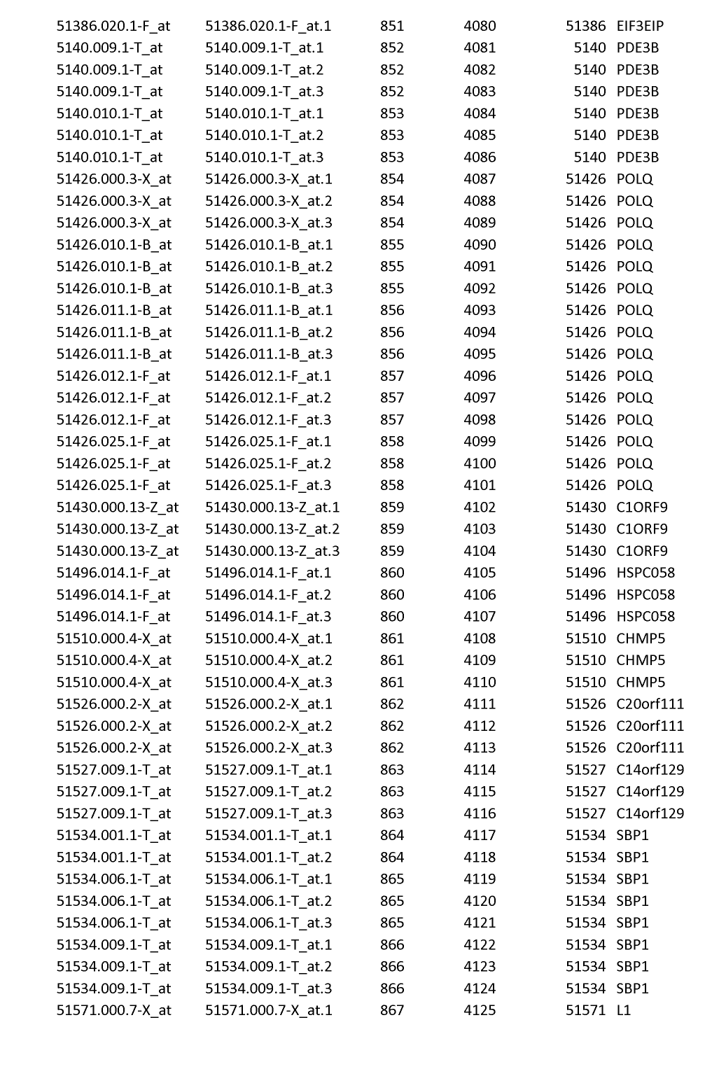

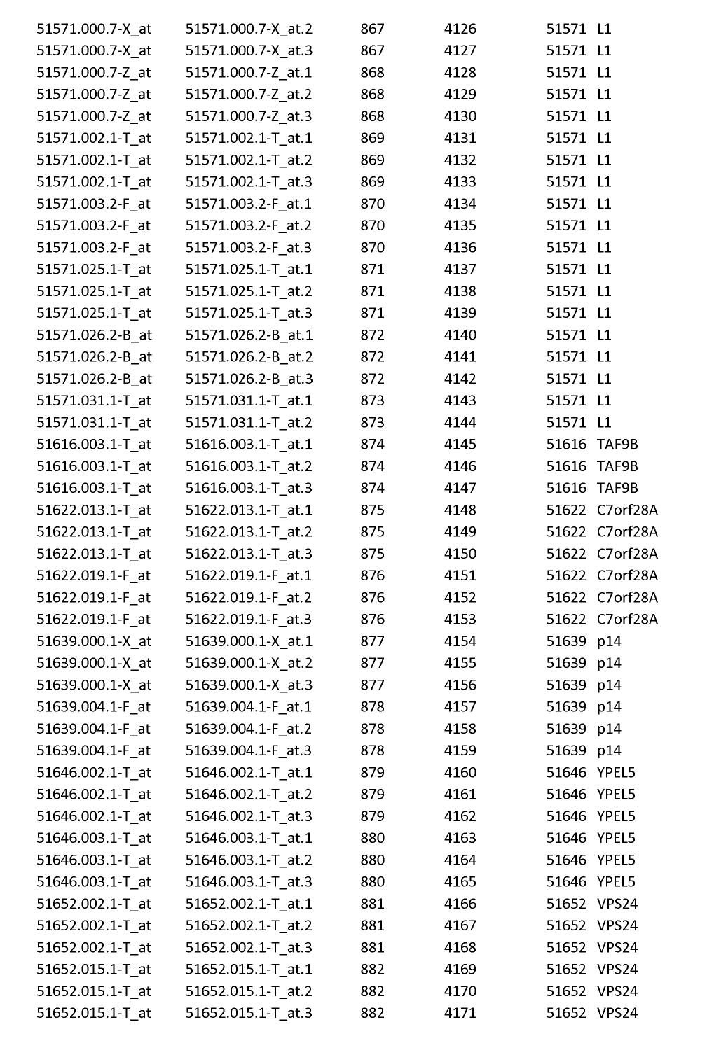

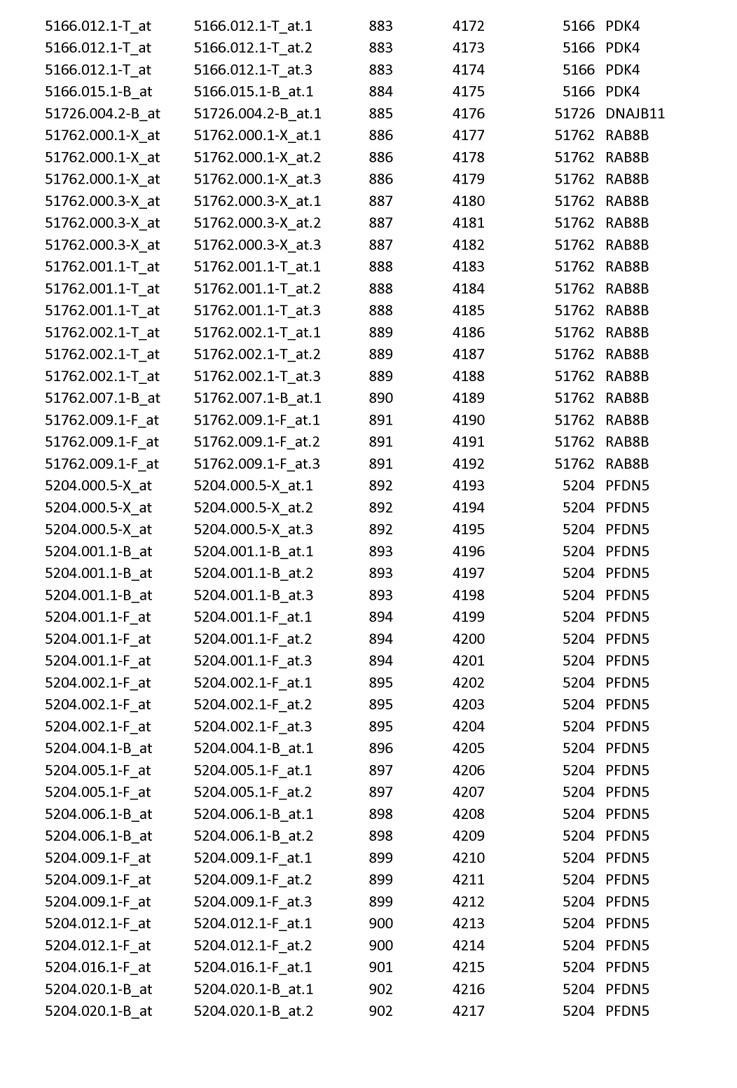

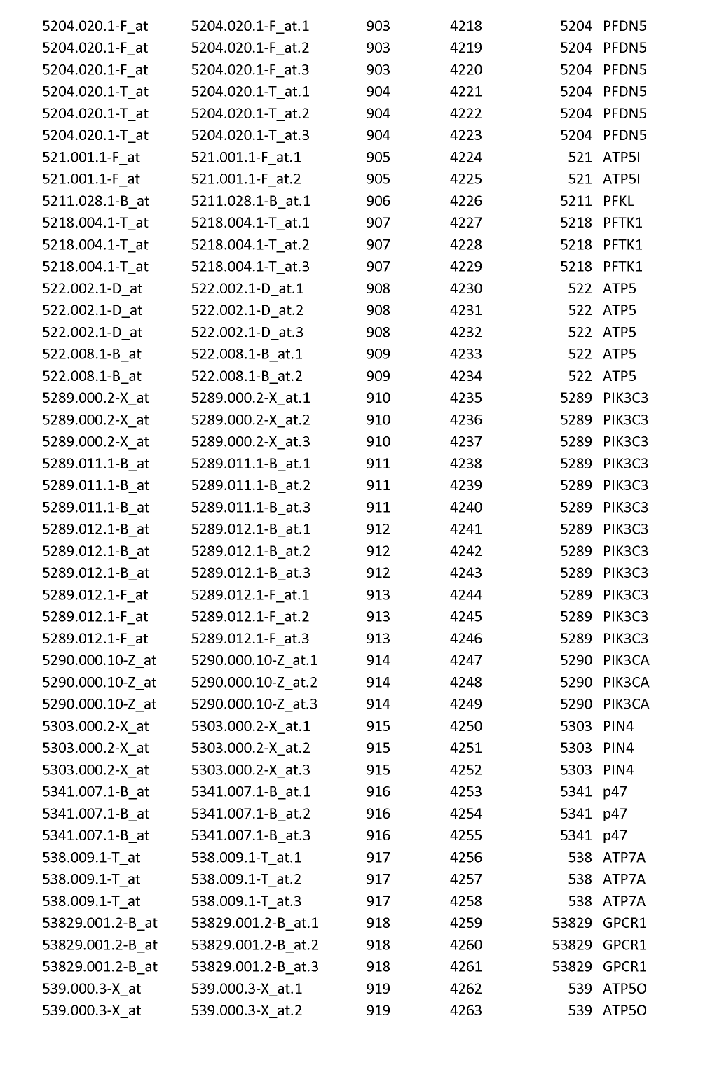

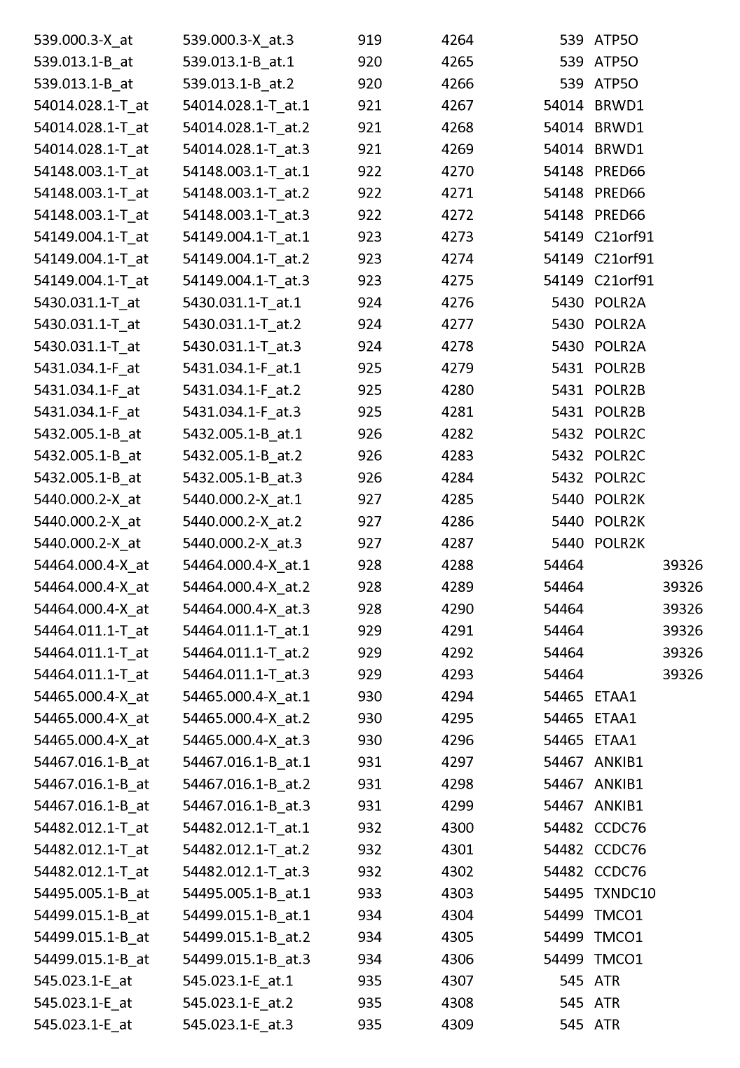

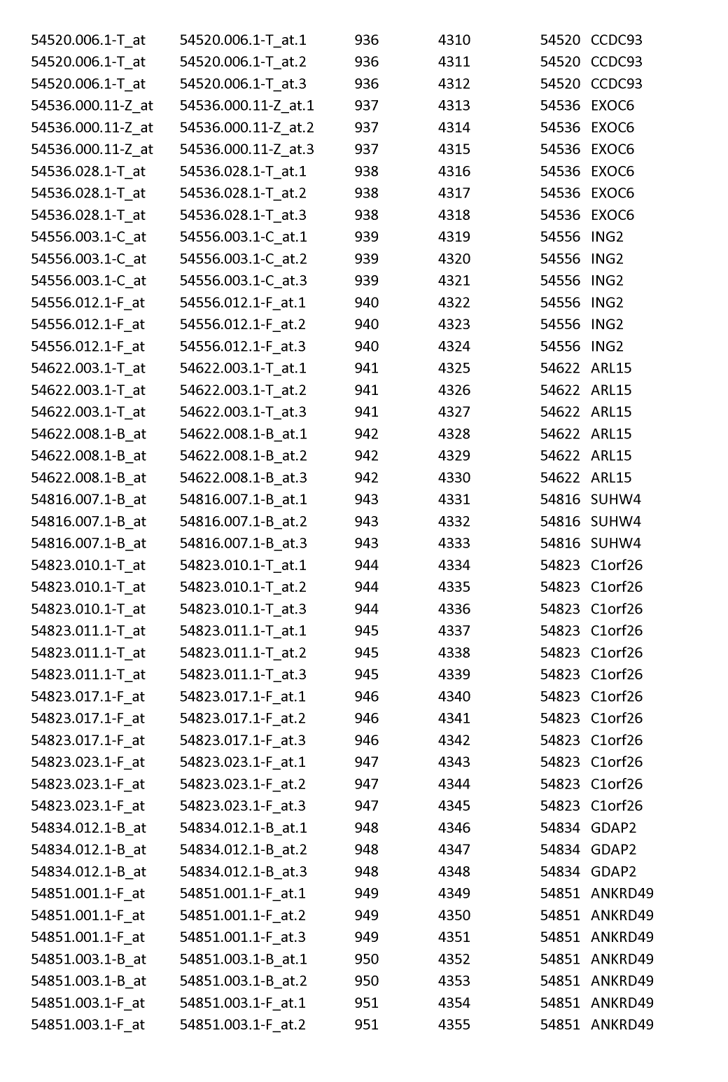

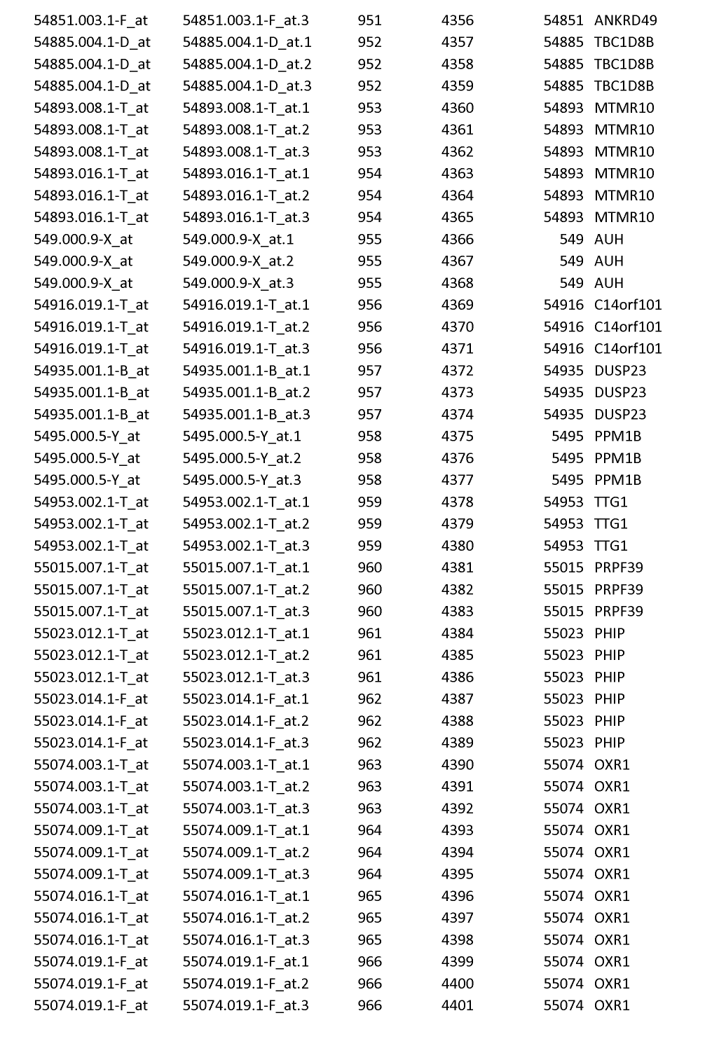

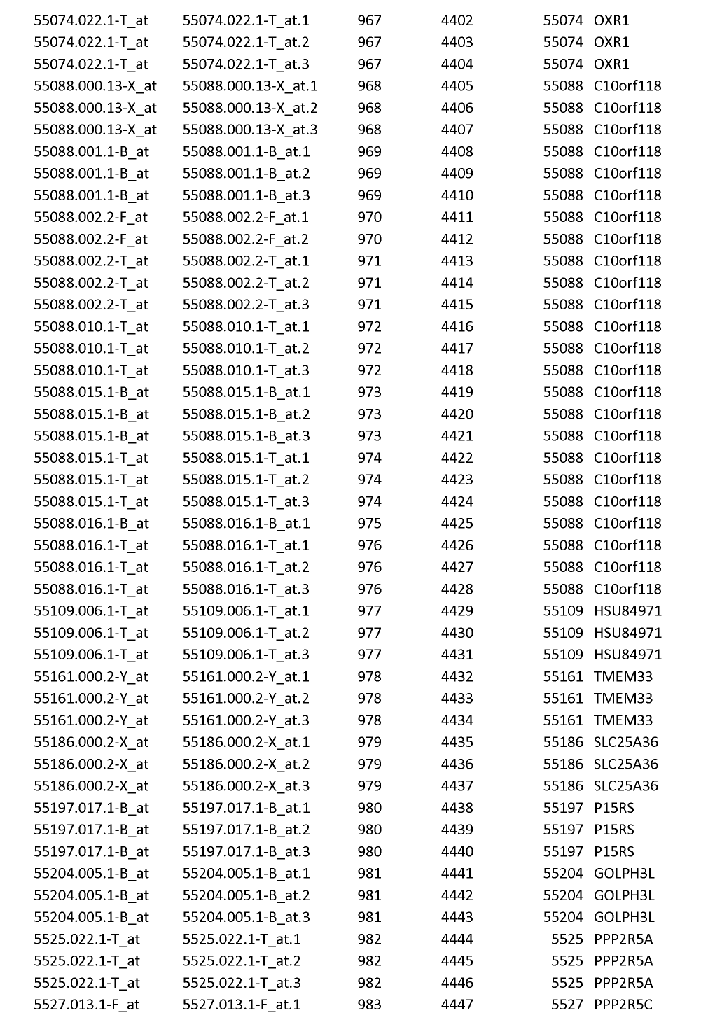

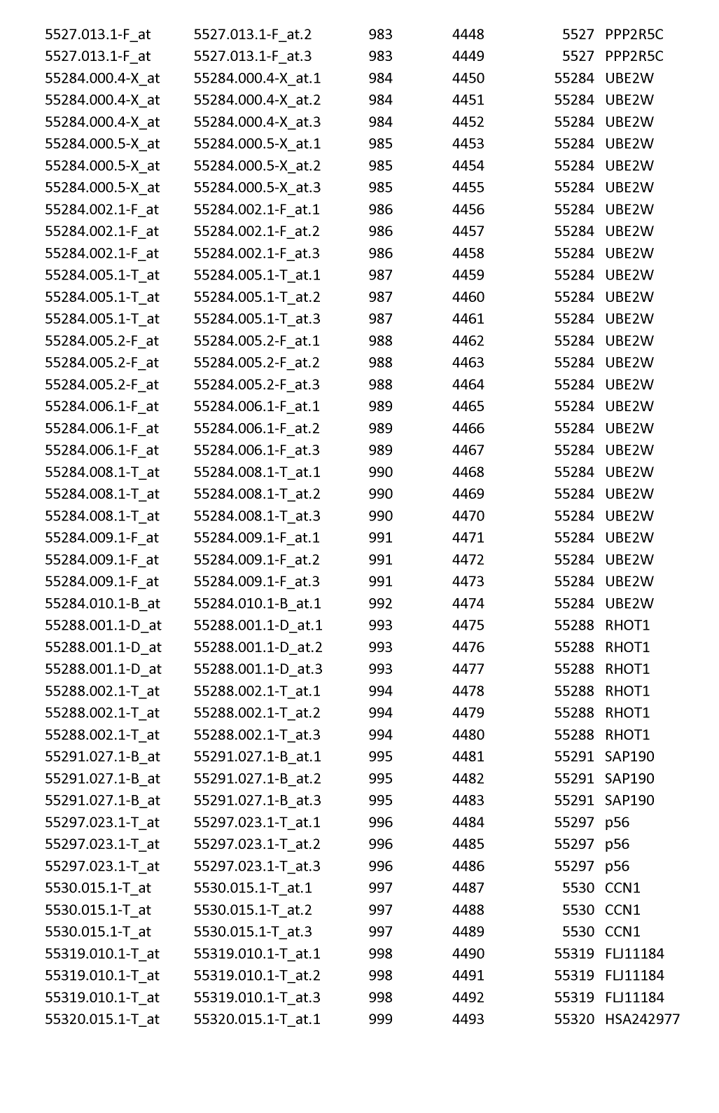

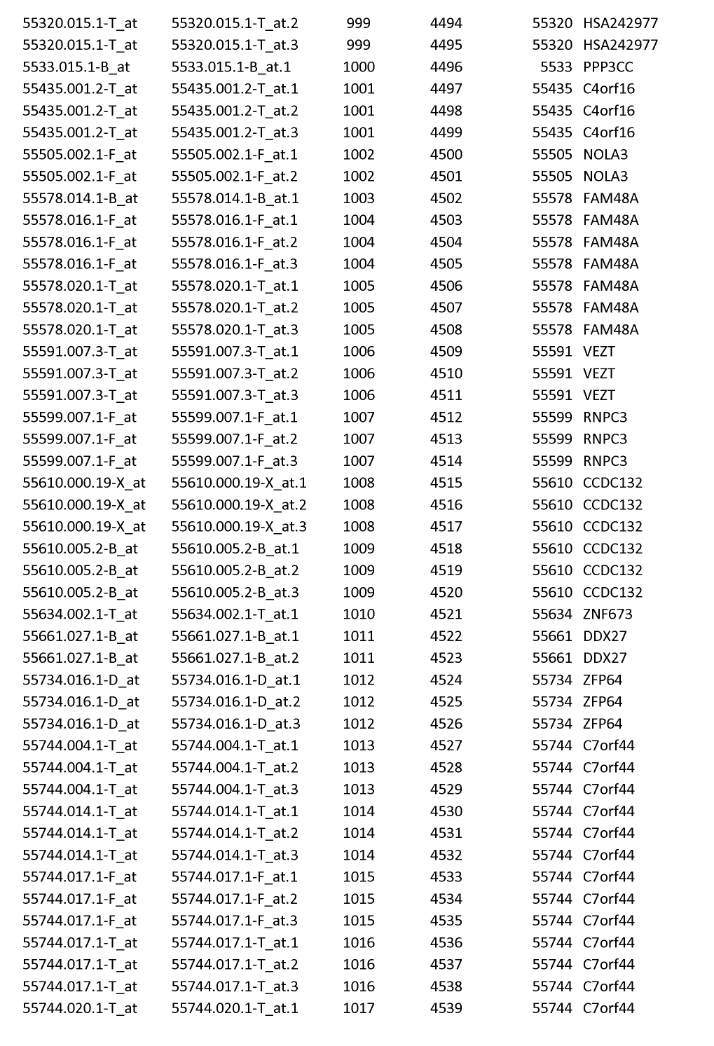

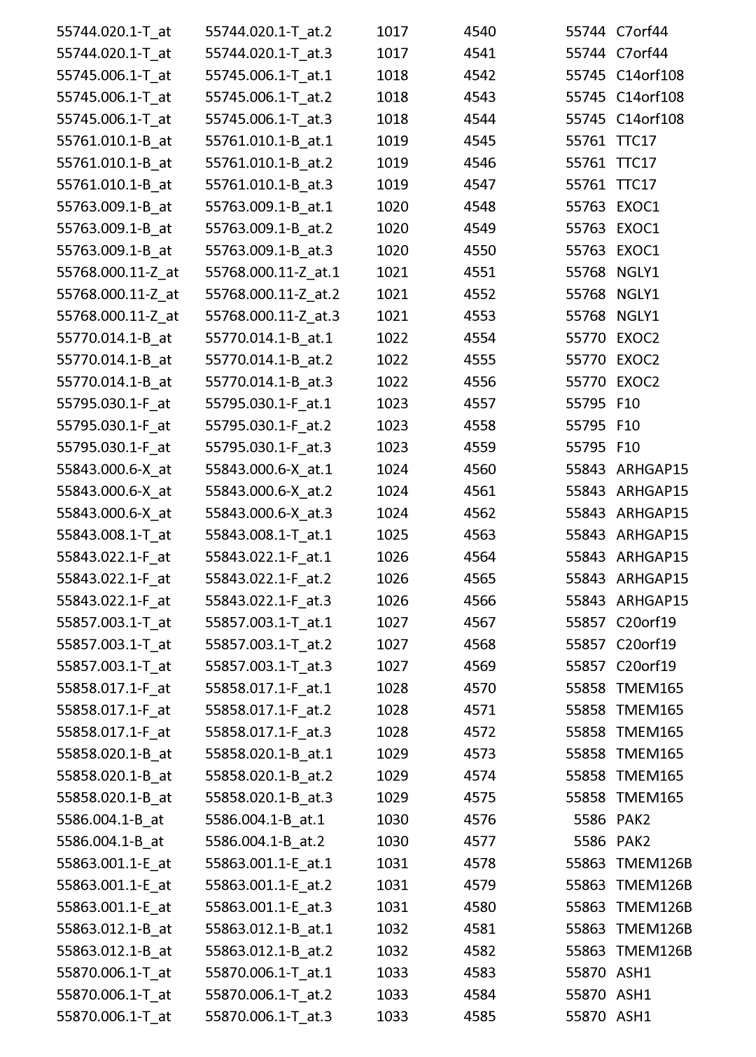

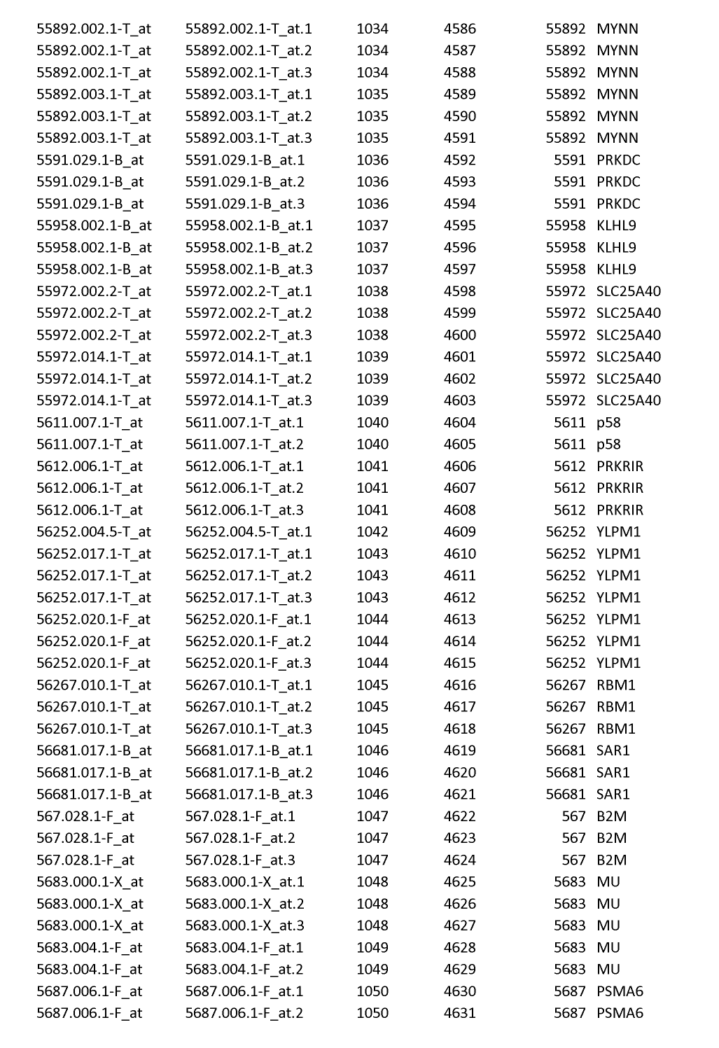

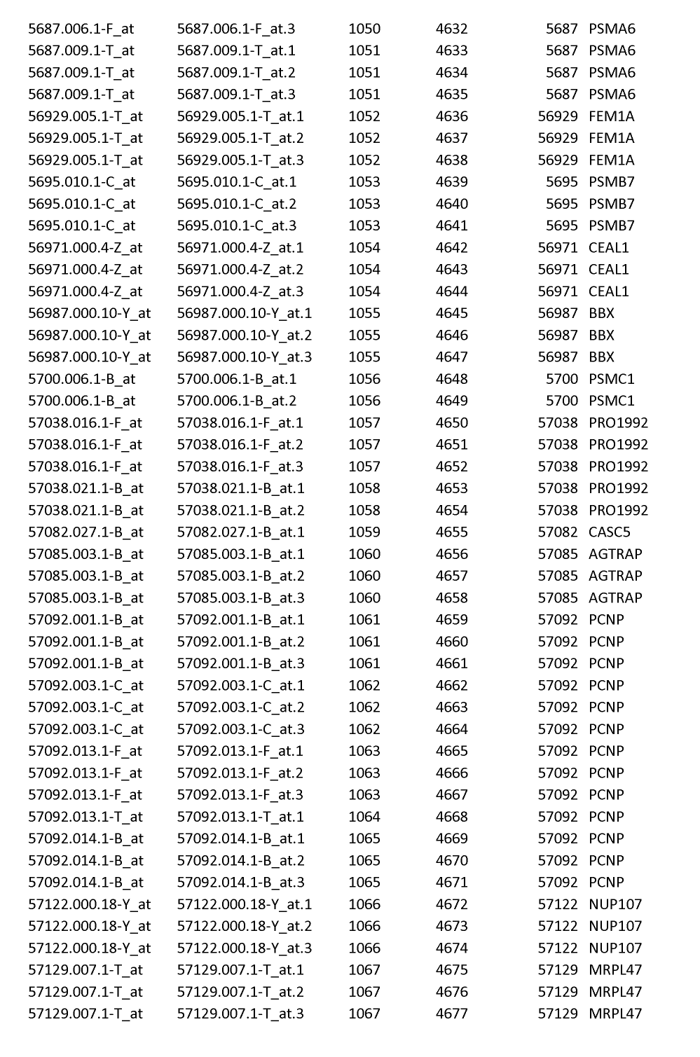

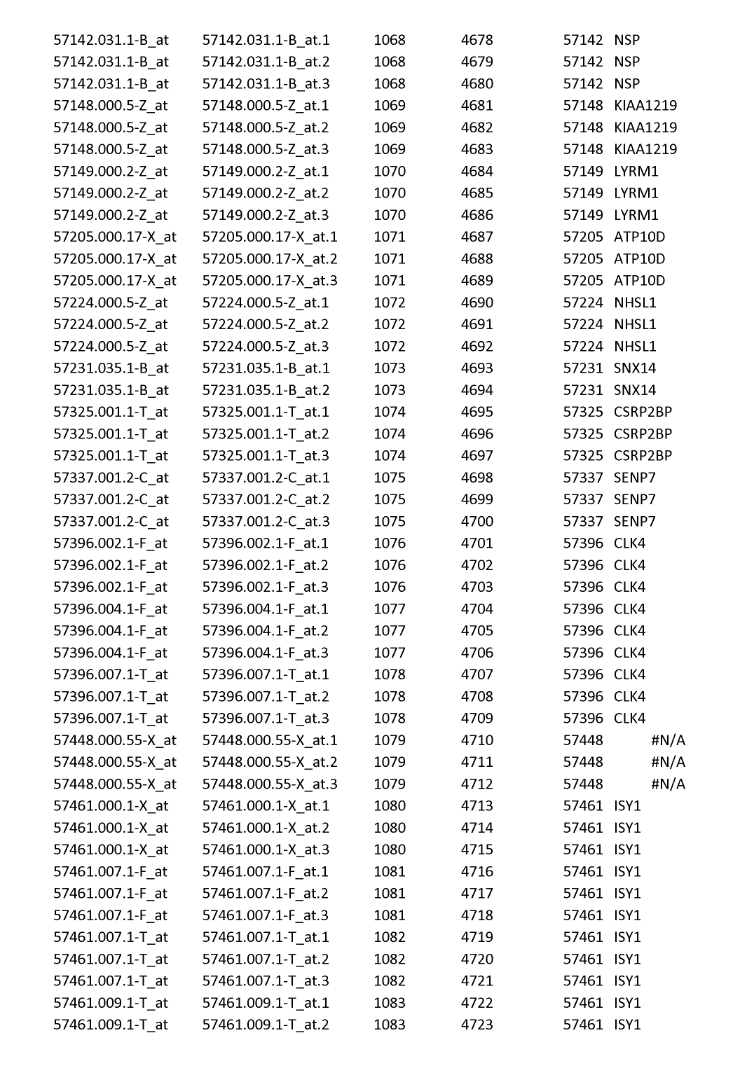

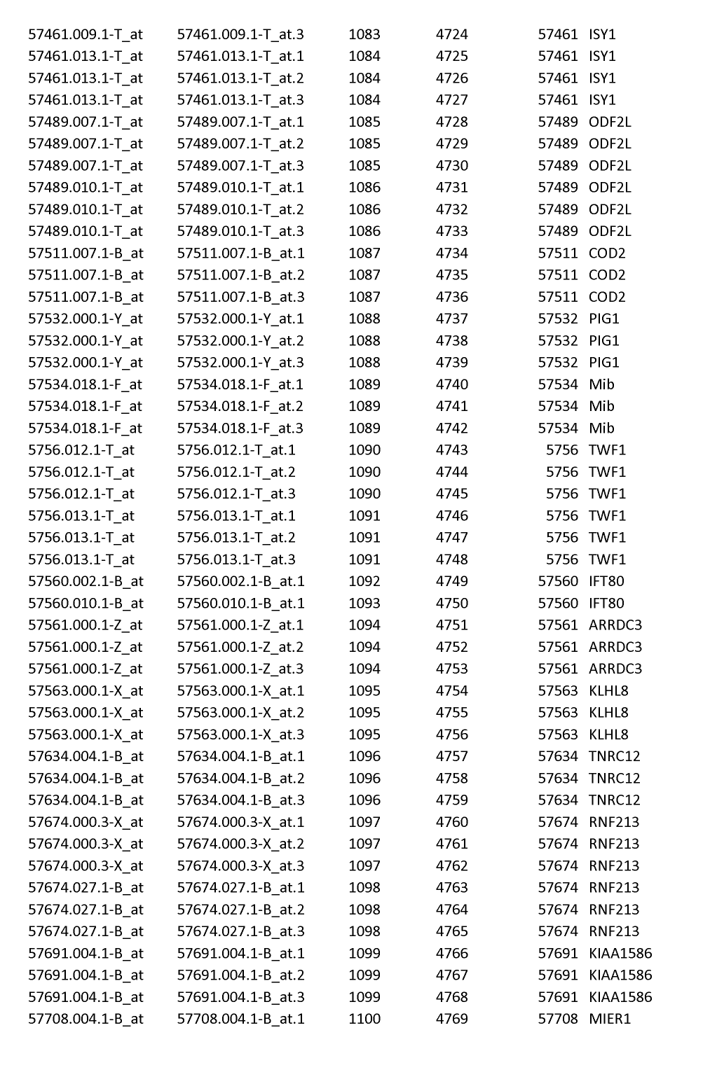

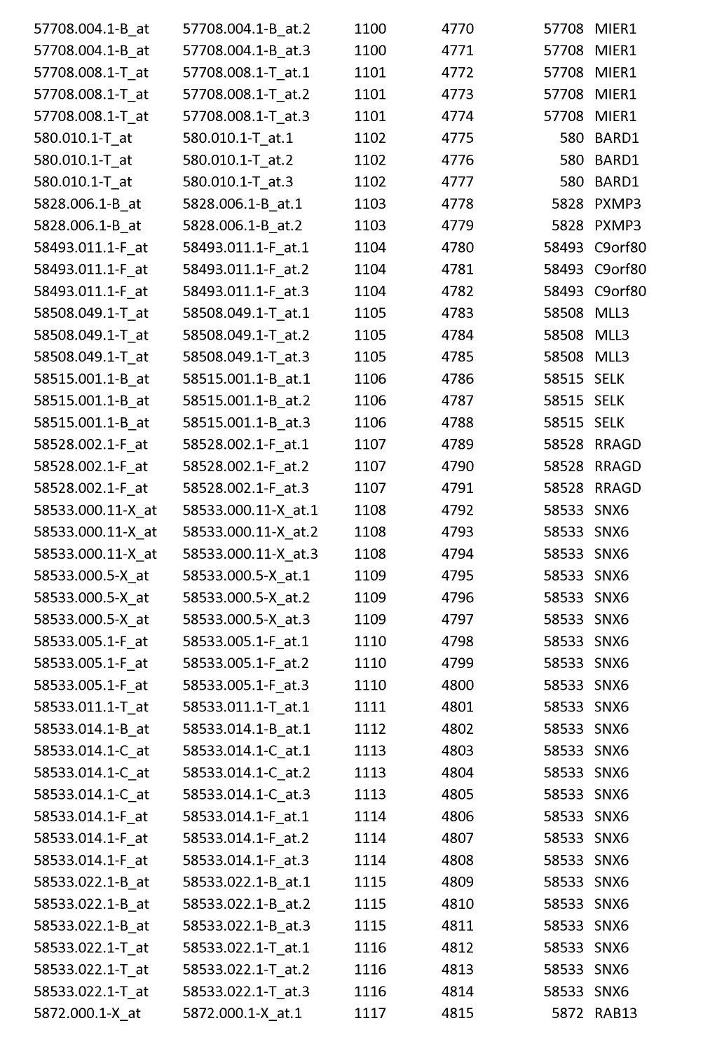

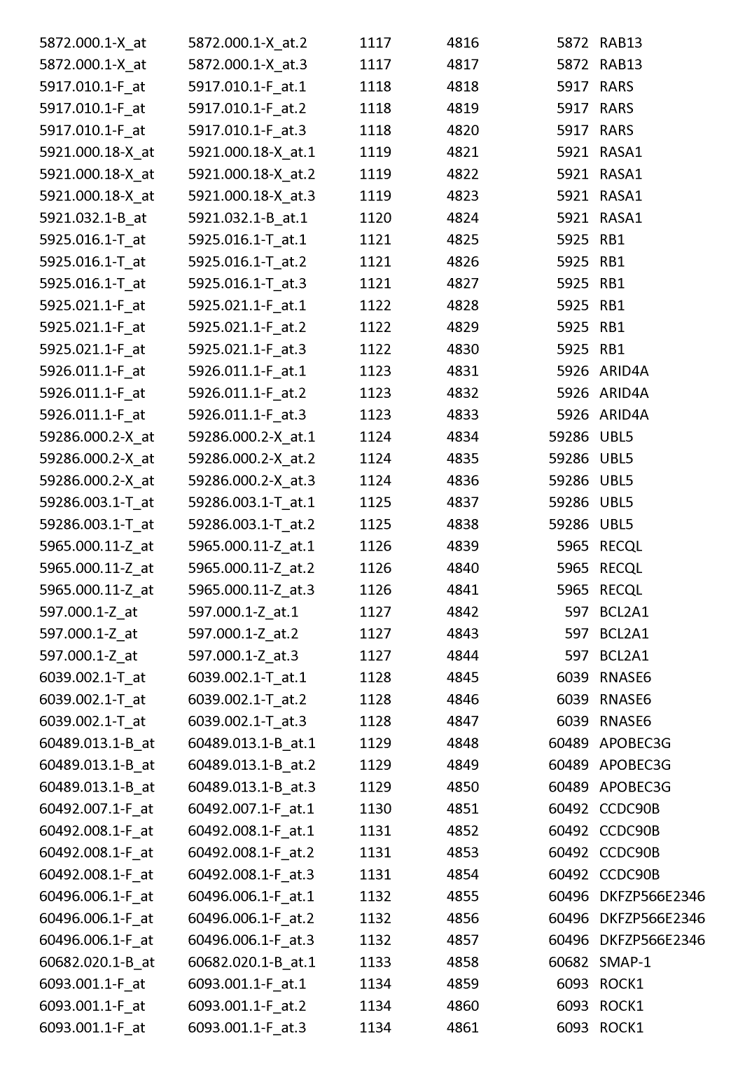

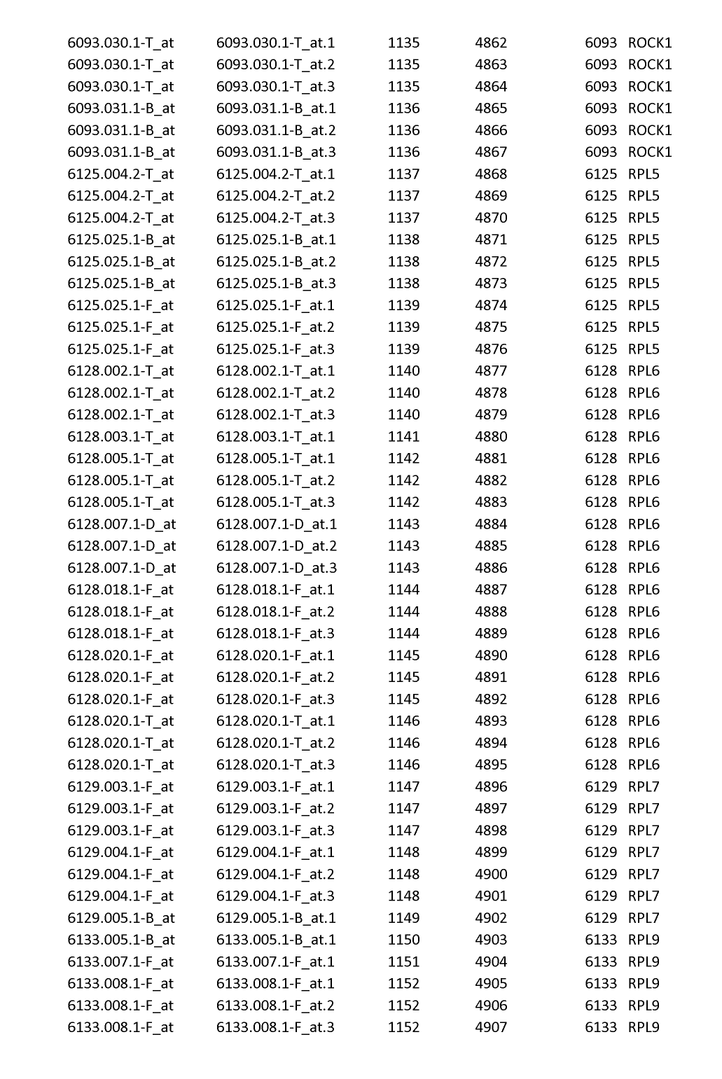

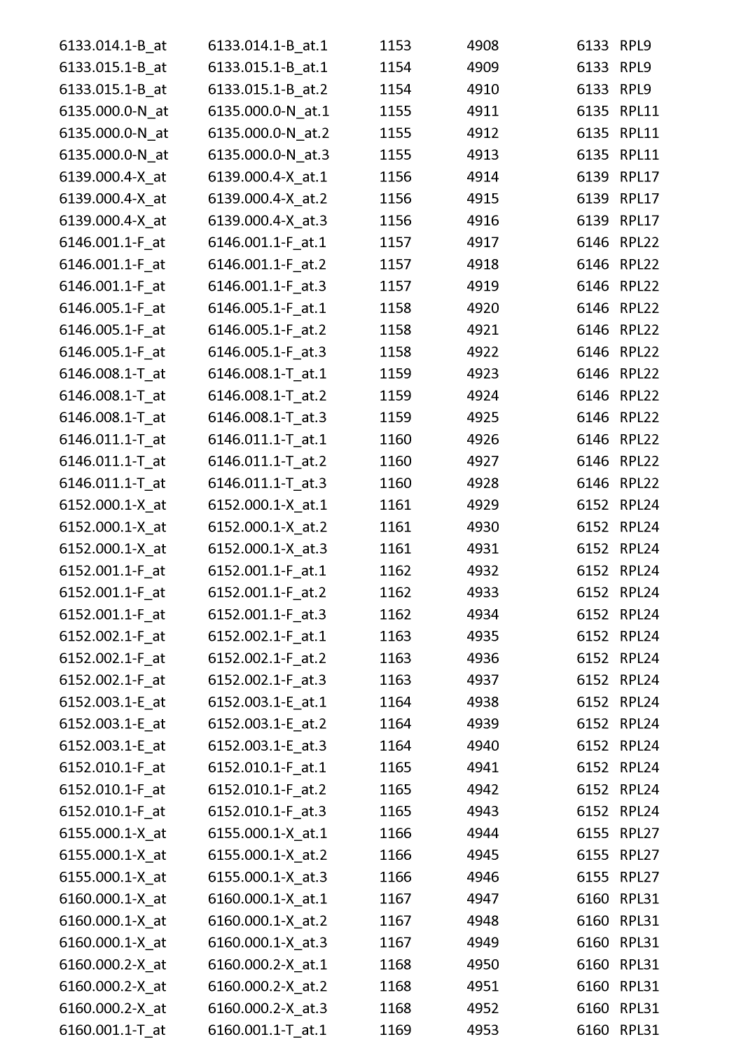

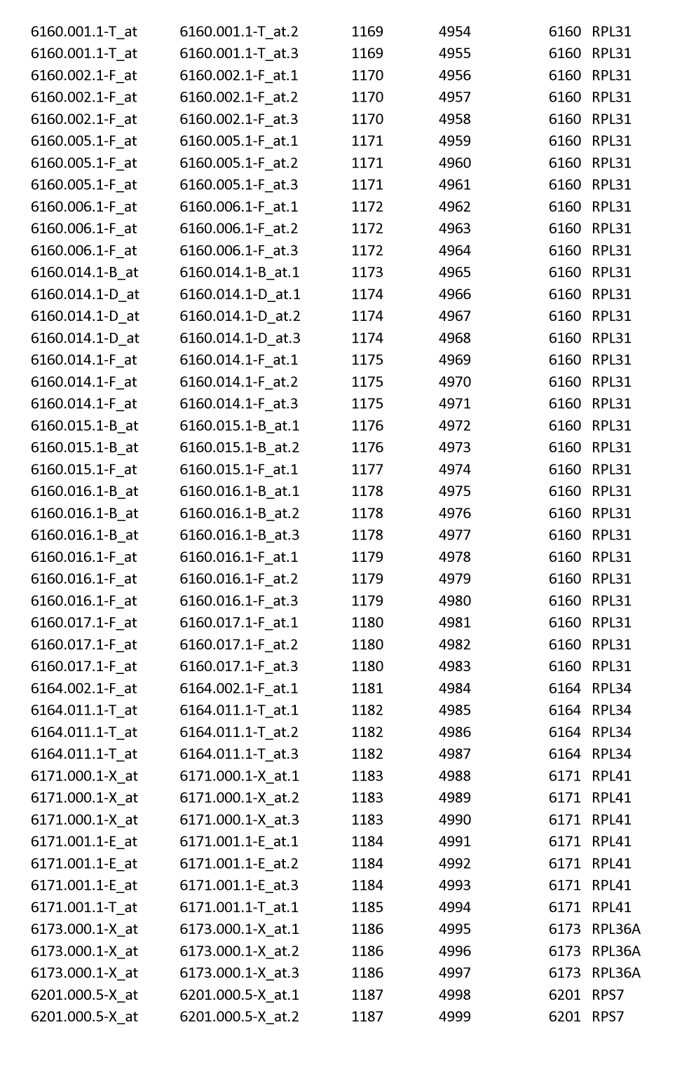

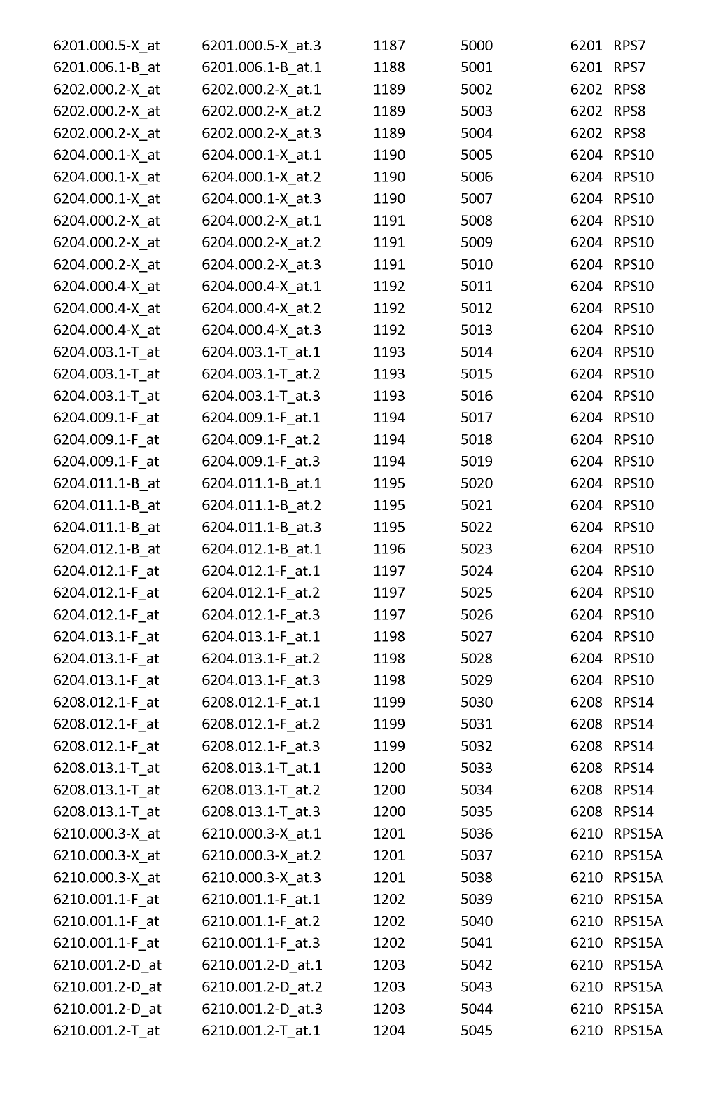

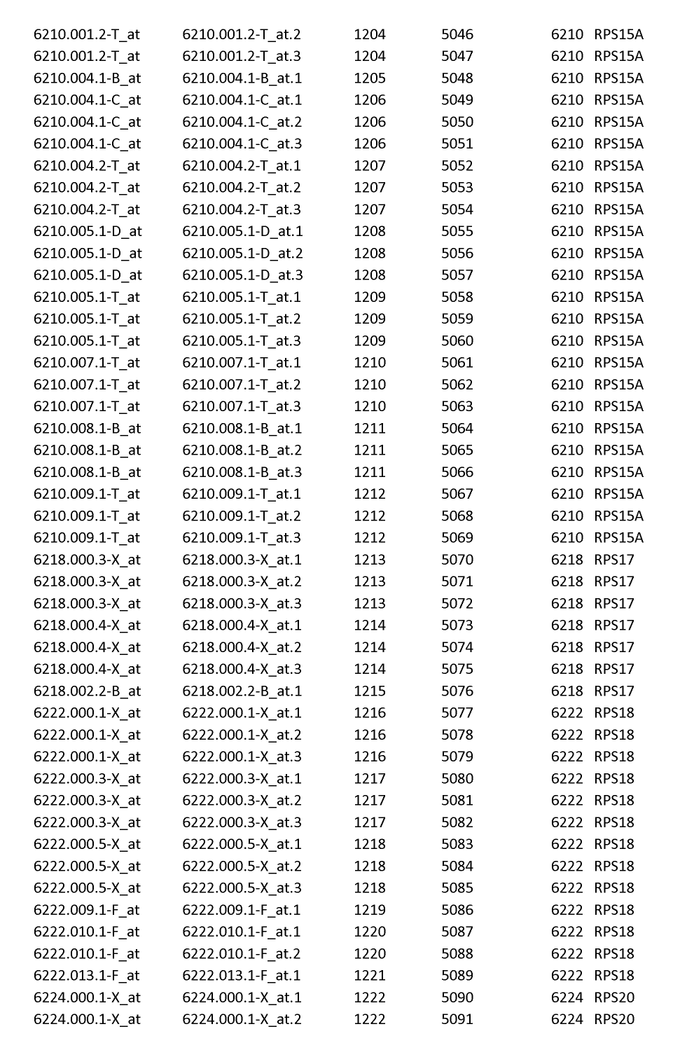

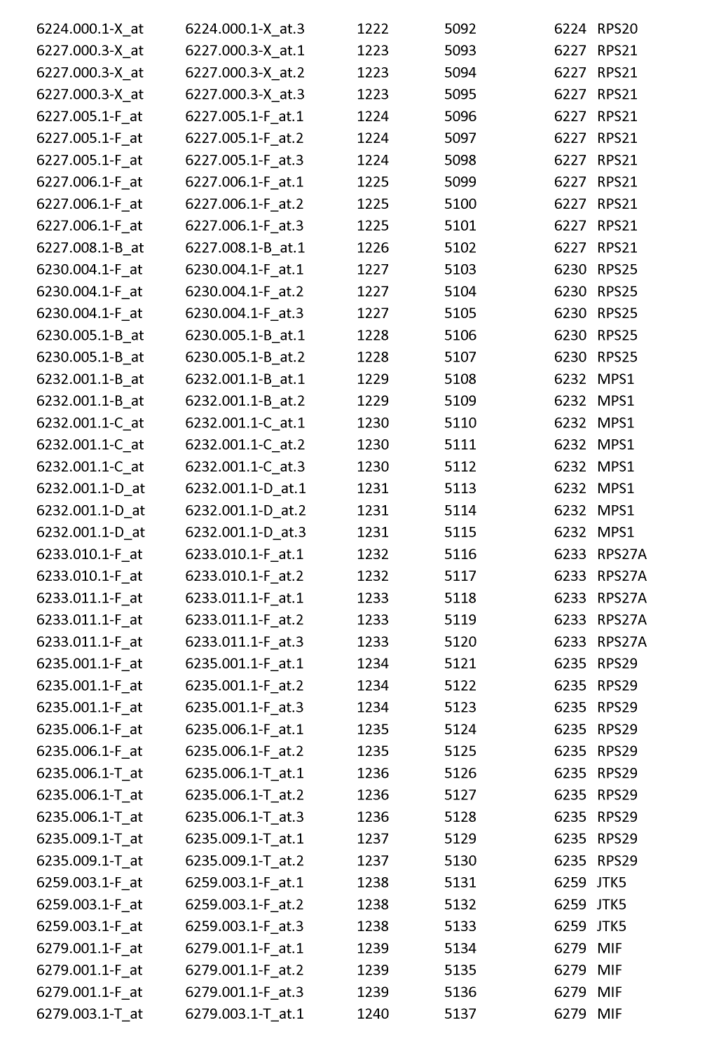

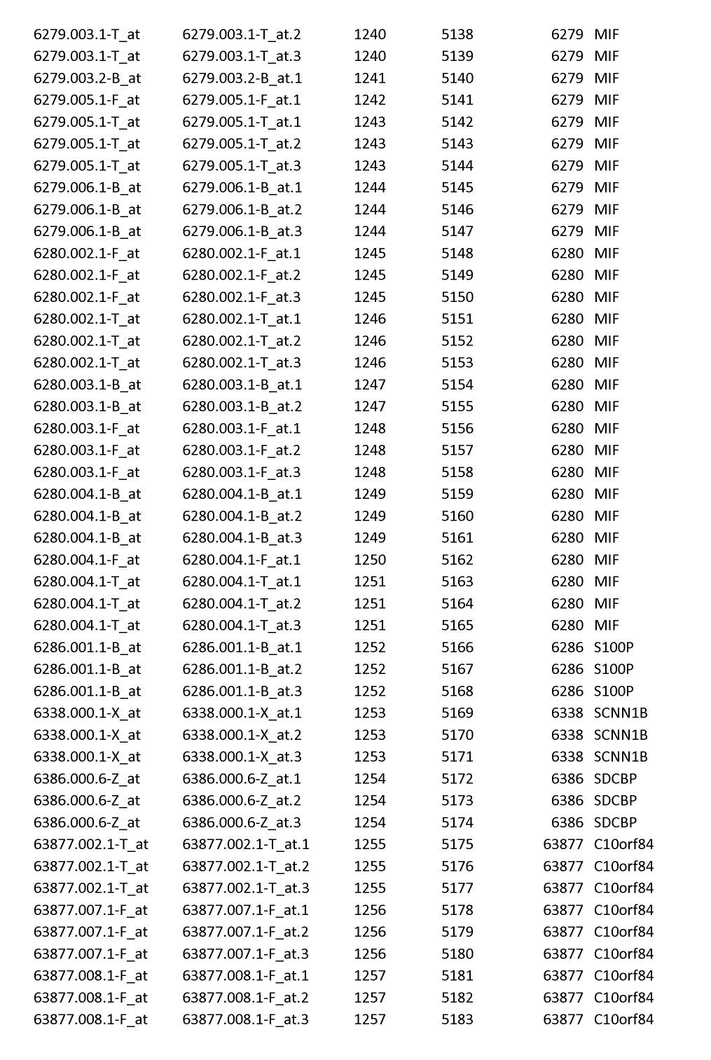

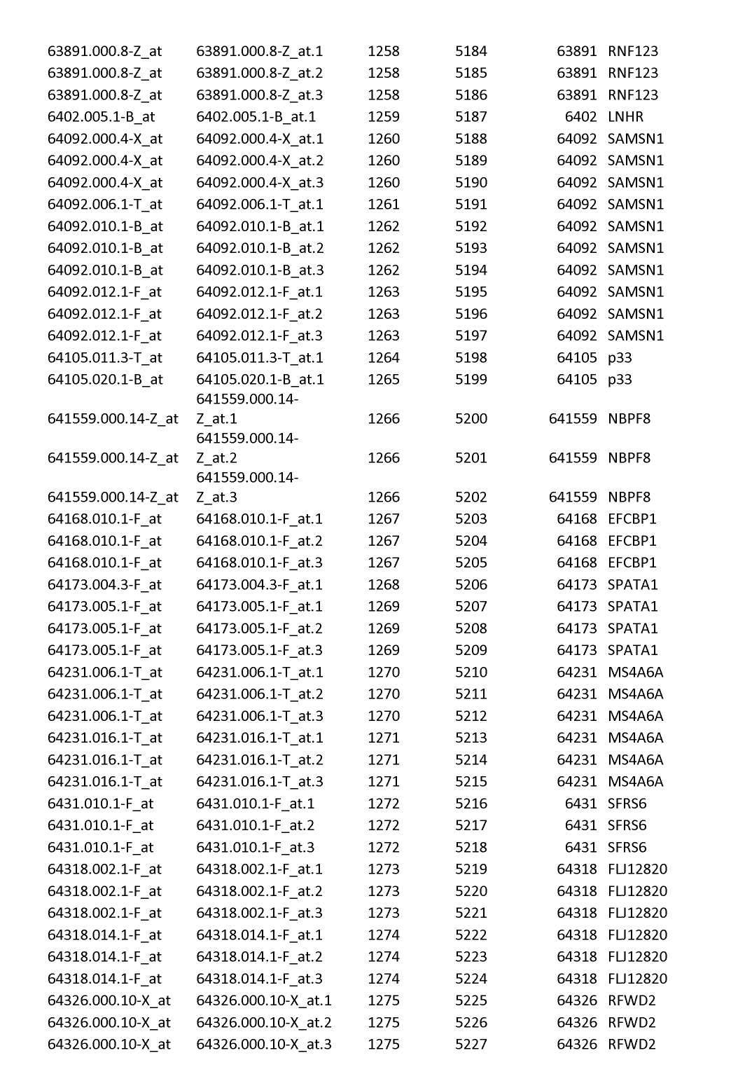

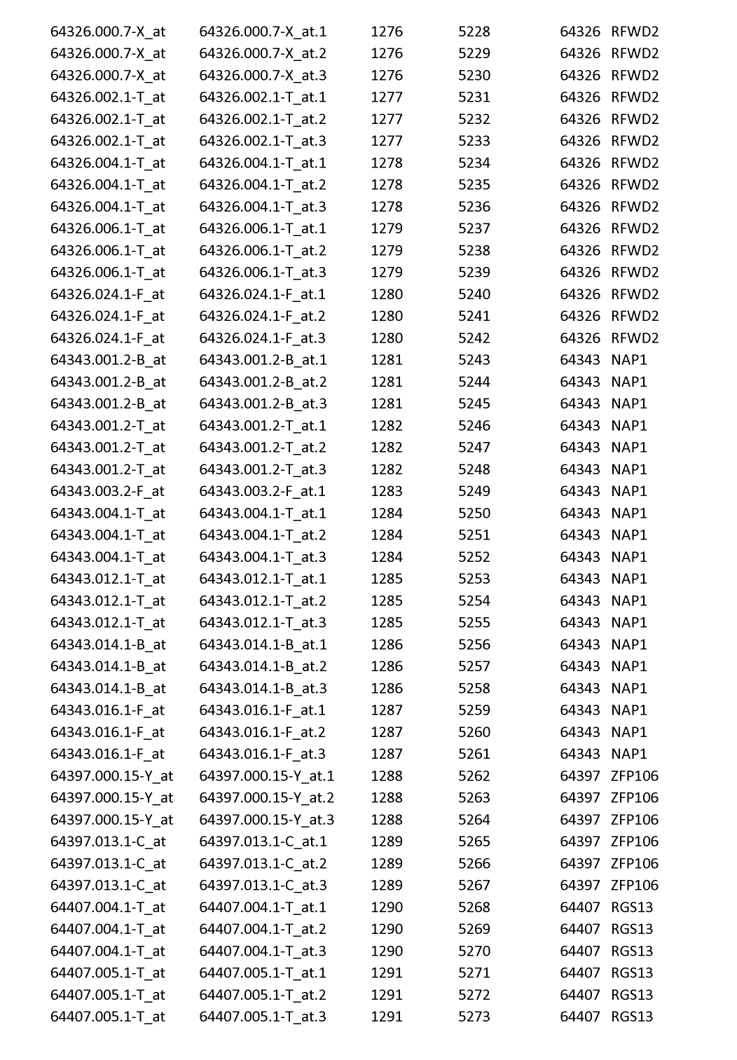

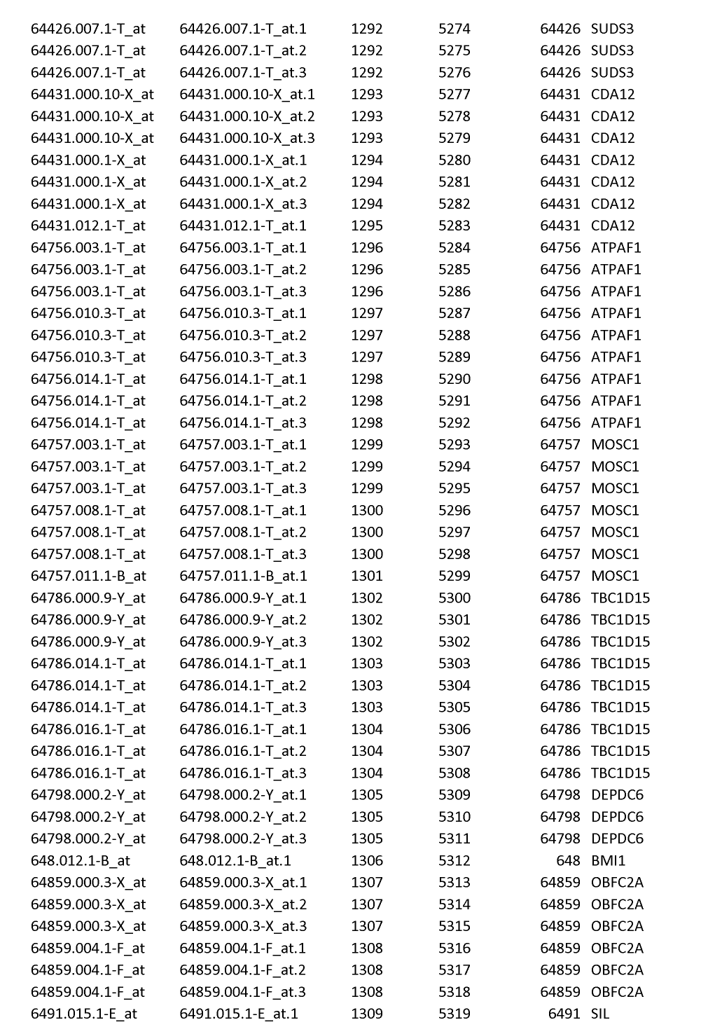

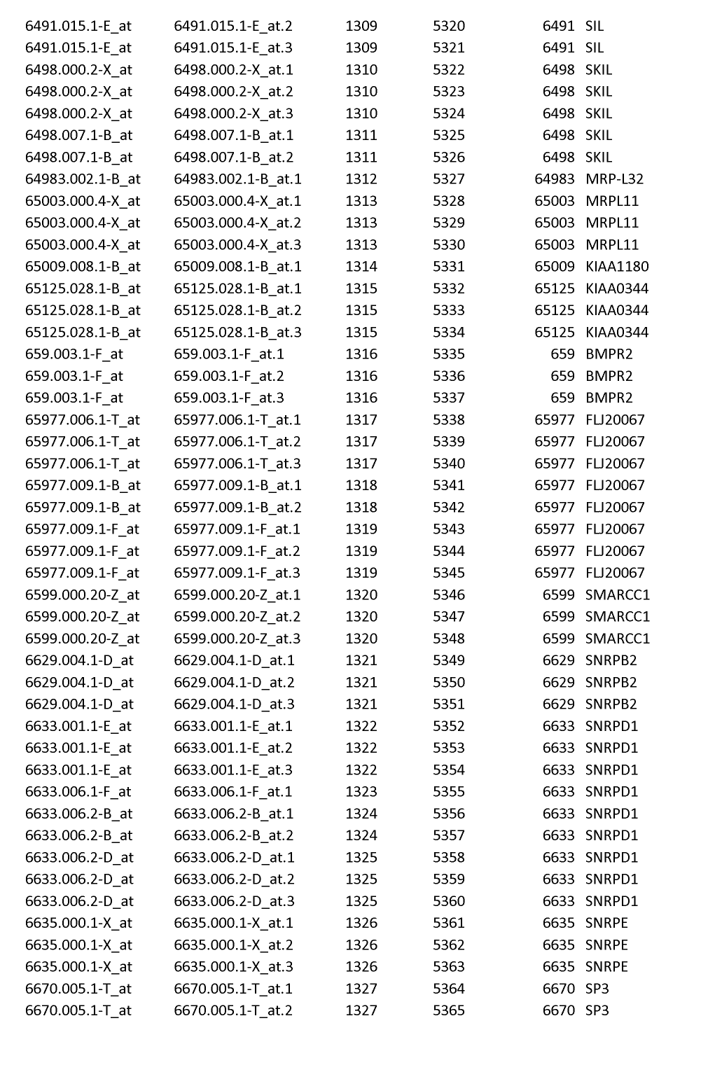

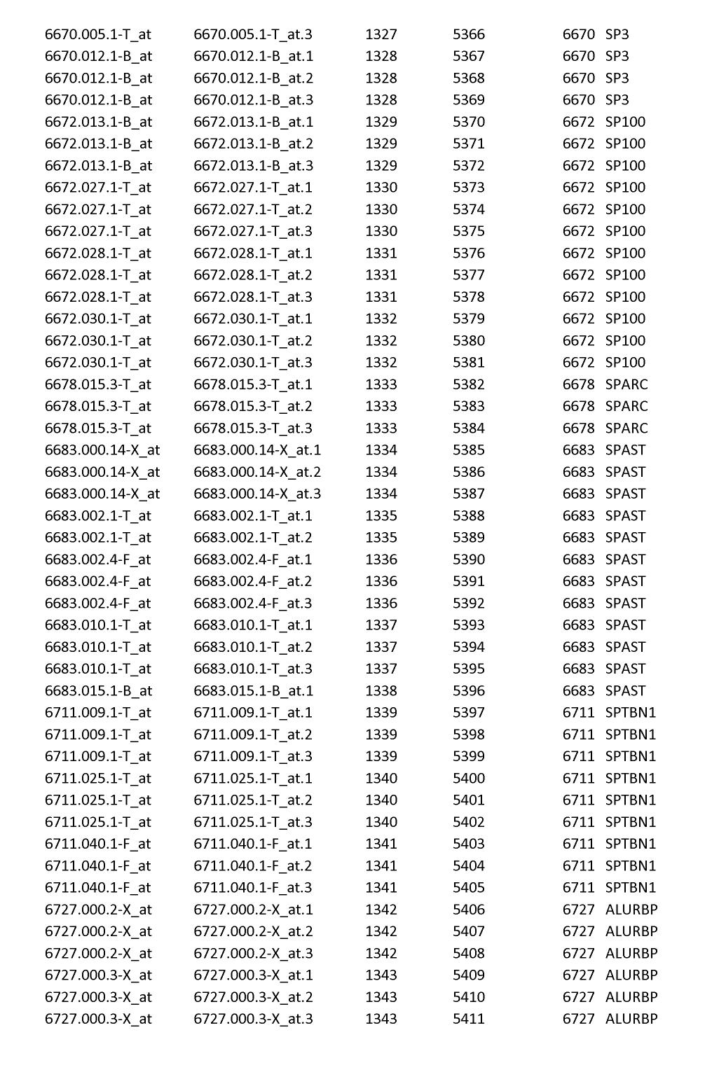

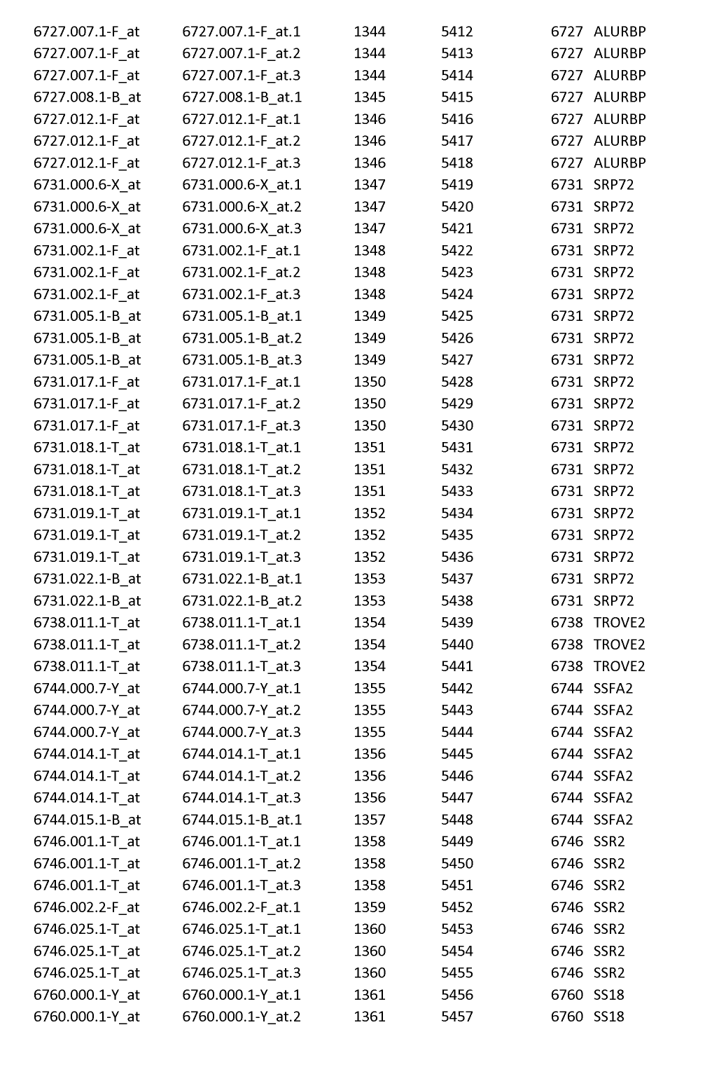

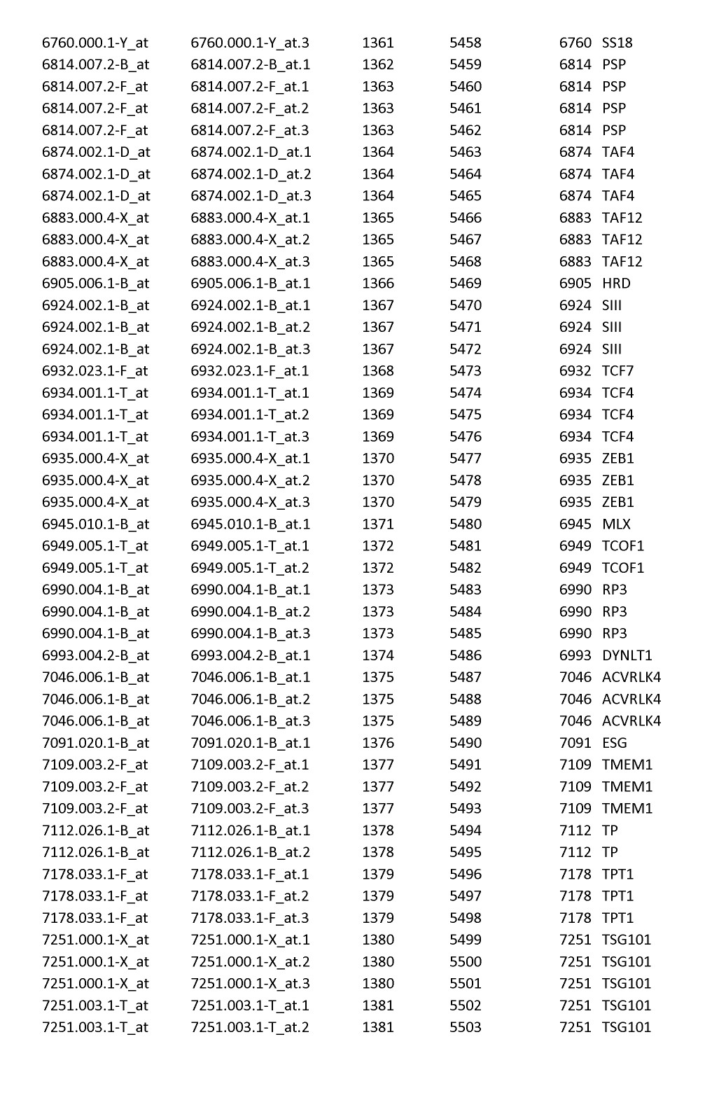

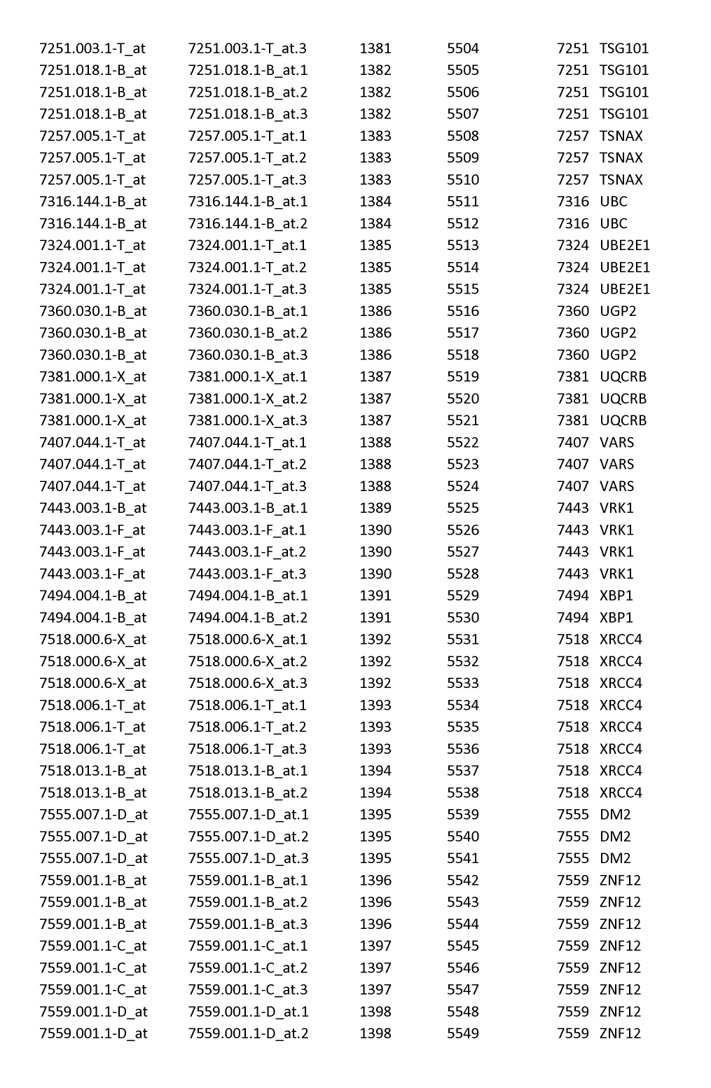

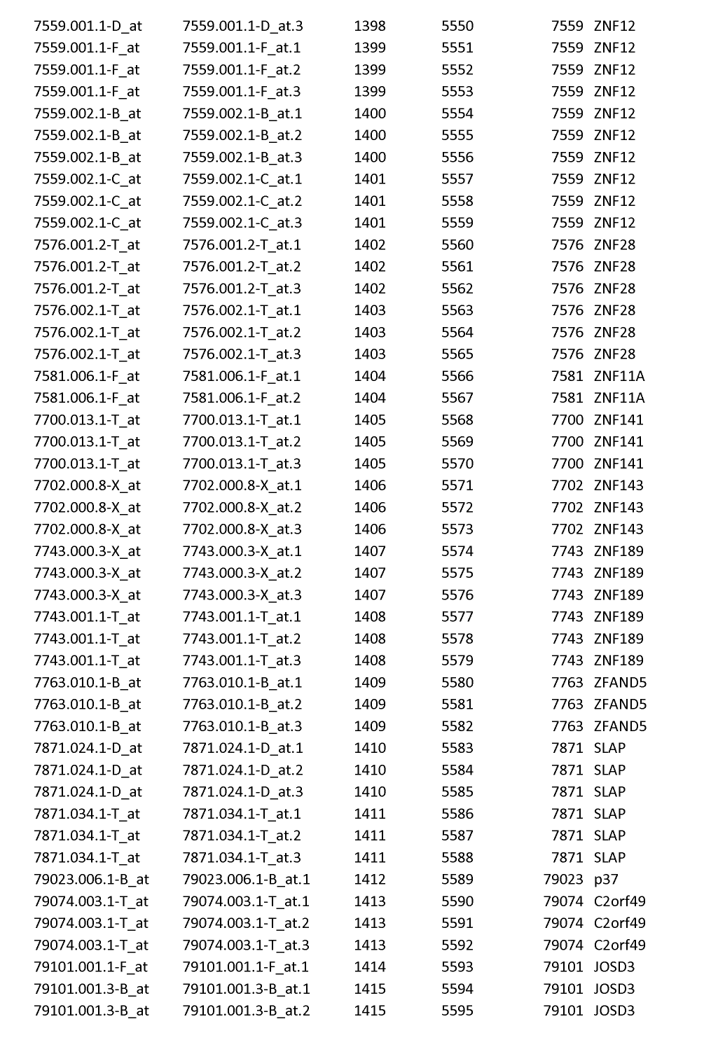

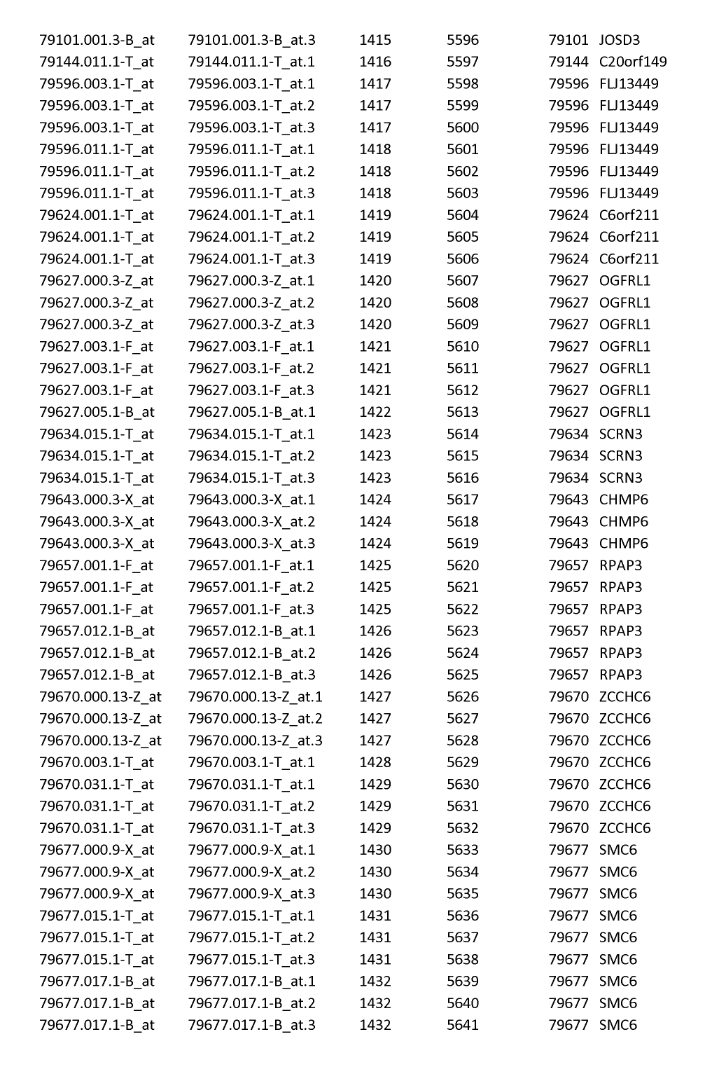

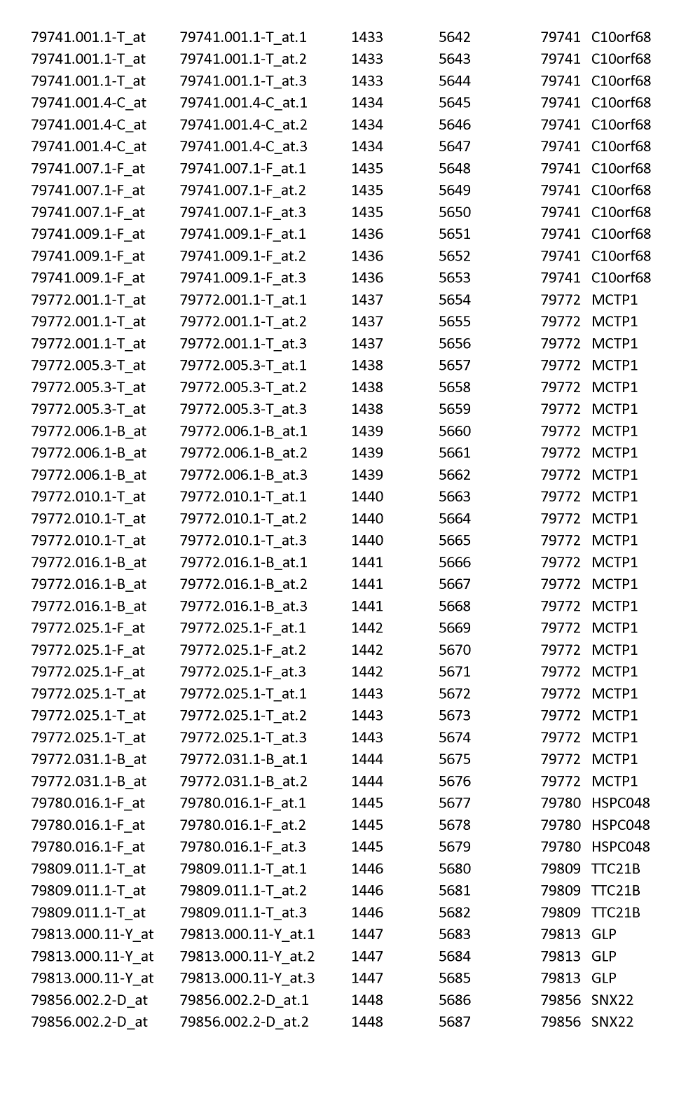

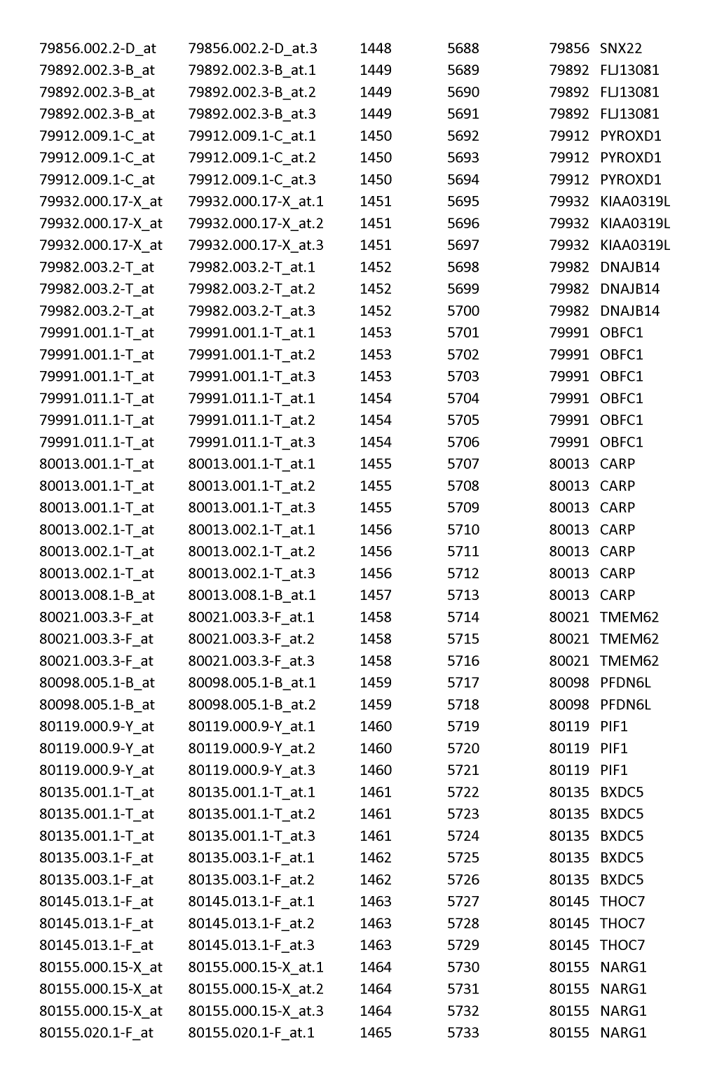

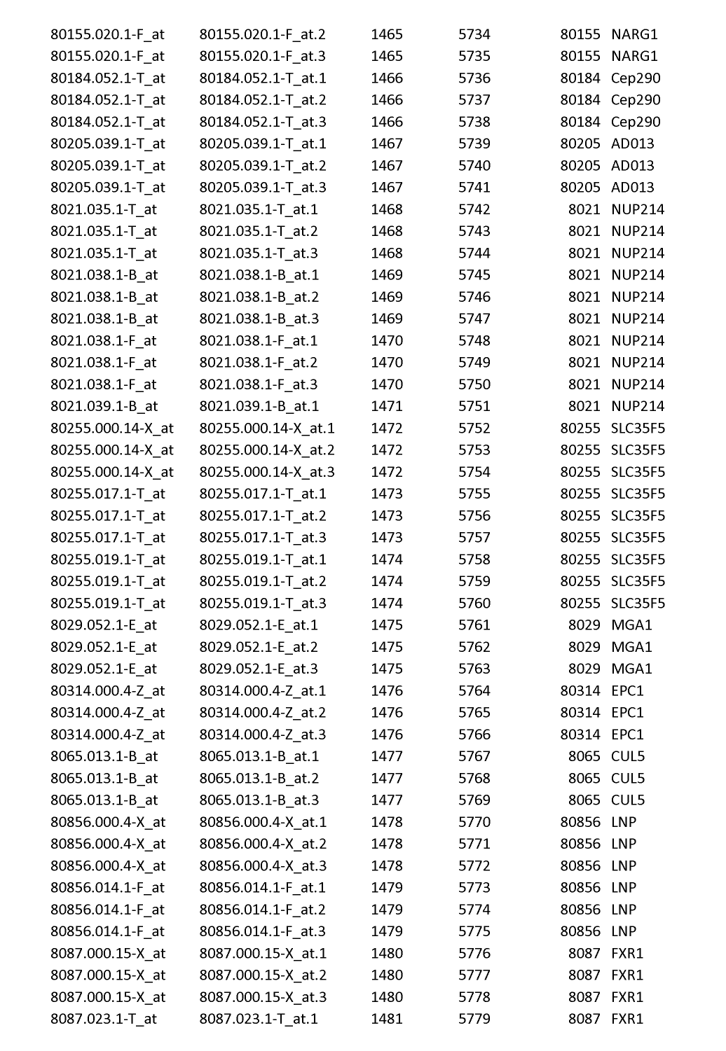

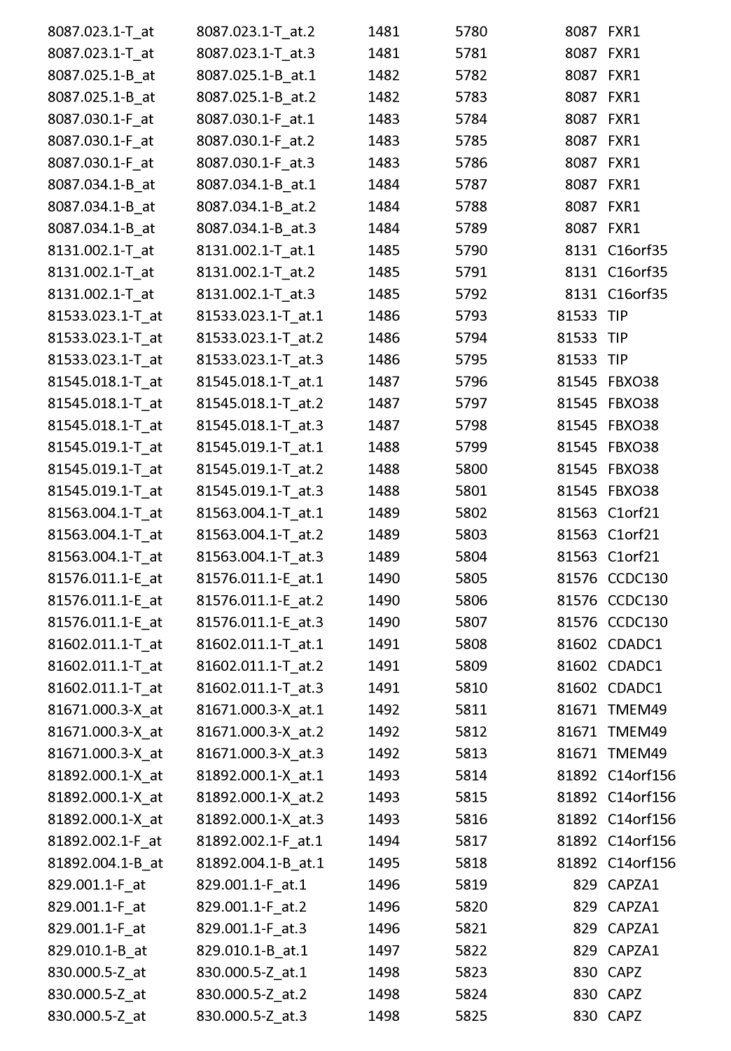

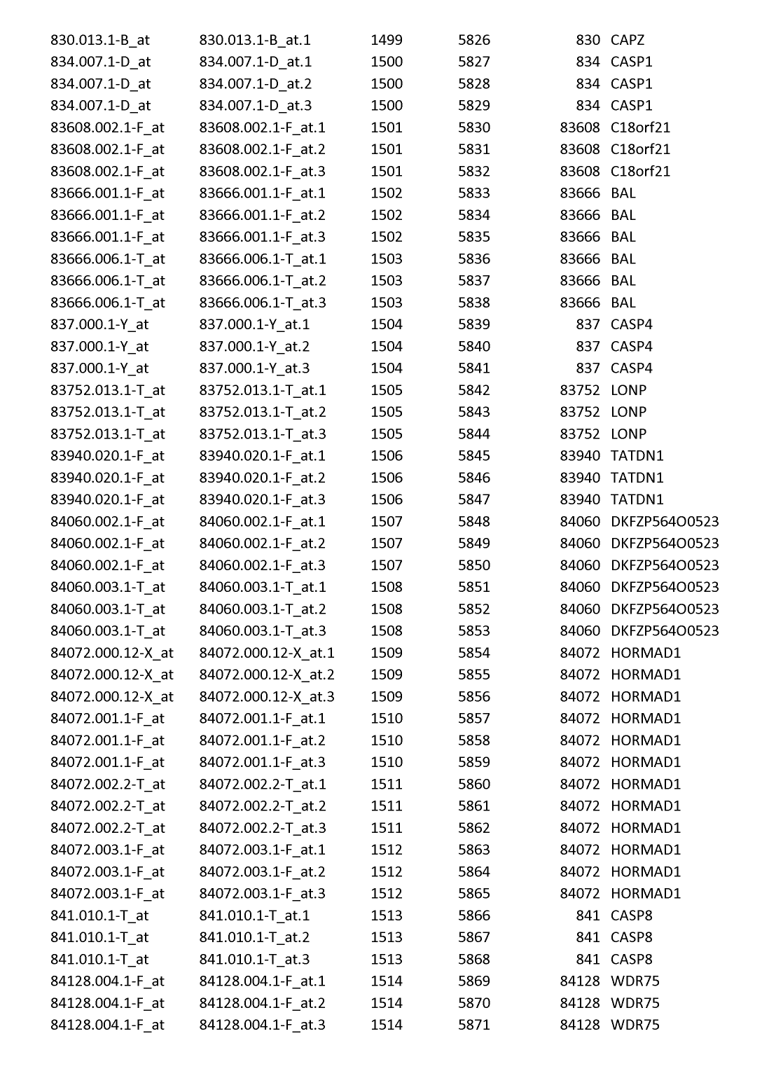

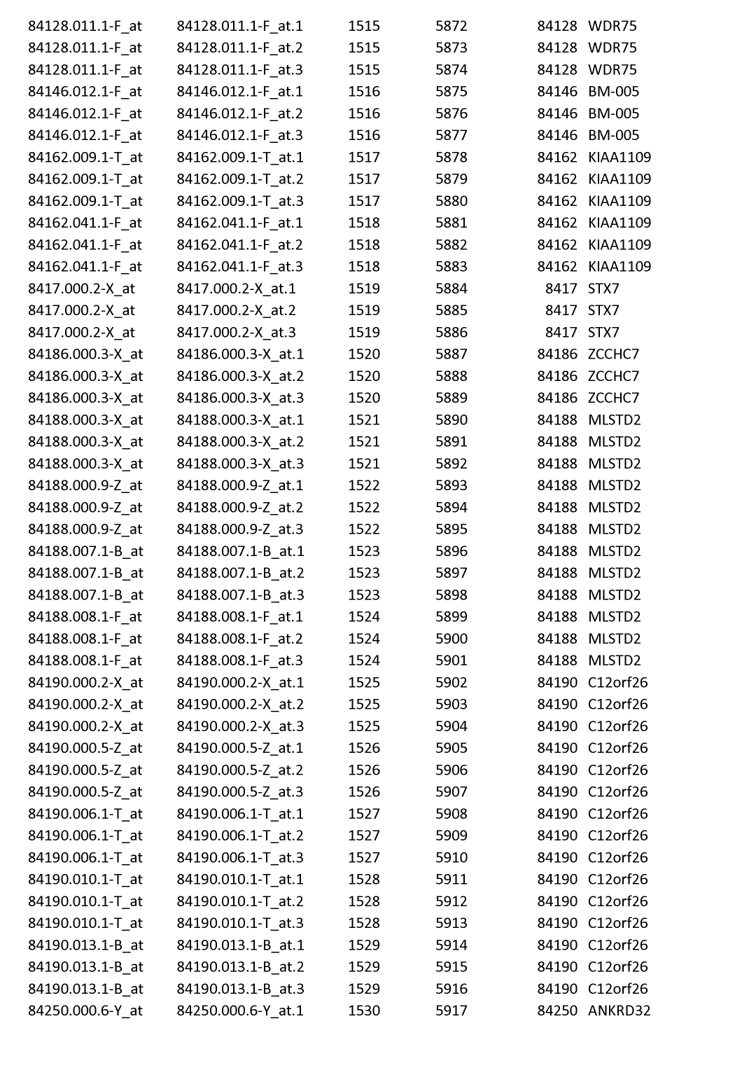

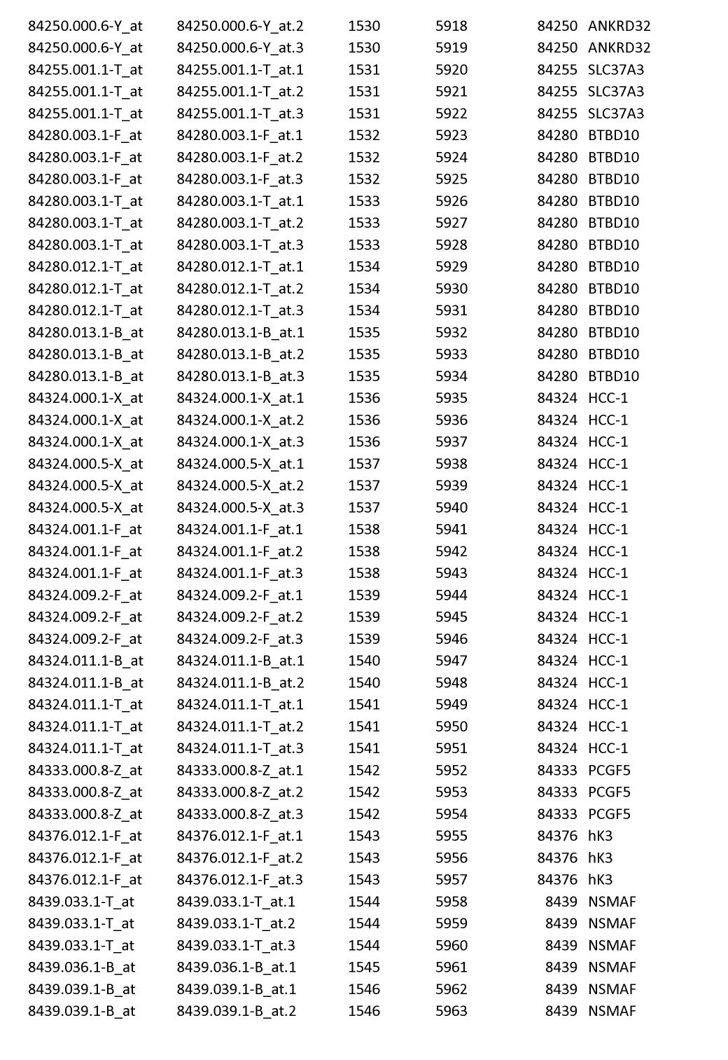

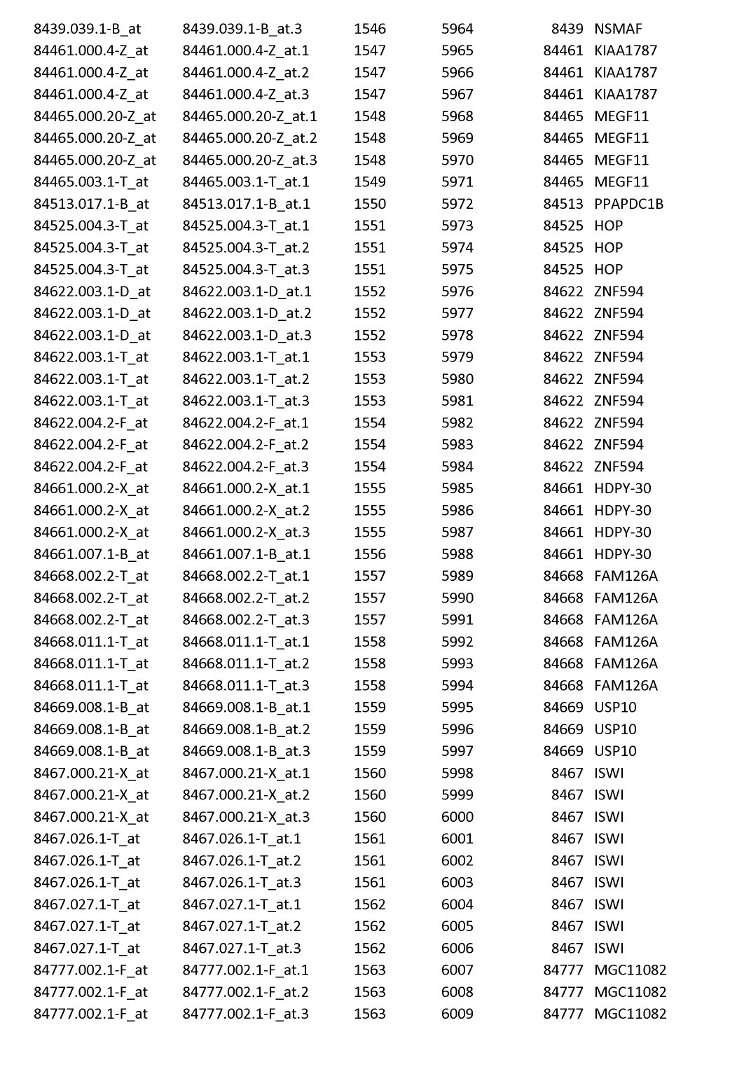

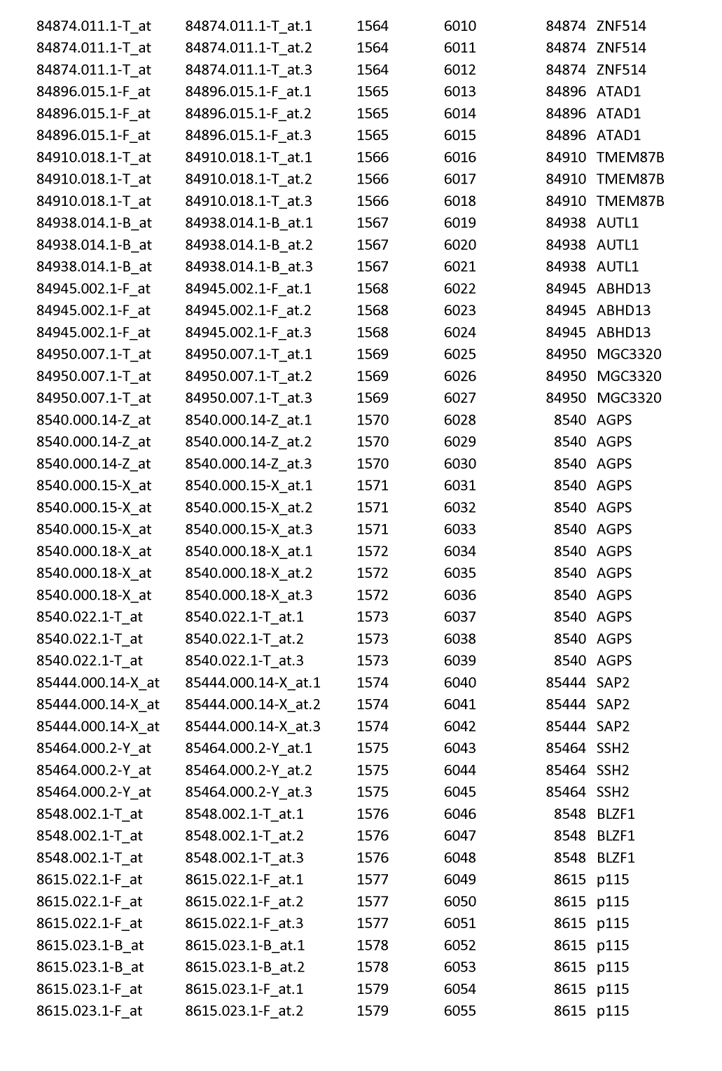

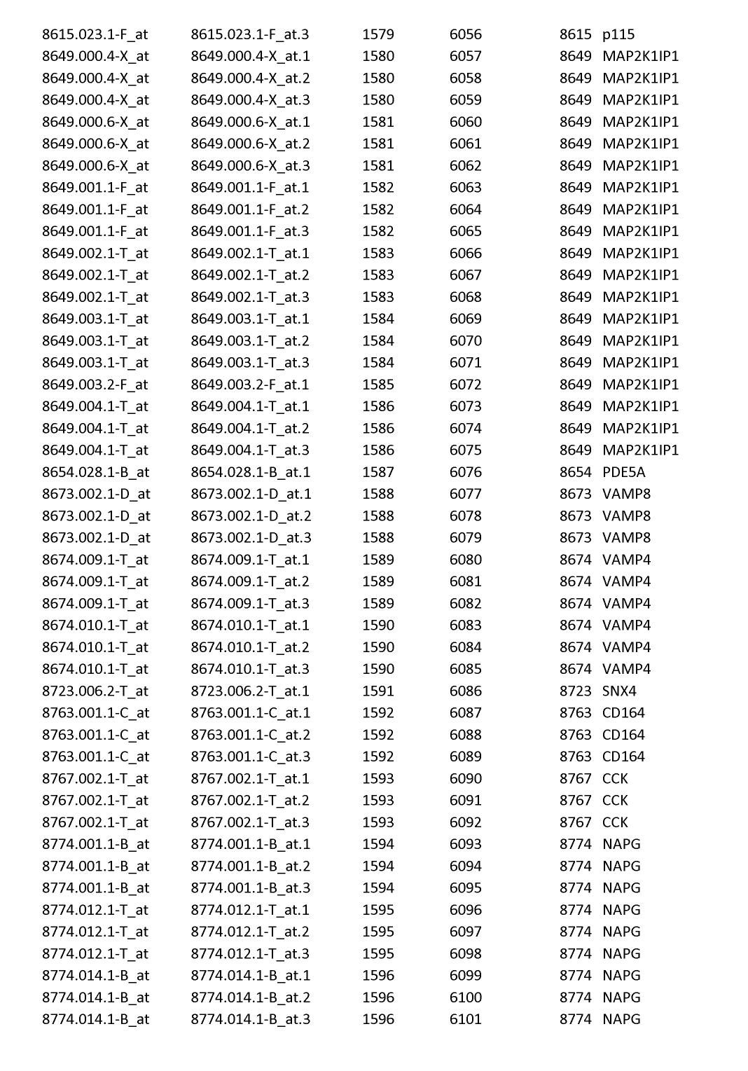

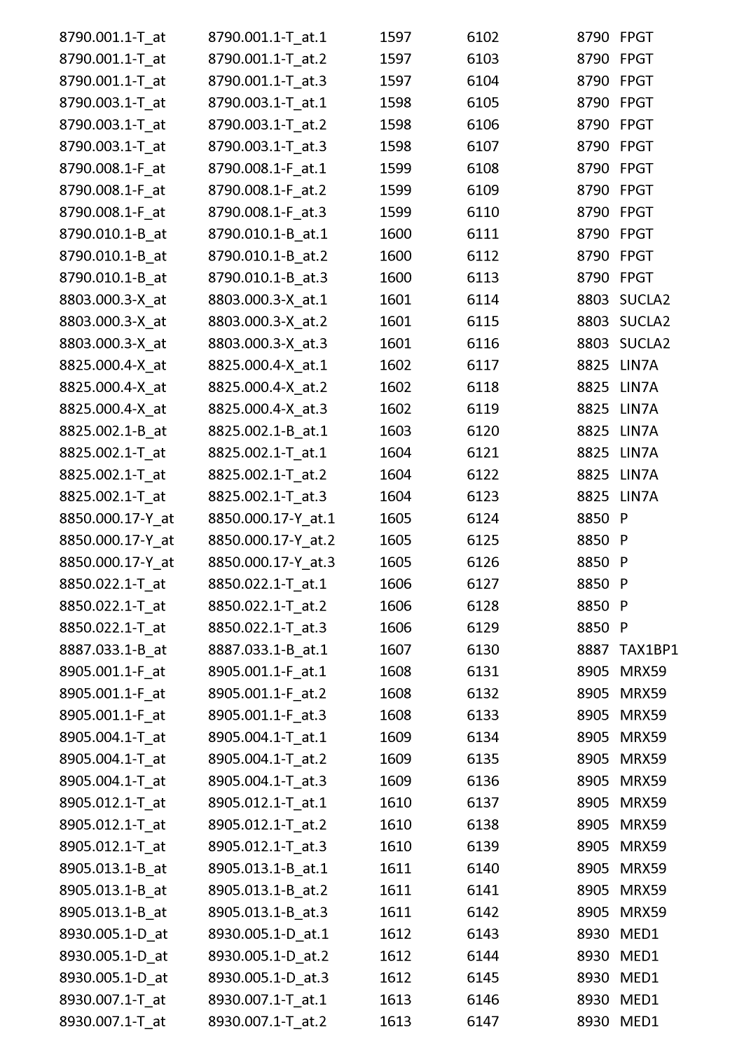

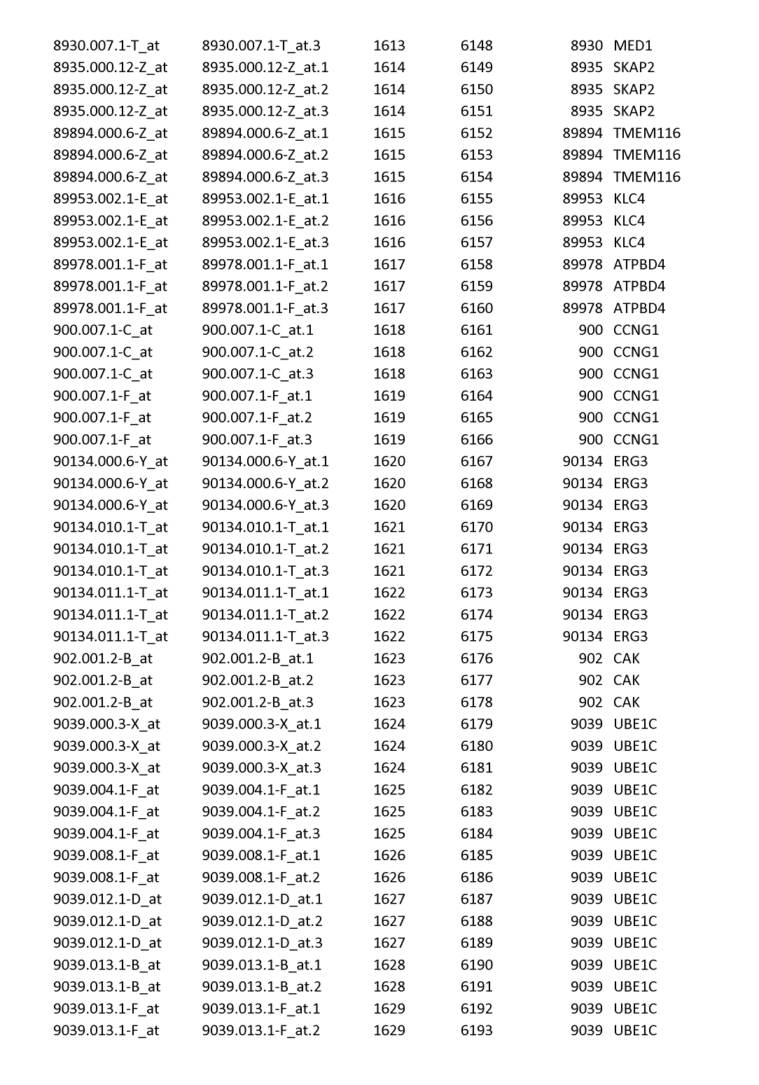

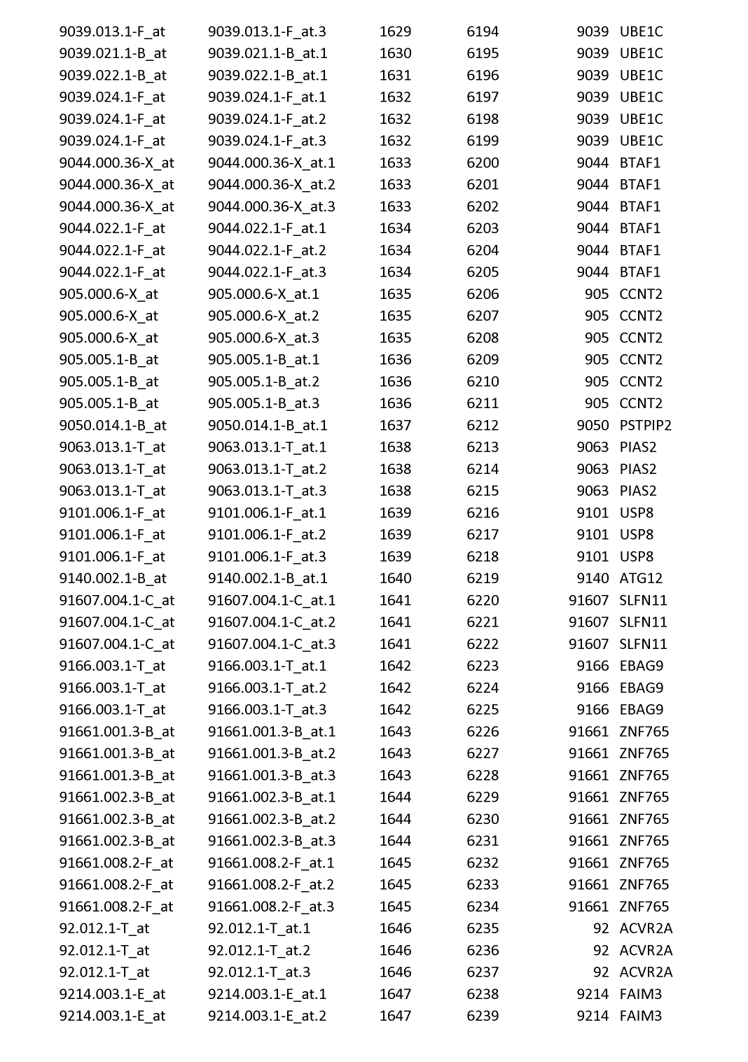

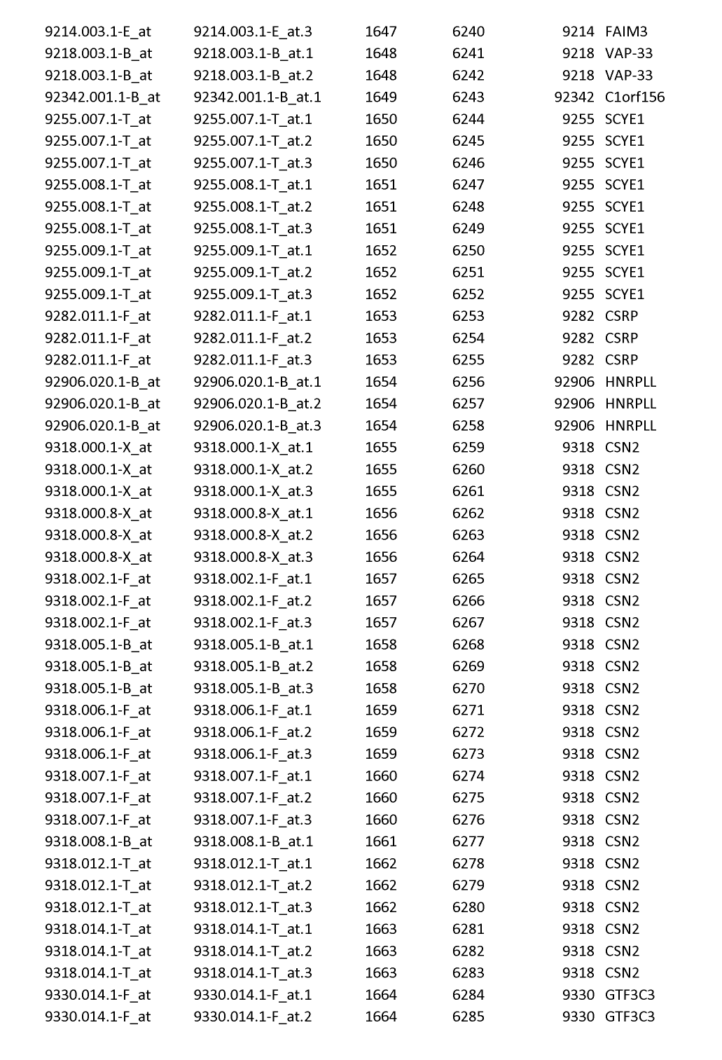

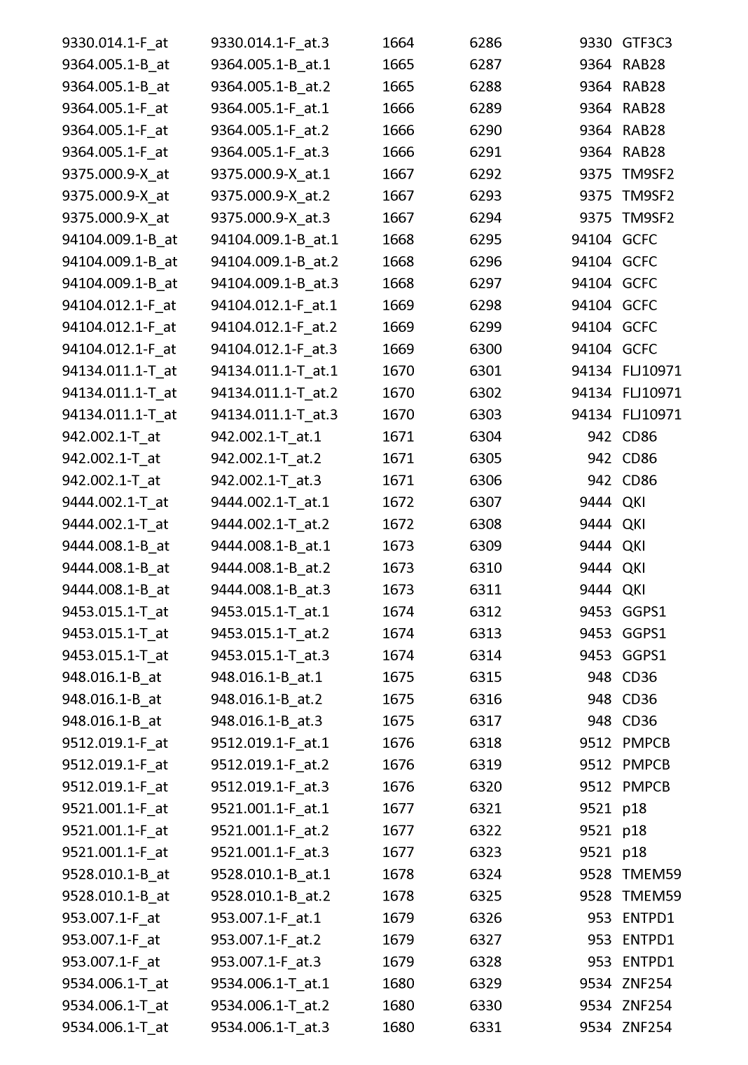

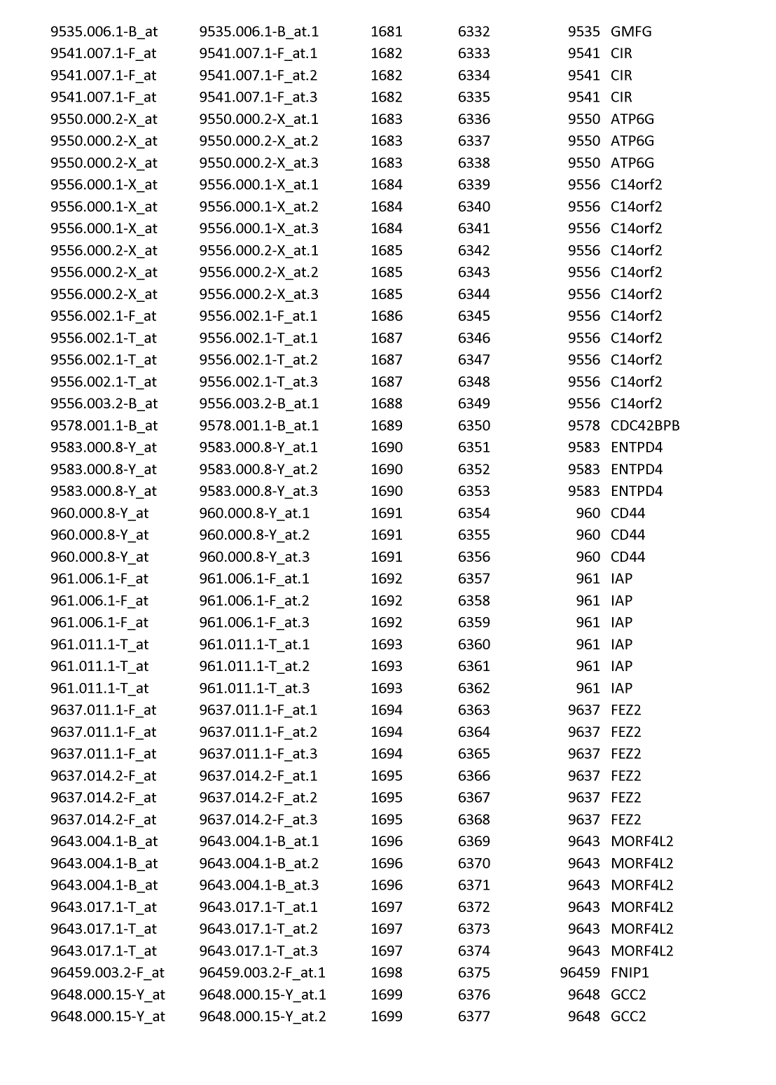

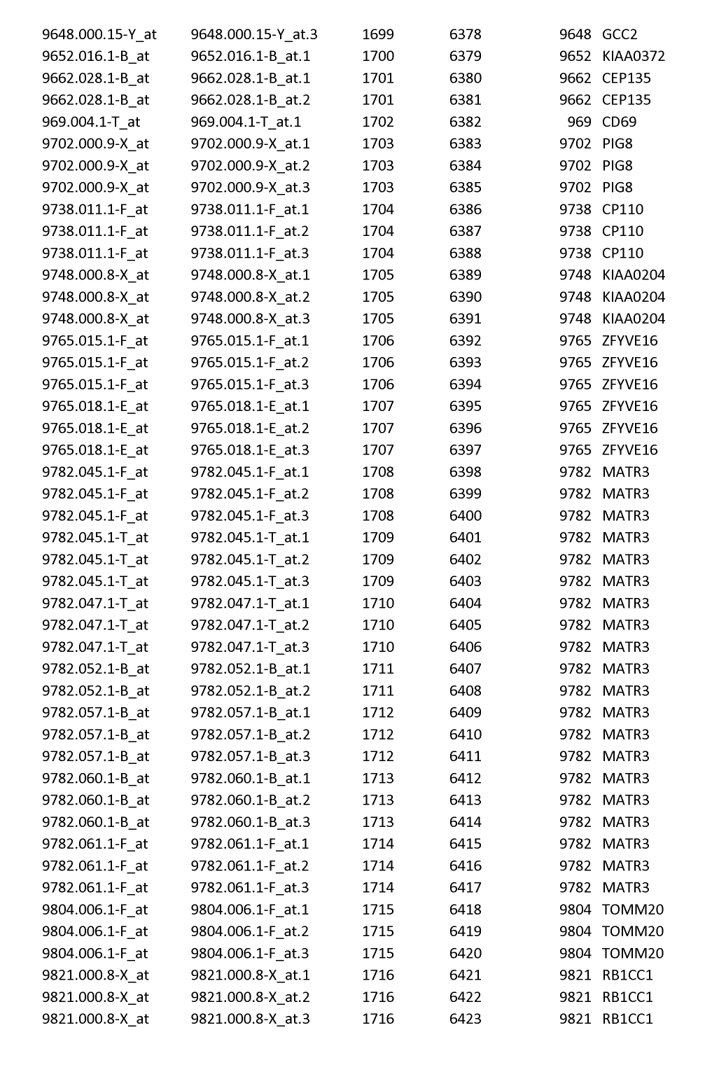

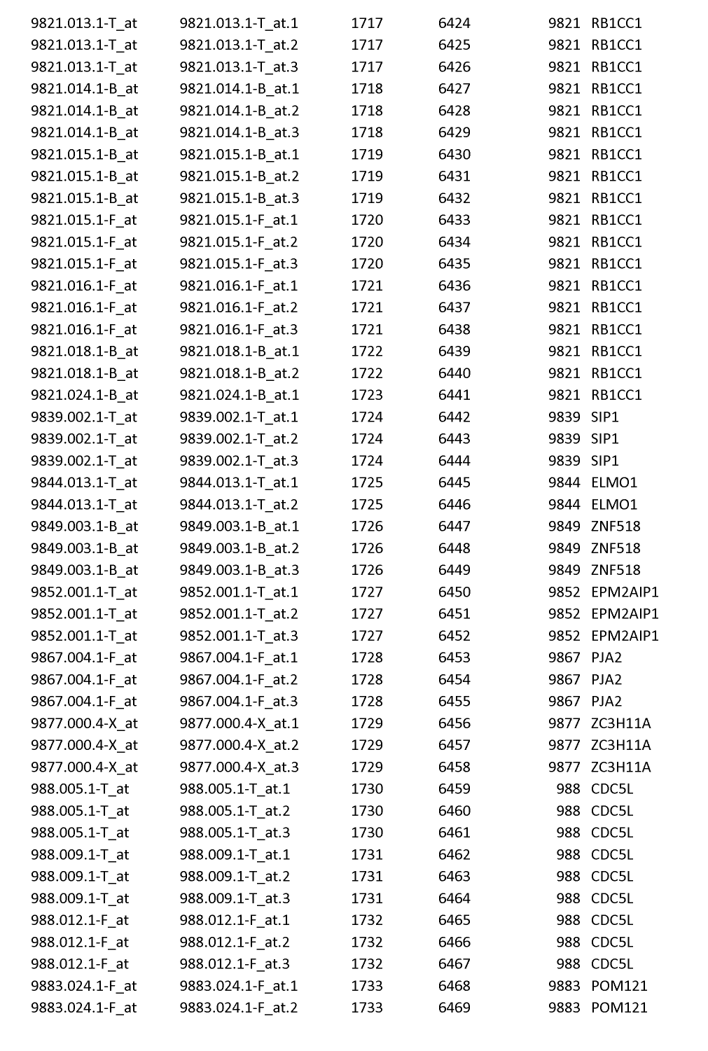

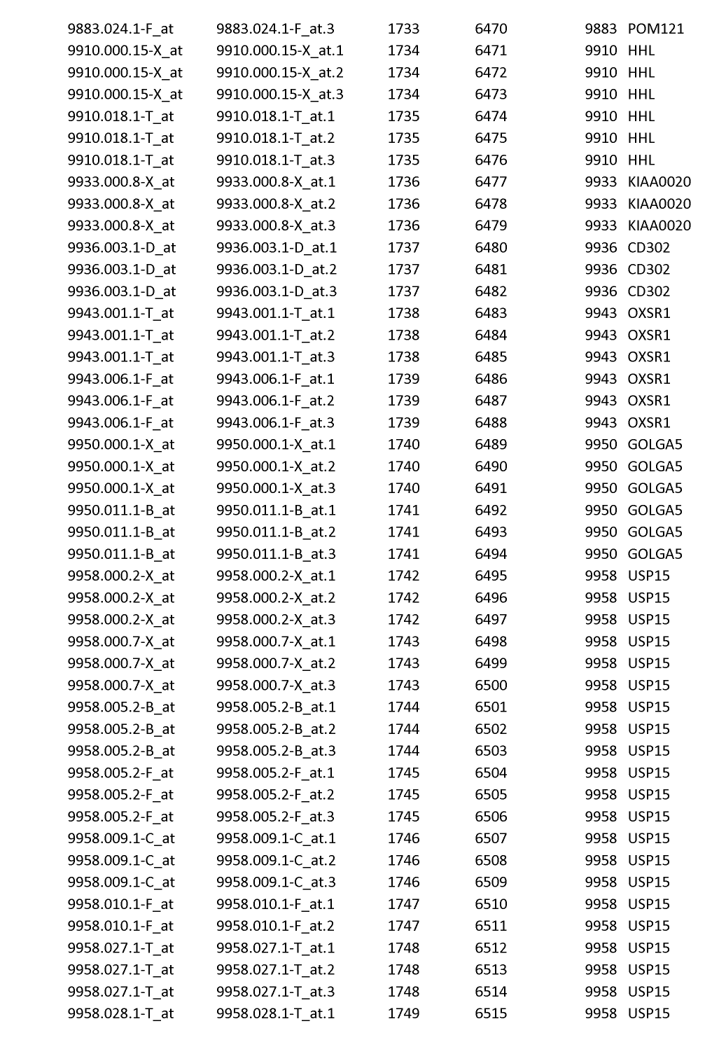

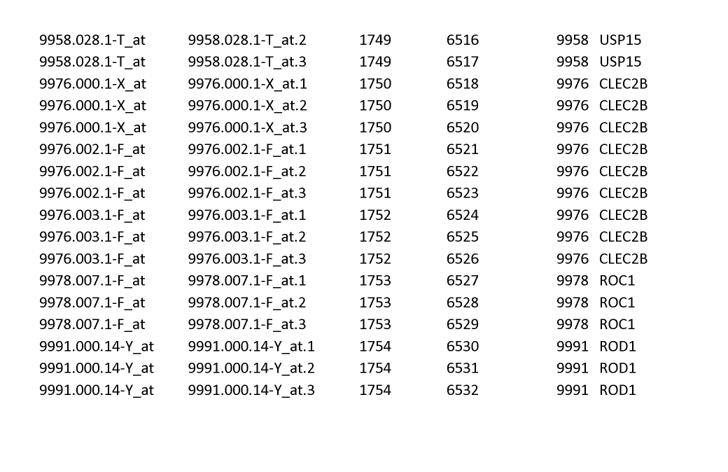

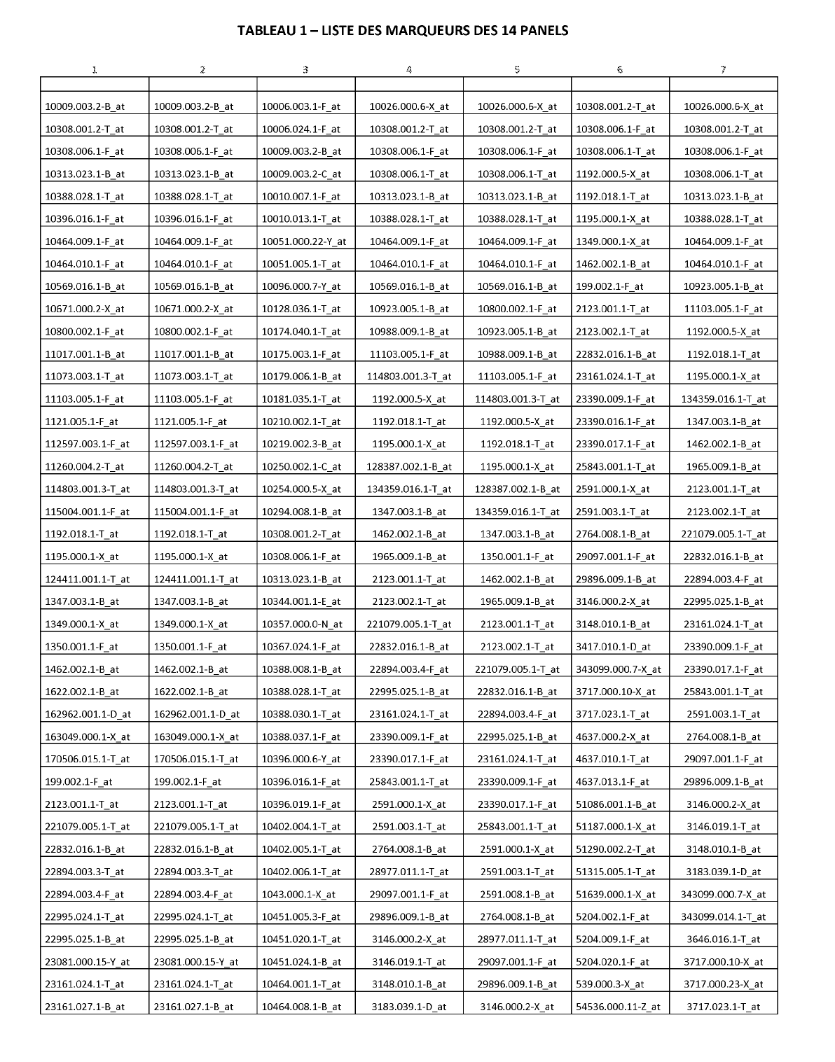

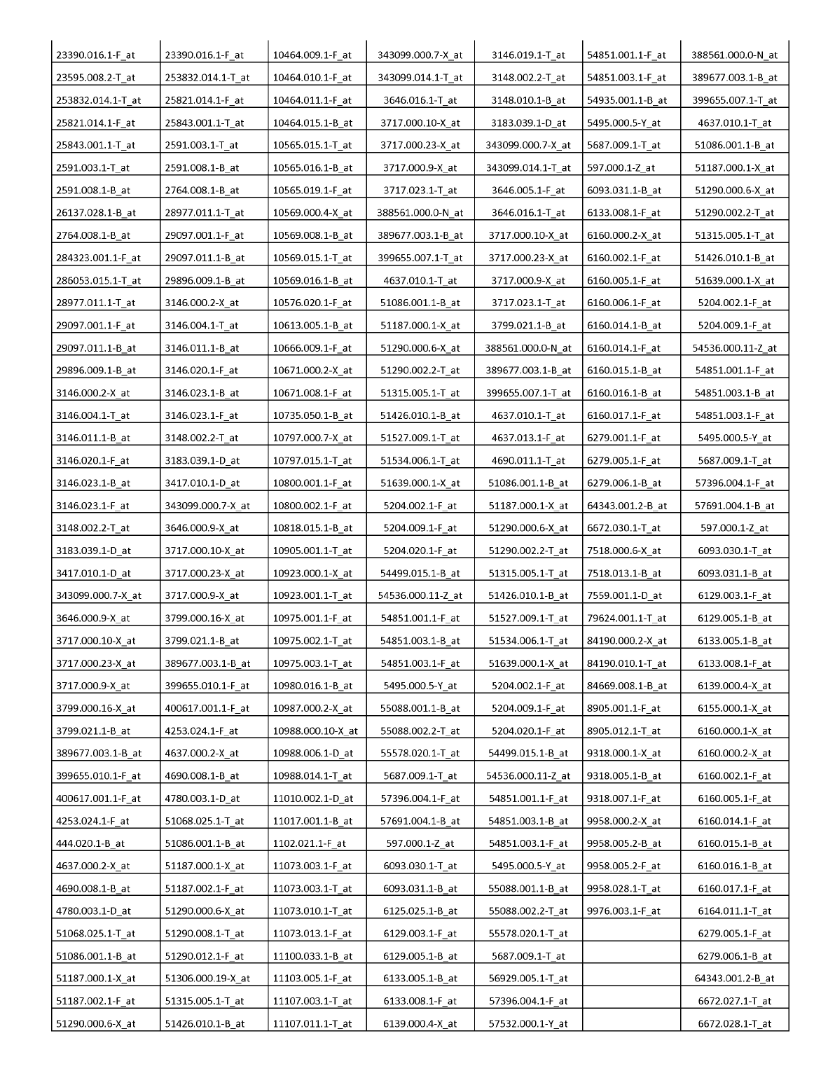

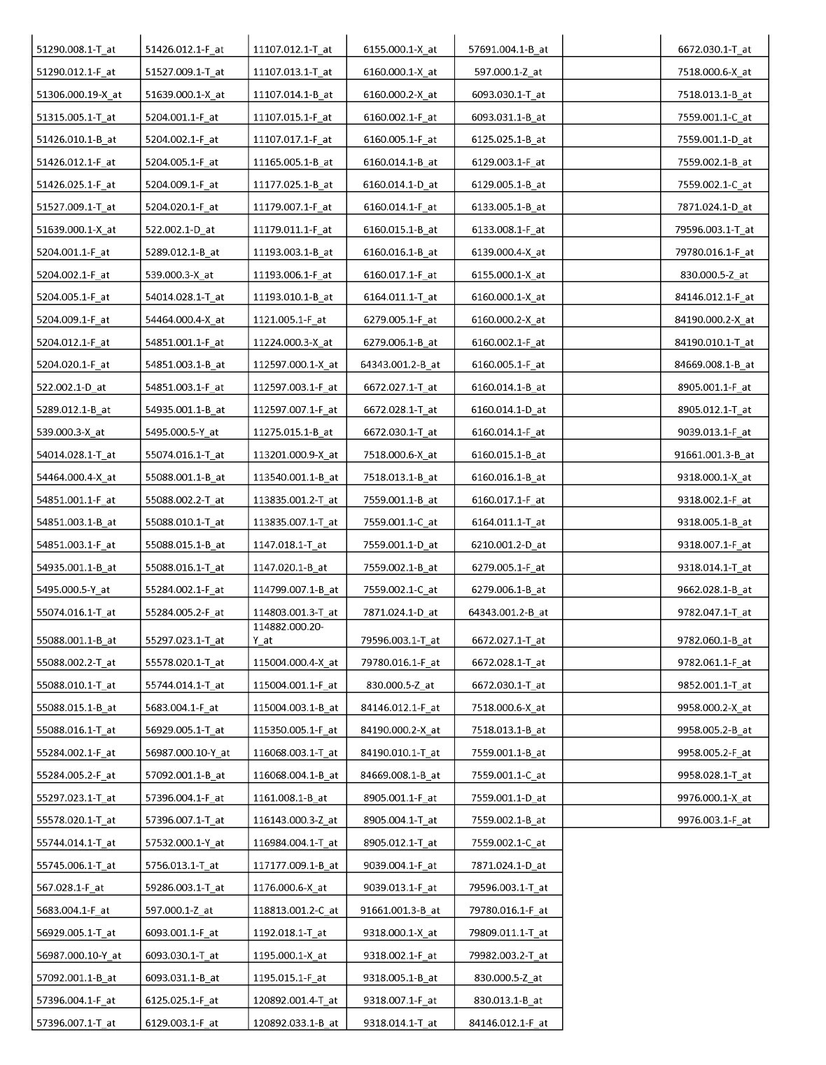

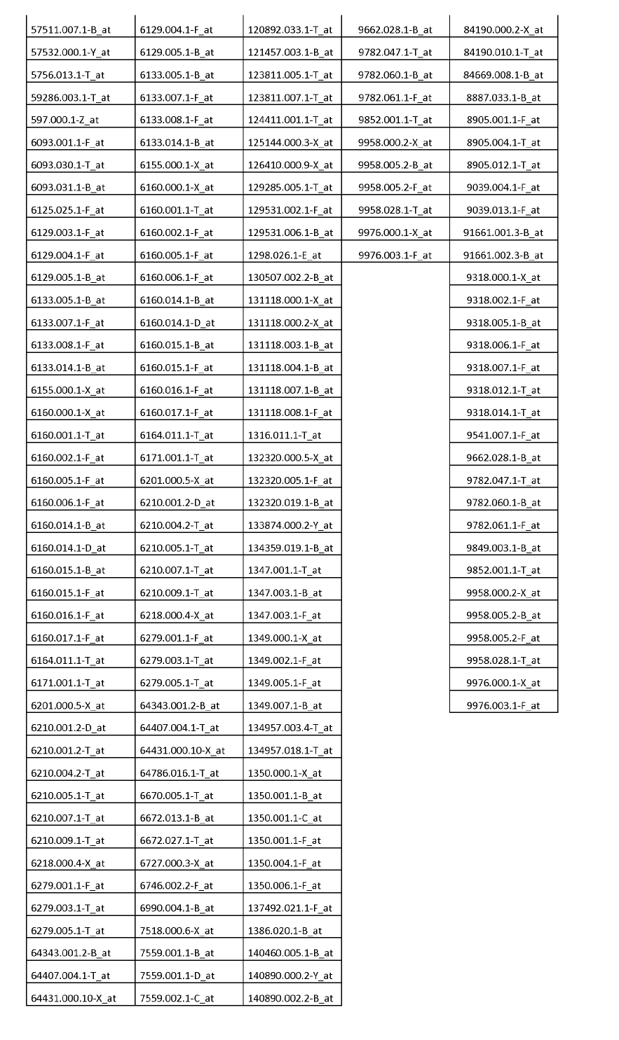

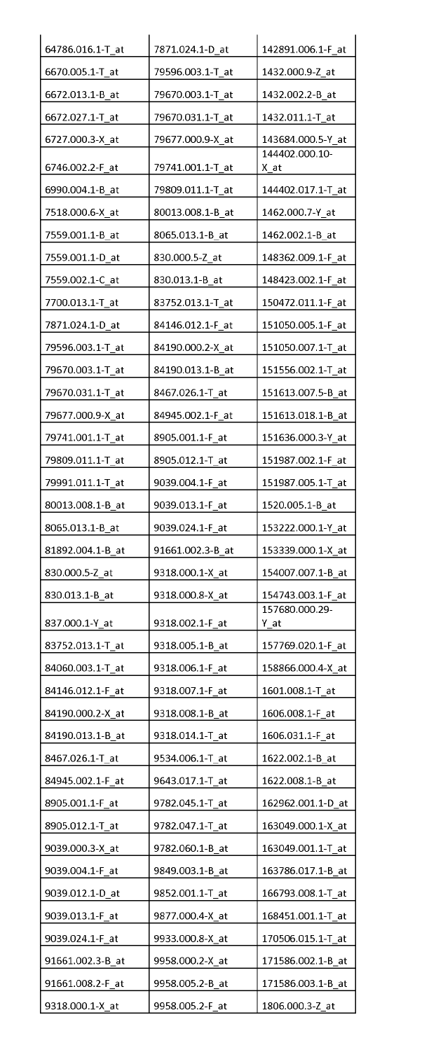

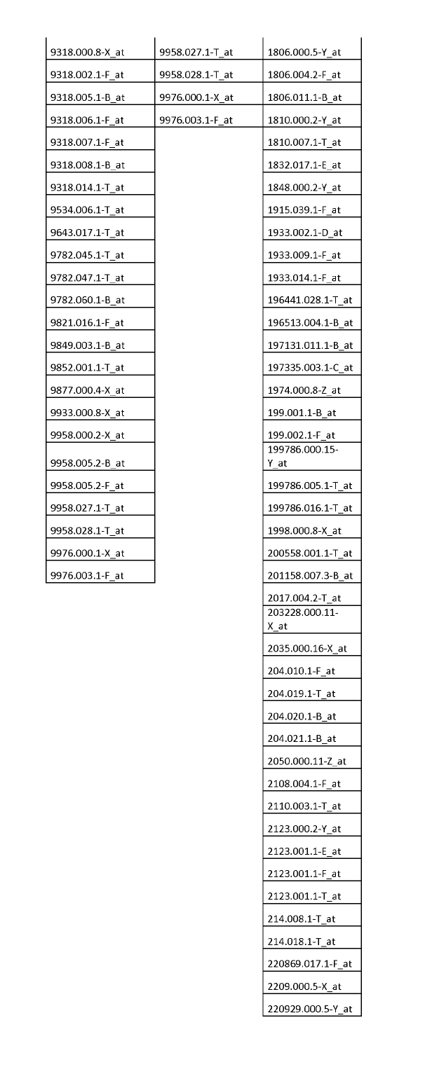

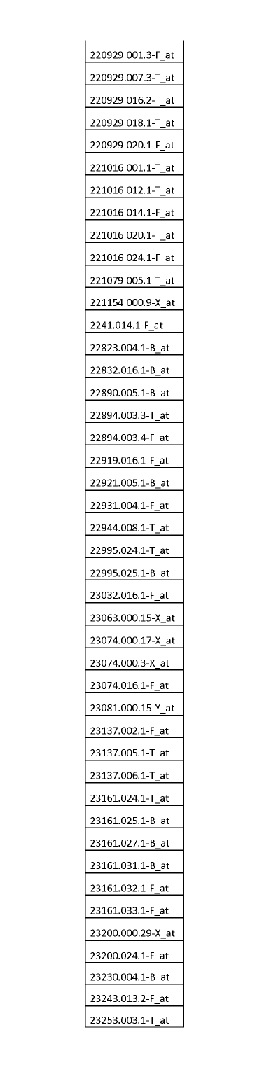

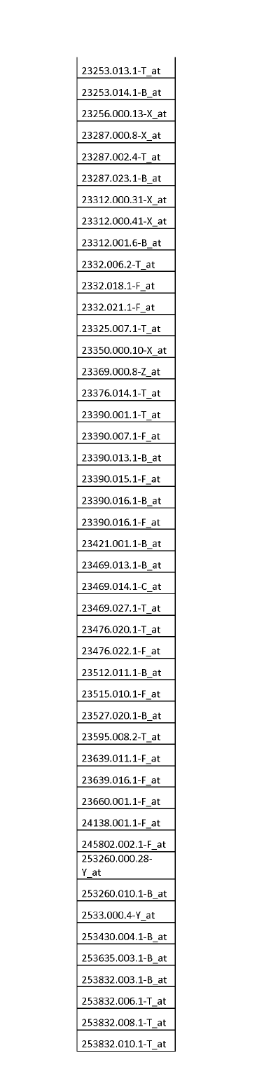

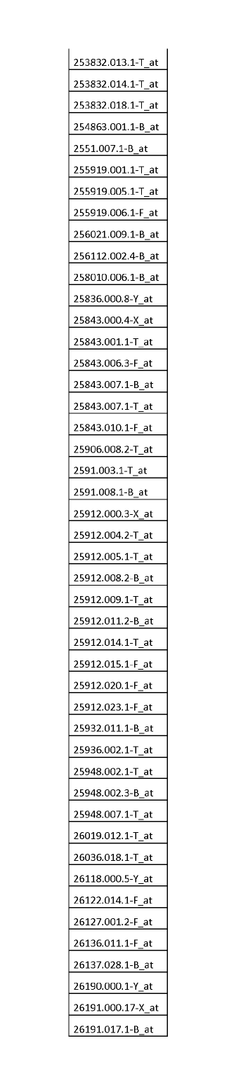

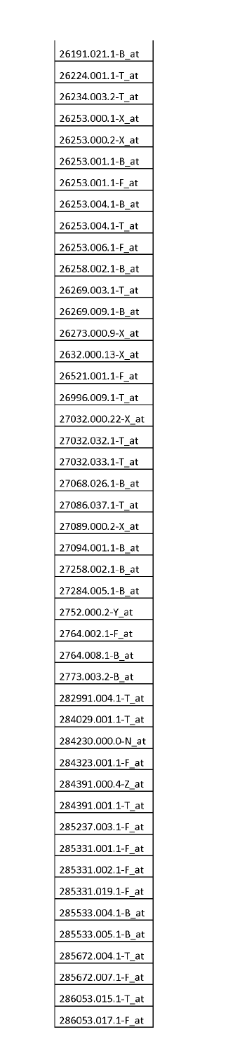

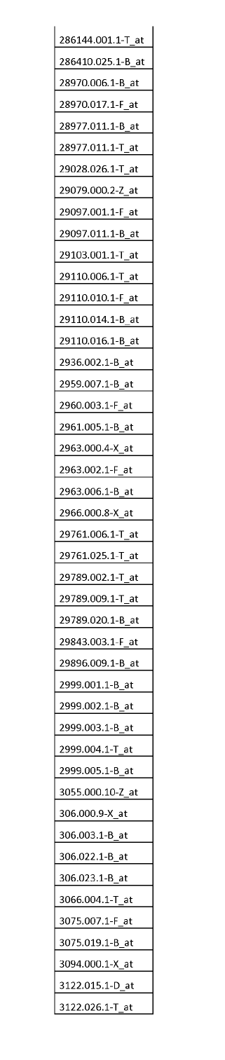

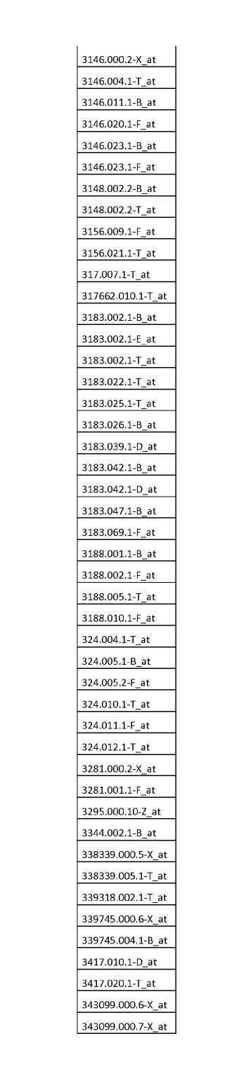

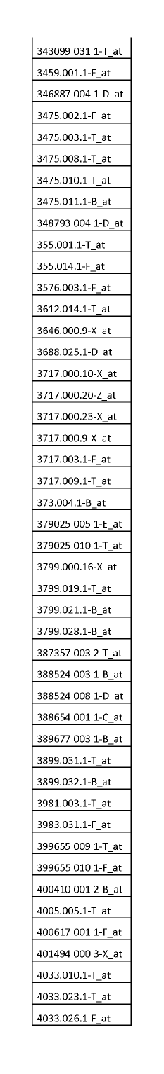

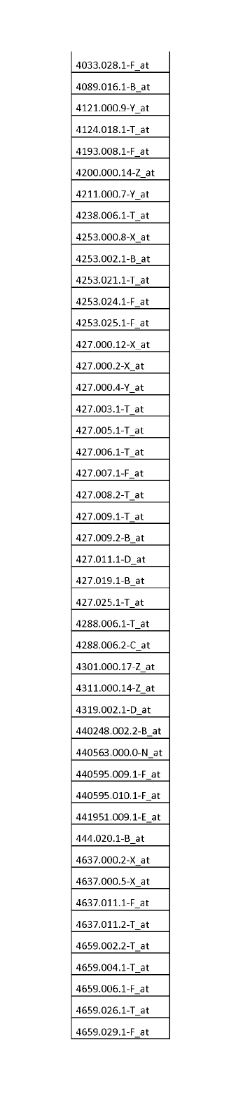

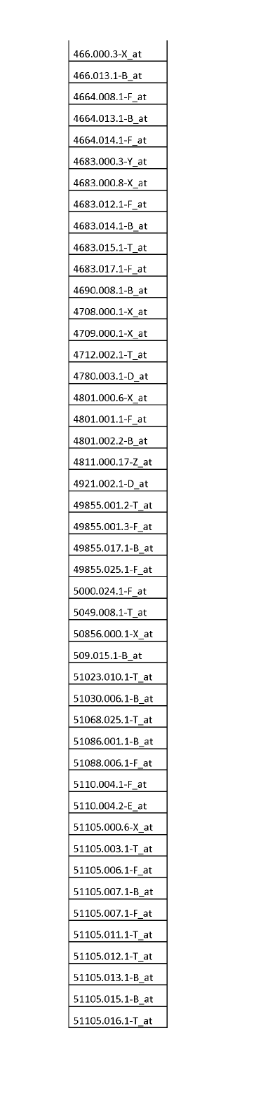

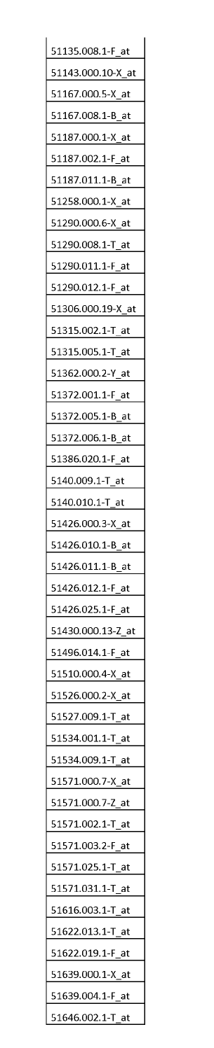

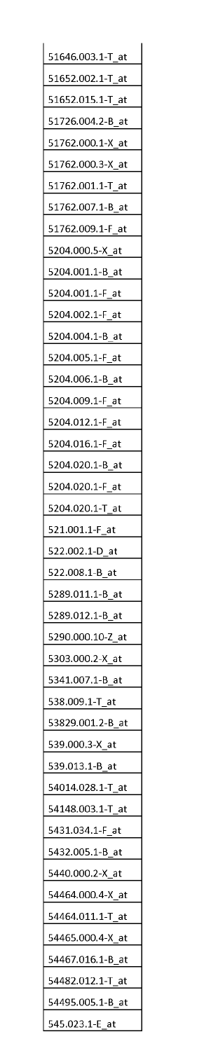

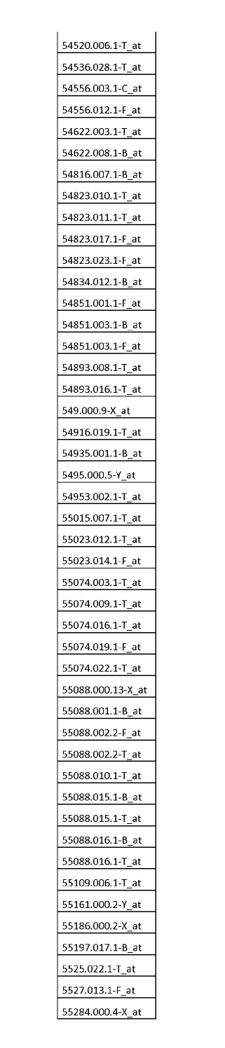

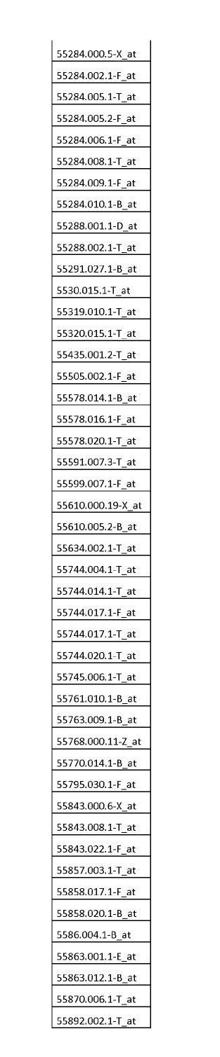

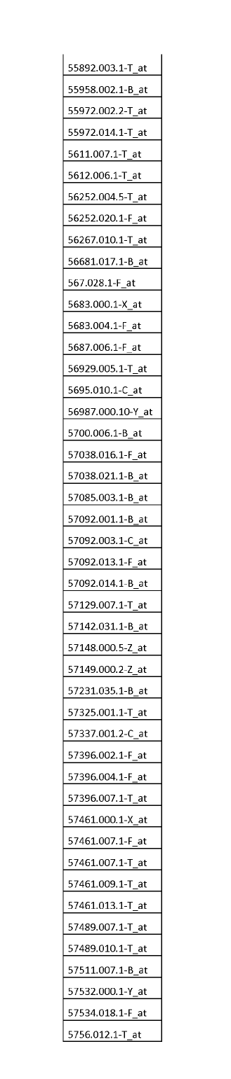

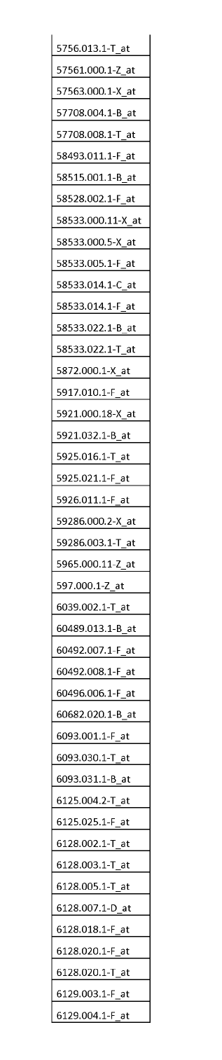

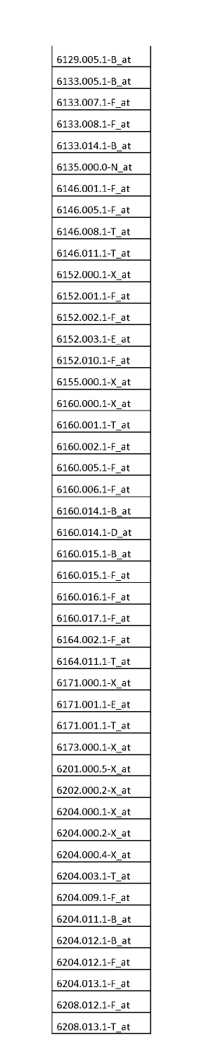

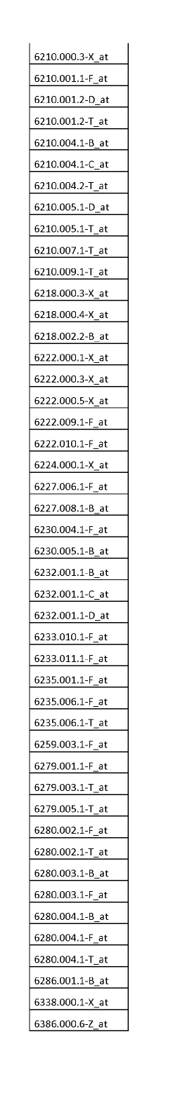

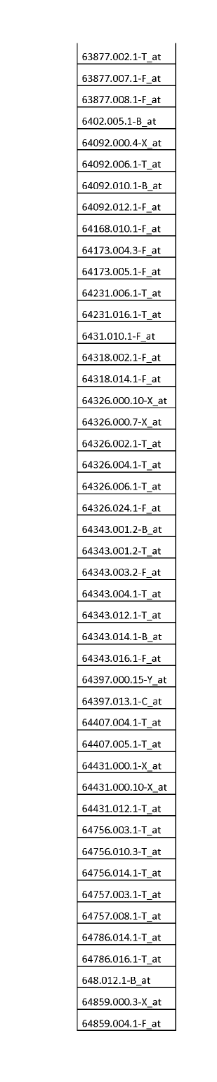

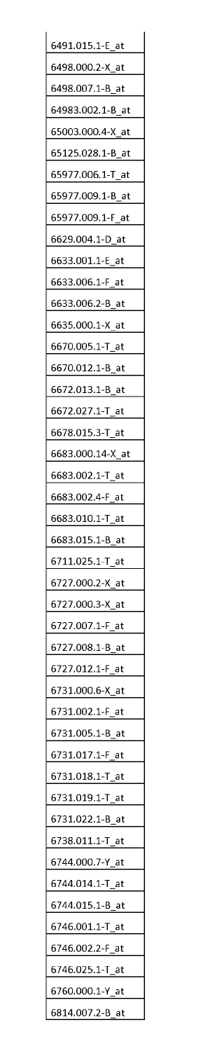

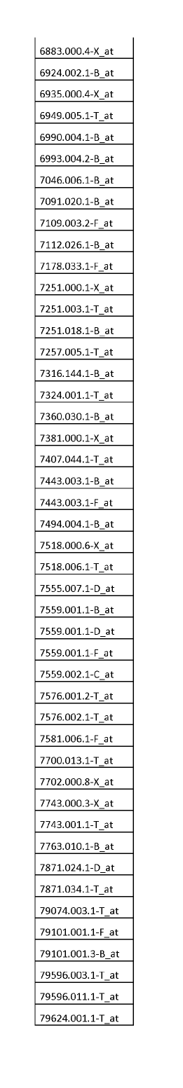

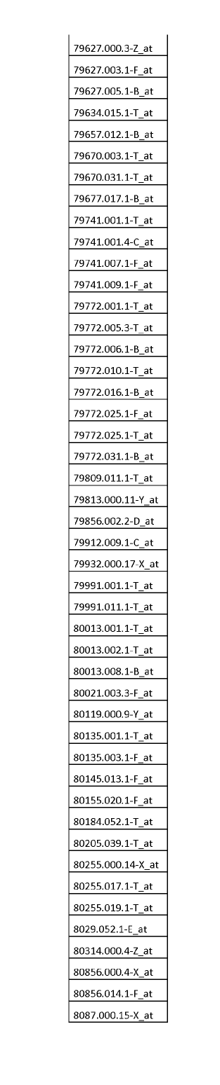

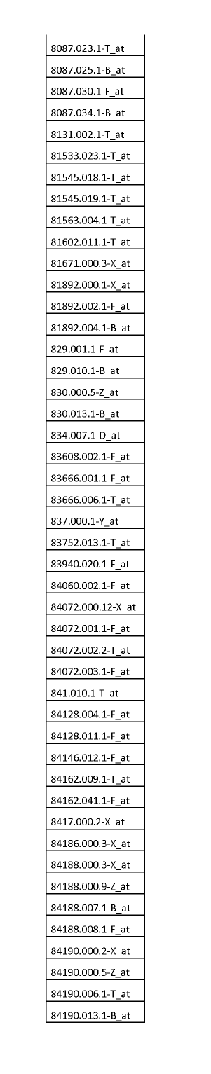

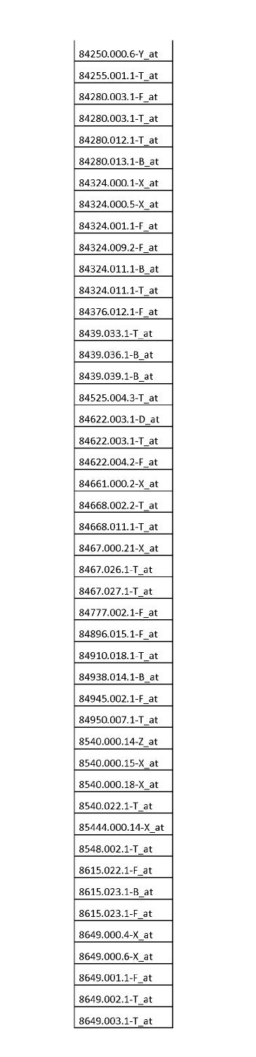

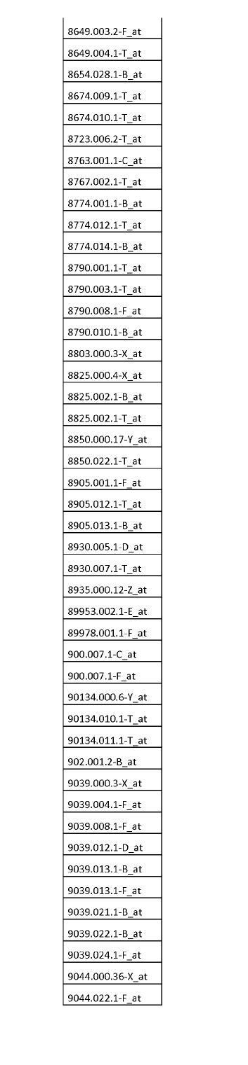

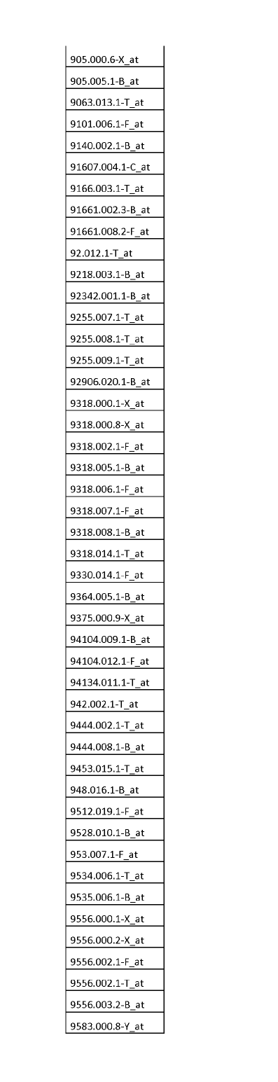

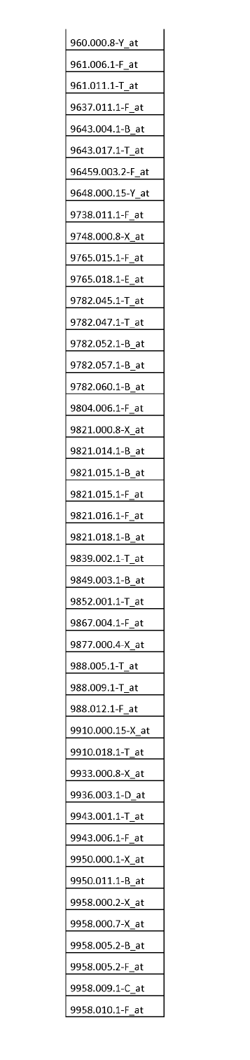

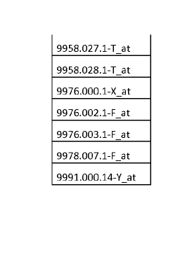

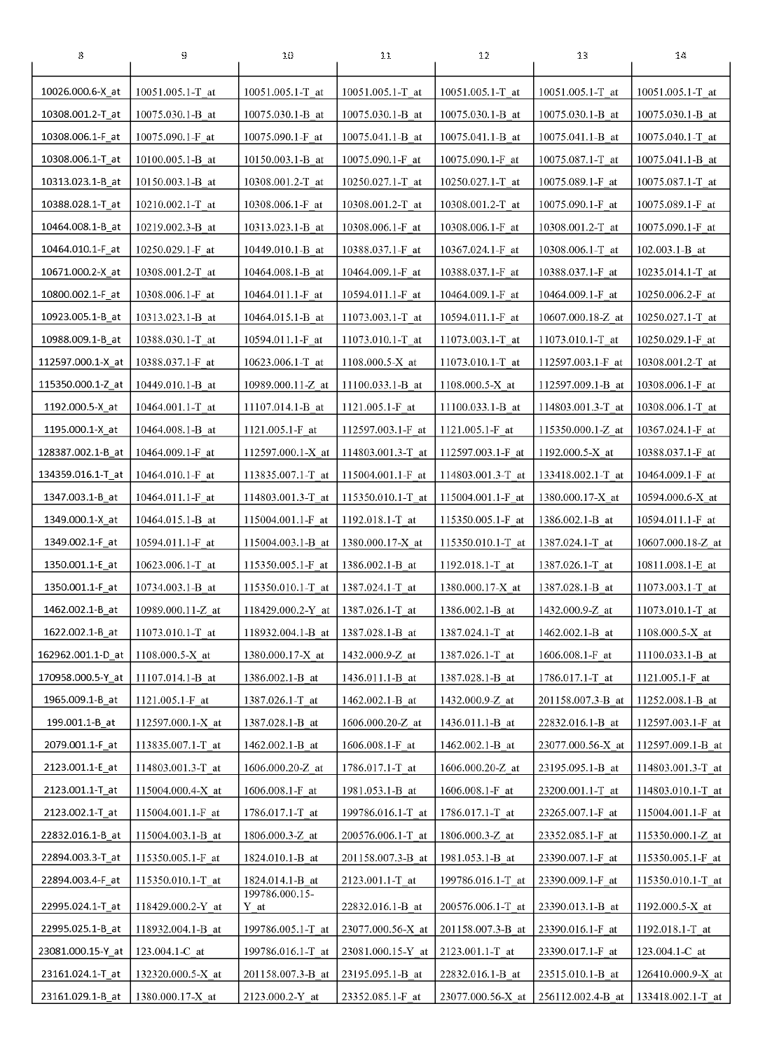

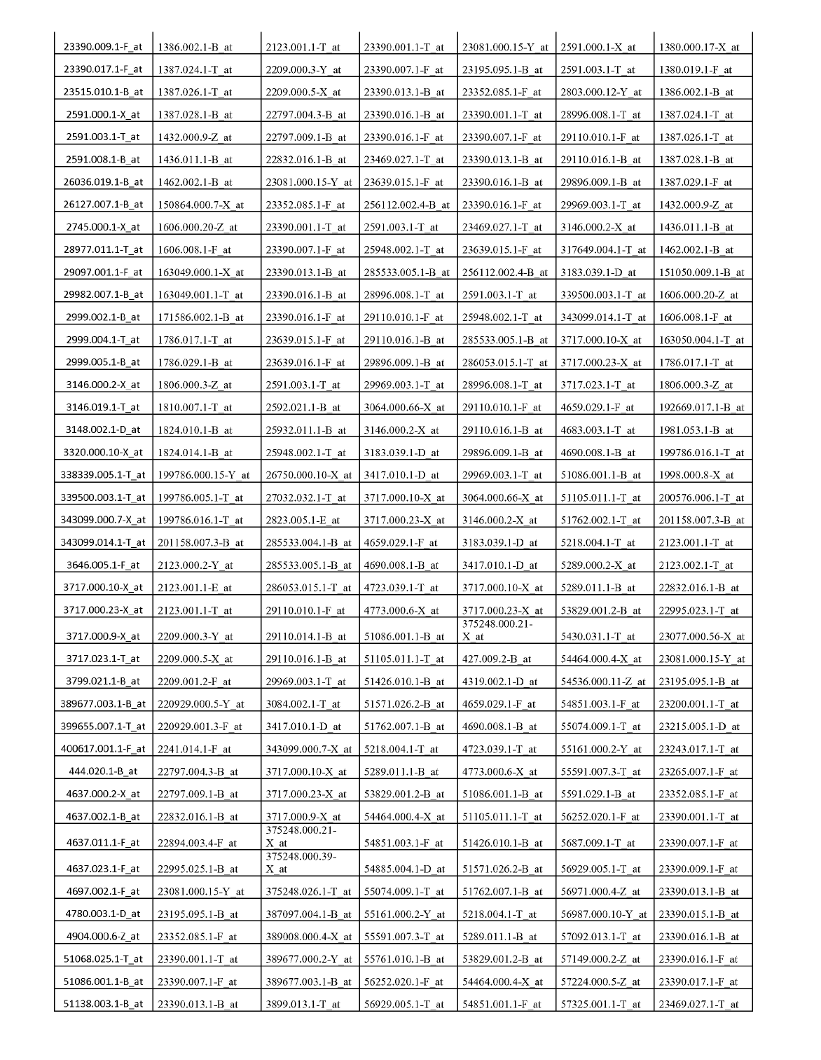

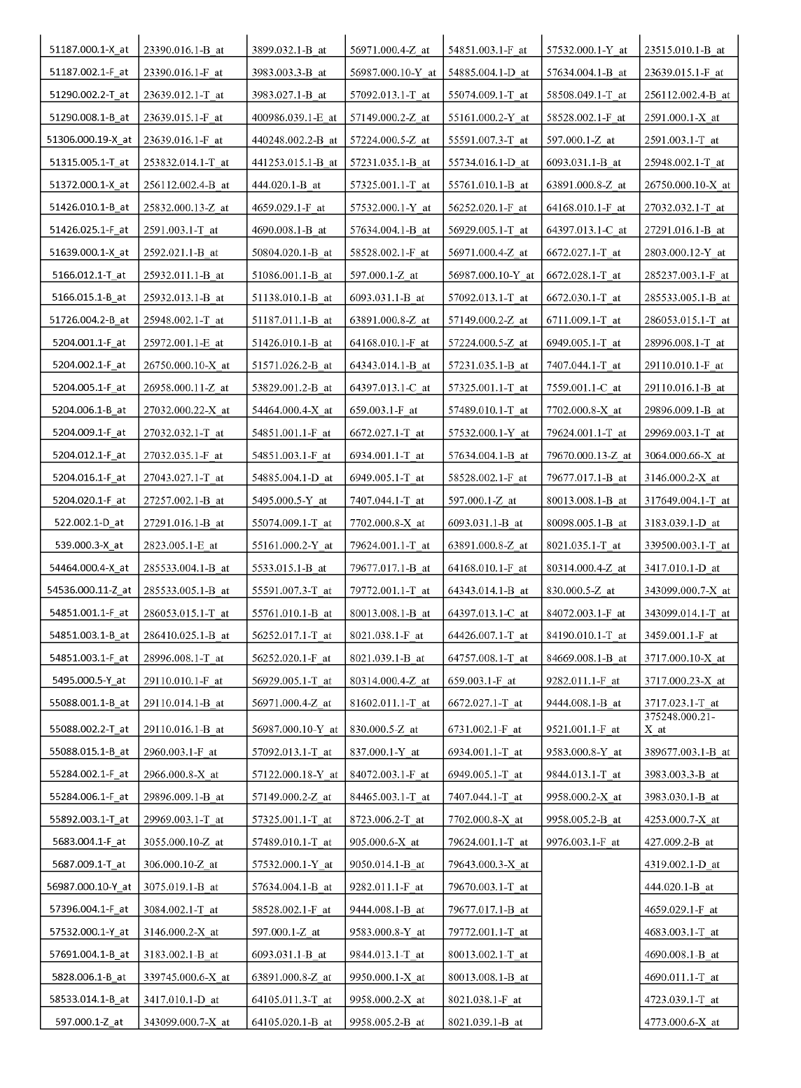

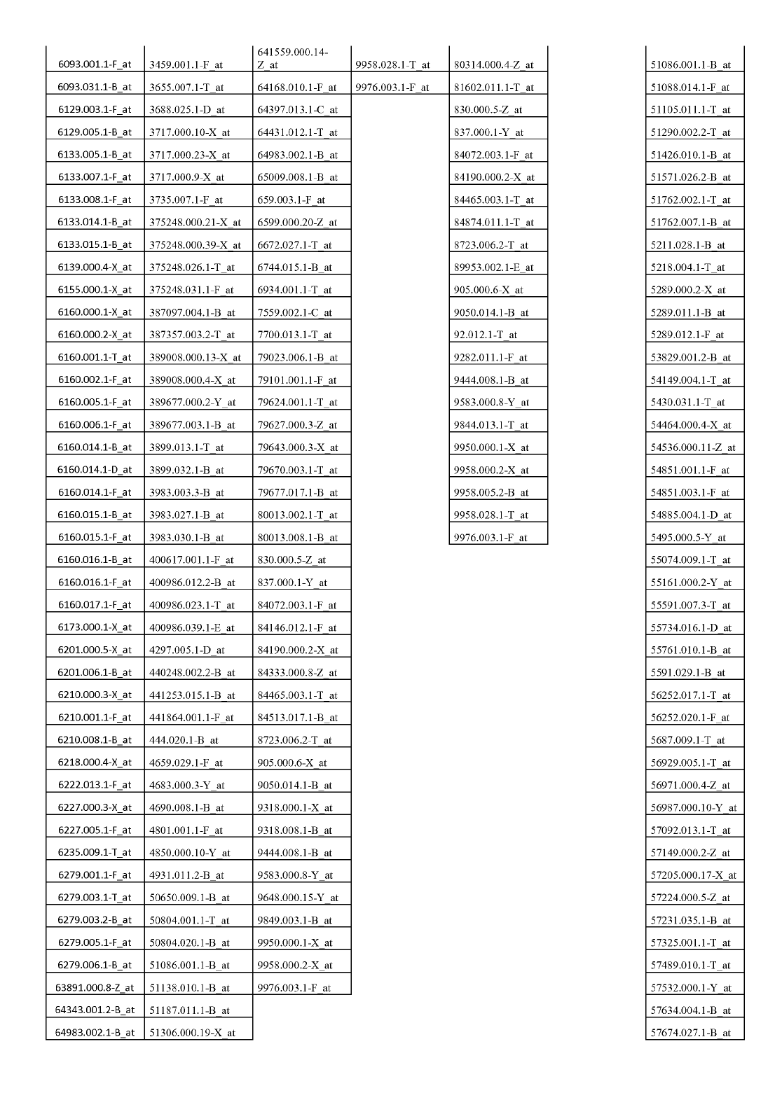

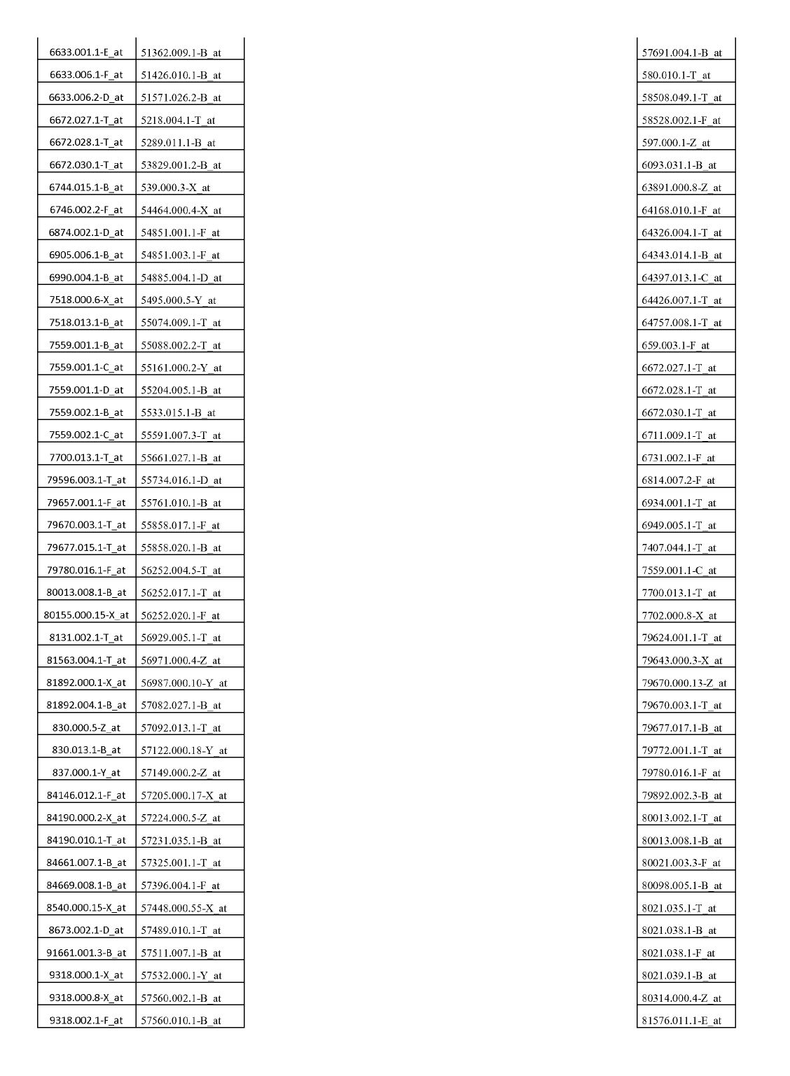

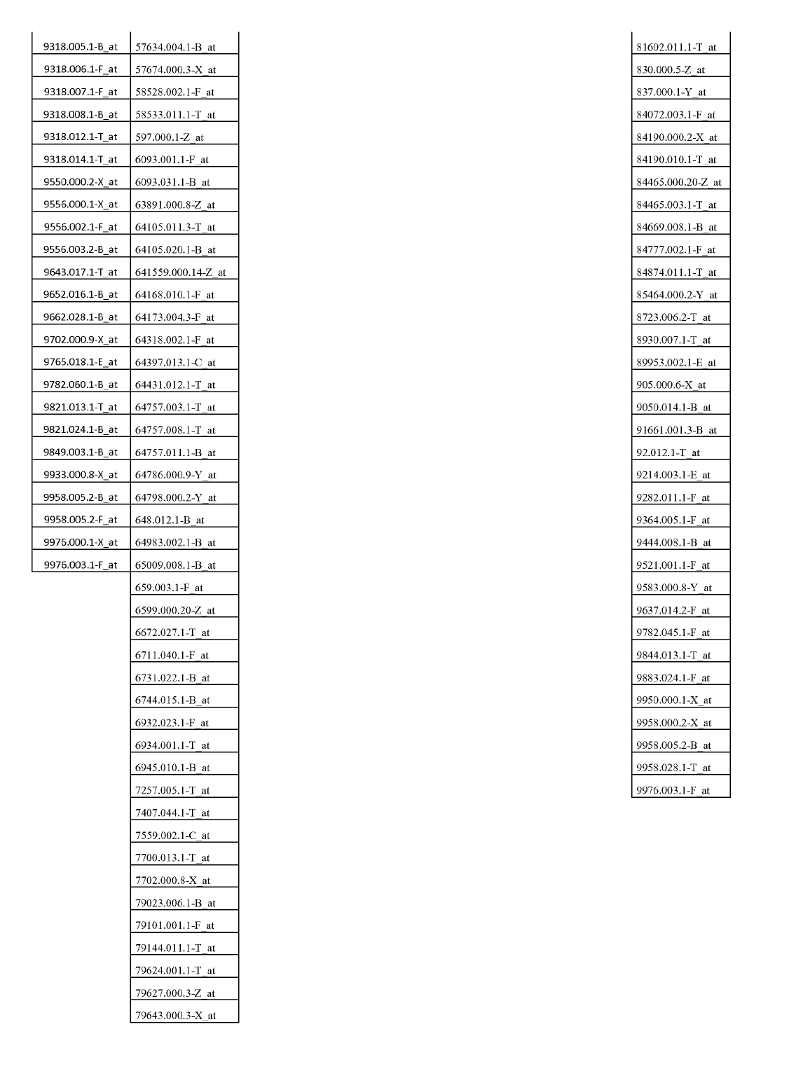

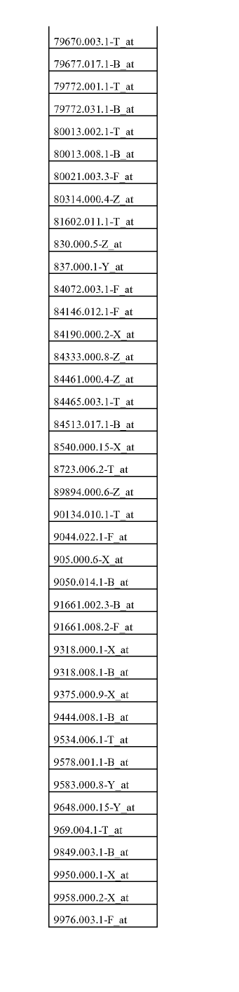

The detection and quantification of the exhaustive expression of transcripts by microarray require the use of a specific configuration of probes. Any reference messenger RNA/splicing variant pair can be modelled as a long isoform/short iso form (FIG. 1A). Thus, a splicing variant associated with an exon jump will be the short isoform compared to the reference variant. A splicing variant comprising a new exon or retaining an intron will be the long isoform compared to the reference variant. Other alternative splicing events (use of 5′ or 3′ cryptic splice sites) can also be modelled in the same manner.

The set of probes necessary to measure the expression of splicing variants is also indicated in FIG. 1A. This set is composed of three traditional exon probes F, T and B, and three exon-exon or exon-intron C, D and E junction probes. Probes F and T measure the expression of both isoforms, probes B, C and D measure the expression of the long isoform and probe E measures the expression of the short form.

In order to design all these probes, it is necessary to identify the splicing events corresponding to human genes, then the “target” regions from which the probes will be designed. The target sequences corresponding to junction probes C, D and E are defined by a length of 30 nucleotides, 15 nucleotides on either side of the junction. It is thus possible to “cover” any junction by probes of 25 nucleotides, for example 11/14, 12/13 or 13/12 (the slash (/) representing the junction region).

The GWSA chip is a microarray capable of providing thoroughly complete coverage of the expression of the human genome by taking into account the existence of splicing variants. 20,649 human genes were selected and then analysed to identify known splicing events and associated potentials. More than 90% of these genes could be associated with such events. Probes as described above were designed from the roughly 140,000 identified splicing events. In addition, the use of probes specific for splicing events corresponding to single exon jumps also makes it possible to predict the existence of such events (FIG. 1B). Moreover, the incorporation of junction probes covering each exon-exon, exon-intron and intron-exon region of each gene also makes it possible to characterise the splicing events that affect the 5′ and 3′ ends of exons (FIG. 1B). Three different probes were designed by target sequence and combined into a probe set. The expression values of individual probes were consolidated in a probe set value. At times, the probe set will contain only 2 or even 1 probe if the restrictions on the target sequence were too constraining GWSA is an individually designed microarray built on the Affymetrix GeneChip® platform with a resolution of 5 microns. The GWSA passed a quality control according to MAQC standards (Microarray Quality Control; Shi et al., Nat Biotechnol. 2006; 24 (9): 1151-61).

1.4. RNA Amplification

50 ng of total RNA was used as matrix for target synthesis using the WT-Ovation™ Pico RNA Amplification System kit (NuGen, San Carlos, Calif.). The FL-Ovation™ cDNA Biotin Module (NuGen, San Carlos, Calif.) was then used to carry out the fragmentation of 5 μg of cDNA amplified as well as biotin labelling. The various steps were performed by following the manufacturer's instructions. Each amplification/fragmentation/marking series contained equal numbers of samples from AD patients and controls.

The quality and the quantity of complementary DNA extracted were evaluated by performing electrophoregrams using Agilent's 2100 Bioanalyser and a NanoChip RNA 6000 kit (Agilent Technologies, Santa Clara, Calif.).

1.5. Hybridisation on GWSA Chips

5 μg of cDNA amplified and marked with biotin are used by hybridisation. The standard methods recommended by Affymetrix (Affymetrix, Santa Clara, Calif.) were applied for hybridisation of the targets to the GWSA microarray. The DNA chips were then washed and specific hybridisation revealed by following Affymetrix's recommendations. Hybridisation signals were detected using the GeneChip® 3000 7G scanner.

1.6. Data Retrieval and Standardisation

.CEL files obtained after scanning the chips were then imported into Partek Genomic Suites™ (Partek Incorporated, St. Louis, Mich.) for, an adjustment of background signal as a function of the composition in GC of the oligonucleotide probes, correction of background signal by RMA, a quantile normalization of the arrays, and consolidation of expression values measured for each oligonucleotide in the probe set (1 455 607 PS).

The data were then filtered with respect to the expression values of the probe sets, the threshold expression level was set at 6.2 (log 2 scale) while being based on the distribution frequency of the expression value on the complete set of probes (361 299 PS) and with respect to variance, the threshold was set at 0.3 (299 577 PS).

### Example 2

### Selection of Signatures

A training group of discriminating signatures were used for supervised clustering analyses using binary classification algorithms and 5- or 10-fold cross validations (Partek Genomic Suites™). Nonparametric tests (1-way ANOVA) were performed for the analysis of the expression values obtained by the probe set and were used as a filtering tool for the selection of variables that integrated in the signature. Optimal classification models by training group were then applied to the test group for the identification of the performance of the signature, namely specificity (% proper classification of control patients) and sensitivity (% proper classification of AD patients).

The models were tested and the performance of each model was evaluated on all of the training groups. Fourteen signatures with the highest performance averages were selected to discriminate between the patients suffering from Alzheimer's disease and the non-demented control patients. The list of the markers composing these panels 1-14 is given in table 2. The performance of these panels in the first studies are summarised in the table below:

| Models-panel | AD % | CT % | Performance | PPV | NPV |
|---|---|---|---|---|---|
| AD vs CT-1-240v | 76.90% | 79.60% | 78.30% | 78.43% | 78.18% |
| AD vs CT-2-220v | 73.10% | 79.60% | 76.42% | 77.55% | 75.44% |
| AD vs CT-3-1460v | 75.00% | 77.80% | 76.42% | 76.47% | 76.36% |
| AD vs CT-4-140v | 73.10% | 77.80% | 75.47% | 76.00% | 75.00% |
| AD vs CT-5-160v | 73.10% | 75.90% | 74.53% | 74.51% | 74.55% |
| AD vs CT-6-80v | 73.10% | 75.90% | 74.53% | 74.51% | 74.55% |
| AD vs CT-7-120v | 71.20% | 75.90% | 73.58% | 74.00% | 73.21% |
| AD vs CT-8-240v | 80.80% | 77.80% | 79.25% | 77.80% | 80.80% |
| AD vs CT-9-300v | 78.80% | 77.80% | 78.30% | 77.37% | 79.21% |
| AD vs CT-10-170v | 76.90% | 79.60% | 78.30% | 78.40% | 78.16% |
| AD vs CT-11-130v | 76.90% | 75.90% | 76.42% | 75.45% | 77.33% |
| AD vs CT-12-150v | 76.90% | 74.10% | 75.47% | 74.09% | 76.91% |
| AD vs CT-13-120v | 75.00% | 75.90% | 75.47% | 74.98% | 75.92% |
| AD vs CT-14-250v | 75.00% | 75.90% | 75.47% | 74.98% | 75.92% |

### Example 3

### Demonstration of an Expression Profile to Discriminate Control Patients from Patients Suffering from Alzheimer's Disease (AD) from Blood Samples

Expression of the entire human transcriptome, representing approximately 21,000 genes, was analysed and compared between AD and Non Demented Control (NDT) patients using a GWSA microarray. All of the analyses mentioned in example 2 revealed a set of 1754 relevant target sequences (see table 1, SEQ ID NOS.: 1-1754).

The inventors then studied the simultaneous expression of probe sets associated with subsets of these 1754 sequences to obtain specific expression profiles for Alzheimer's disease.

The performance of these signatures was evaluated on a test set (validation set) of individuals not having taken part in the definition of this signature and recruited in a blind study. Performance values are described in example 2.

Signature analysis on the level of gene revealed that 10 genes are common to all of the 14 panels. The list of corresponding genes is given below (panel 15).

| Enter ID | Symbol | Description |
|---|---|---|
| 10308 | ZNF267 | zinc finger protein 267 |
| 1462 | CSPG2 | chondroitin sulfate proteoglycan 2 (versican) |
| 22832 | KIAA1009 | kiaa1009 |
| 2591 | GALNT3 | udp-n-acetyl-alpha-d-galactosamine |
| 3717 | JAK2 | Janus kinase 2 (a protein tyrosine kinase) |
| 51086 | TNNI3K | tnni3 interacting kinase |
| 54851 | ANKRD49 | ankyrin repeat domain 49 |
| 597 | BCL2A1 | bcl2-related protein a1 |
| 6093 | ROCK1 | rho-associated, coiled-coil containing protein |
| | | kinase 1 |
| 9976 | CLEC2B | c-type lectin domain family 2, member b |

A preferred example of all probes for detecting Alzheimer's disease thus comprises:
from 1 to 3 probes, overlapping or not, each comprising all or part of sequence SEQ ID NO: 34 or of a sequence complementary thereto;
from 1 to 3 probes, overlapping or not, each comprising all or part of sequence SEQ ID NO: 230 or of a sequence complementary thereto;
from 1 to 3 probes, overlapping or not, each comprising all or part of sequence SEQ ID NO: 341 or of a sequence complementary thereto;
from 1 to 3 probes, overlapping or not, each comprising all or part of sequence SEQ ID NO: 454 or of a sequence complementary thereto;
from 1 to 3 probes, overlapping or not, each comprising all or part of sequence SEQ ID NO: 664 or of a sequence complementary thereto;
from 1 to 3 probes, overlapping or not, each comprising all or part of sequence SEQ ID NO: 811 or of a sequence complementary thereto;
from 1 to 3 probes, overlapping or not, each comprising all or part of sequence SEQ ID NO: 951 or of a sequence complementary thereto;
from 1 to 3 probes, overlapping or not, each comprising all or part of sequence SEQ ID NO: 1127 or of a sequence complementary thereto;
from 1 to 3 probes, overlapping or not, each comprising all or part of sequence SEQ ID NO: 1136 or of a sequence complementary thereto; and
from 1 to 3 probes, overlapping or not, each comprising all or part of sequence SEQ ID NO: 1752 or of a sequence complementary thereto.

### Example 4

### Demonstrating an Expression Profile to Discriminate Control Patients from Patients Suffering from Alzheimer's Disease (AD) from Blood Samples

Analysis of signatures on the gene level made it possible to select 17 markers common to the 5 panels presenting the best performance. The list of these 17 markers (which includes the 10 markers of example 3), is given below (panel 16).

| 17 PS ADvsC | SEQ ID |
|---|---|
| 10308.001.2-T | 34 |
| 10308.006.1-F | 35 |
| 1462.002.1-B | 230 |
| 2123.001.1-T | 316 |
| 22832.016.1-B | 341 |
| 2591.003.1-T | 454 |
| 3146.000.2-X | 593 |
| 3717.000.10-X | 664 |
| 3717.000.23-X | 666 |
| 51086.001.1-B | 811 |
| 51426.010.1-B | 855 |
| 54851.003.1-F | 951 |
| 597.000.1-Z | 1127 |
| 6093.031.1-B | 1136 |
| 6672.027.1-T | 1330 |
| 830.000.5-Z | 1498 |
| 9976.003.1-F | 1752 |

Another preferred example of all probes to detect Alzheimer's disease thus comprises:
from 1 to 3 probes, overlapping or not, each comprising all or part of sequence SEQ ID NO: 34 or of a sequence complementary thereto;
from 1 to 3 probes, overlapping or not, each comprising all or part of sequence SEQ ID NO: 35 or of a sequence complementary thereto;
from 1 to 3 probes, overlapping or not, each comprising all or part of sequence SEQ ID NO: 230 or of a sequence complementary thereto;
from 1 to 3 probes, overlapping or not, each comprising all or part of sequence SEQ ID NO: 316 or of a sequence complementary thereto;
from 1 to 3 probes, overlapping or not, each comprising all or part of sequence SEQ ID NO: 341 or of a sequence complementary thereto;
from 1 to 3 probes, overlapping or not, each comprising all or part of sequence SEQ ID NO: 454 or of a sequence complementary thereto;
from 1 to 3 probes, overlapping or not, each comprising all or part of sequence SEQ ID NO: 593 or of a sequence complementary thereto;
from 1 to 3 probes, overlapping or not, each comprising all or part of sequence SEQ ID NO: 664 or of a sequence complementary thereto;
from 1 to 3 probes, overlapping or not, each comprising all or part of sequence SEQ ID NO: 666 or of a sequence complementary thereto;
from 1 to 3 probes, overlapping or not, each comprising all or part of sequence SEQ ID NO: 811 or of a sequence complementary thereto;
from 1 to 3 probes, overlapping or not, each comprising all or part of sequence SEQ ID NO: 855 or of a sequence complementary thereto;
from 1 to 3 probes, overlapping or not, each comprising all or part of sequence SEQ ID NO: 951 or of a sequence complementary thereto;
from 1 to 3 probes, overlapping or not, each comprising all or part of sequence SEQ ID NO: 1127 or of a sequence complementary thereto;
from 1 to 3 probes, overlapping or not, each comprising all or part of sequence SEQ ID NO: 1136 or of a sequence complementary thereto;
from 1 to 3 probes, overlapping or not, each comprising all or part of sequence SEQ ID NO: 1330 or of a sequence complementary thereto;
from 1 to 3 probes, overlapping or not, each comprising all or part of sequence SEQ ID NO: 1498 or of a sequence complementary thereto; and
from 1 to 3 probes, overlapping or not, each comprising all or part of sequence SEQ ID NO: 1752 or of a sequence complementary thereto.

### Example 5

### Detection Protocol

The steps of the diagnostic test are as follows:

RNA is first extracted from blood samples collected in Paxgene tubes. These tubes contain a strong denaturing additive that stabilises the nucleic acids in vivo by reducing the breakdown of RNA in vitro and by minimising gene induction. When used with the Paxgene™ Blood RNA Kit, the samples taken enable exact detection and quantification of the level of gene transcription. After extraction, the quality of sample is assessed and the RNA is quantified, by procedures that are well known in the art.

The RNA is reverse transcribed, linearly amplified and then fragmented, labelled with biotin and finally hybridised on biochips containing the inventive probes. The chips are then washed, hybridisation is revealed and then they are scanned to measure the level of hybridisation on each probe. The hybridisation results are imported and analysed using Partek statistical analysis software.

Application of the signature gives a binary response that makes it possible to place the patient tested in the AD category or in the control category.

1-21. (canceled)

22. A method for detecting the presence of Alzheimer's disease in a mammal, comprising contacting, under conditions allowing hybridisation between complementary sequences, nucleic acids from a blood sample from the mammal and a set of probes to obtain an expression profile, the expression profile being characteristic of the presence of Alzheimer's disease in the mammal, the set of probes comprising:
at least one probe comprising all or part of SEQ ID NO: 34 or of the complement thereof;
at least one probe comprising all or part of SEQ ID NO: 230 or of the complement thereof;
at least one probe comprising all or part of SEQ ID NO: 341 or of the complement thereof;
at least one probe comprising all or part of SEQ ID NO: 454 or of the complement thereof;
at least one probe comprising all or part of SEQ ID NO: 664 or of the complement thereof;
at least one probe comprising all or part of SEQ ID NO: 811 or of the complement thereof;
at least one probe comprising all or part of SEQ ID NO: 951 or of the complement thereof;
at least one probe comprising all or part of SEQ ID NO: 1127 or of the complement thereof;
at least one probe comprising all or part of SEQ ID NO: 1136 or of the complement thereof; and
at least one probe comprising all or part of SEQ ID NO: 1752 or of the complement thereof,
wherein said part comprises at least 15 consecutive nucleotides of SEQ ID NO: 34, 230, 341, 454, 664, 811, 951, 1127, 1136 or 1752 or at least 15 consecutive nucleotides of the complement of SEQ ID NO: 34, 230, 341, 454, 664, 811, 951, 1127, 1136 or 1752.

23. The method according to claim 22, wherein the set of probes further comprises:
at least one probe comprising all or part of SEQ ID NO: 35 or of the complement thereof;
at least one probe comprising all or part of SEQ ID NO: 316 or of the complement thereof;
at least one probe comprising all or part of SEQ ID NO: 593 or of the complement thereof;
at least one probe comprising all or part of SEQ ID NO: 666 or of the complement thereof;
at least one probe comprising all or part of SEQ ID NO: 855 or of the complement thereof;
at least one probe comprising all or part of SEQ ID NO: 1330 or of the complement thereof; and
at least one probe comprising all or part of SEQ ID NO: 1498 or of the complement thereof,
wherein said part comprises at least 15 consecutive nucleotides of SEQ ID NO: 35, 316, 593, 666, 855, 1330 or 1498 or at least 15 consecutive nucleotides of the complement of SEQ ID NO: 35, 316, 593, 666, 855, 1330 or 1498.

24. The method according to claim 22, wherein the set of probes comprises at least one probe specific for each nucleic acid, or the complement thereof, of one of panels 1 to 14 defined in table 2, or of a distinctive fragment thereof having at least 15 consecutive nucleotides.

25. The method according to claim 22, wherein the set of probes comprises, for each target nucleic acid, a group of 1 to 3 specific probes that, optionally, overlap.

26. The method according to claim 22, wherein the probes are immobilised on support.

27. The method according to claim 22, wherein the expression profile is analysed by computer software methods.

28. The method according to claim 22, wherein the sample is a sample of whole blood.

29. A product comprising a support on which a set of probes is immobilised, the set comprising:
at least one probe comprising all or part of SEQ ID NO: 34 or of the complement thereof;
at least one probe comprising all or part of SEQ ID NO: 230 or of the complement thereof;
at least one probe comprising all or part of SEQ ID NO: 341 or of the complement thereof;
at least one probe comprising all or part of SEQ ID NO: 454 or of the complement thereof;
at least one probe comprising all or part of SEQ Ill NO: 664 or of the complement thereof;
at least one probe comprising all or part of SEQ ID NO: 811 or of the complement thereof;
at least one probe comprising all or part of SEQ ID NO: 951 or of the complement thereof;
at least one probe comprising all or part of SEQ ID NO: 1127 or of the complement thereof;
at least one probe comprising all or part of SEQ ID NO: 1136 or of the complement thereof, and
at least one probe comprising all or part of SEQ ID NC): 1752 or of the complement thereof,
wherein said part comprises at least 15 consecutive nucleotides of SEQ ID NO: 34, 230, 341, 454, 664, 811, 951, 1127, 1136 or 1752 or at least 15 consecutive nucleotides of the complement of SEQ ID NO: 34, 230, 341, 454, 664, 811, 951, 1127, 1136 or 1752.

30. A product comprising a support on which are immobilised at least two distinct nucleic acid probes, each probe comprising a sequence complementary to and/or specific to a target nucleic acid selected from SEQ ID NOS.: 1-1754 or the complement thereof.

31. The product according to claim 30, comprising a support on which are immobilised at least one set of distinct nucleic acid probes comprising a sequence complementary to and/or specific to the nucleic acids of any one of panels 1 to 14 defined in table 2.

32. The product according to claim 30, comprising a support on which are immobilised probes complementary to and/or specific for each of the 170 nucleic acids of PANEL 10 or the complement thereof.

33. The product according to claim 30, comprising a support on which are immobilised probes complementary to and/or specific for each of the 150 nucleic acids of PANEL 12 or the complement thereof.

34. The product according to claim 30, comprising a support on which are immobilised probes complementary to and/or specific for each of the 120 nucleic acids of PANEL 13 or the complement thereof.

35. The product according to claim 30, comprising a support on which immobilised probes complementary to and/or specific for each of the 250 nucleic acids of PANEL 14 or the complement thereof.

36. The product according to claim 30, wherein the probes are selected from SEQ. ID NOS.: 1755-6532.

37. A kit comprising a compartment or container comprising at least 5 distinct nucleic acids selected from SEQ ID NOS.: 1-6532.

38. A kit comprising a product according to claim 29 and reagents for a hybridisation reaction.

39. A method to detect the presence of Alzheimer's disease in a mammal, comprising contacting, under conditions allowing an amplification reaction, nucleic acids from a blood sample from said mammal and a set of primers, the set of primers comprising primers comprising all or part of each of the following target nucleic acid sequences, or of the complement thereof SEQ ID NOS.: 34, 230, 341, 454, 664, 811, 951, 1127, 1136 and 1752, to obtain an amplification profile or product, the amplification profile or product being characteristic of the presence of Alzheimer's disease in said mammal.
